(19) United States
(12) Patent Application Publication
(10) Pub. No.: US 2024/0307402 A1
(43) Pub. Date: Sep. 19, 2024

(54) PHARMACEUTICAL COMPOSITION FOR THE TREATMENT OF CANCER

(71) Applicant: IMPETIS BIOSCIENCES LTD., Mumbai (IN)

(72) Inventors: Kasim Mookhtiar, Mumbai (IN); Sujay Basu, Mumbai (IN); Sreekanth Rouduri, Mumbai (IN); Ashwin Meru, Mumbai (IN)

(21) Appl. No.: 18/431,910

(22) Filed: Feb. 2, 2024

Related U.S. Application Data
(63) Continuation of application No. 16/955,567, filed on Jun. 18, 2020, now abandoned.

(30) Foreign Application Priority Data
Dec. 19, 2017 (IN) 201721045725
Dec. 19, 2017 (IN) 201721045726
Dec. 19, 2017 (IN) 201721045727

(51) Int. Cl. A61K 31/522; A61K 39/395

(52) U.S. Cl. CPC A61K 31/522; A61K 39/3955

(57) ABSTRACT

The present disclosure discloses a pharmaceutical composition comprising compound of Formula I, Formula II, Formula III, Formula IV, and their pharmaceutically acceptable salts, analogs, tautomeric forms, stereoisomers, geometrical isomers, polymorphs, hydrates, solvates, metabolites, and prodrugs thereof, for the manufacture of a medicament for the treatment of a condition or disorder ameliorated by inhibition of the A2A/A2B receptor. It further discloses the use of the pharmaceutical composition of the present disclosure, for the preparation of a medicament for the treatment of a condition or disorder selected from prostate cancer, rectal cancer, renal cancer, ovarian cancer, endometrial cancer, thyroid cancer, pancreatic cancer, breast cancer, colon cancer, bladder cancer, brain cancer, glial cancer, melanoma cancer, pineal gland cancer, or lung cancer.

### FIELD OF THE INVENTION

The present disclosure relates to a pharmaceutical composition comprising compounds selected from the compound of Formula I, compound of Formula II, compound of Formula III, or Compound of Formula IV for the manufacture of a medicament for the treatment of a condition or disorder ameliorated by inhibition of the A2 A/A2 B receptor. In particular, it relates to the use of the pharmaceutical composition for the preparation of a medicament for the treatment of a condition or disorder selected from prostate cancer, rectal cancer, renal cancer, ovarian cancer, endometrial cancer, thyroid cancer, pancreatic cancer, breast cancer, colon cancer, bladder cancer, brain cancer, glial cancer, melanoma cancer, pineal gland cancer, or lung cancer.

### BACKGROUND OF INVENTION

Adenosine is an endogenous modulator of a wide range of physiological functions and is implicated in several pathologies. Recent advances in molecular biology coupled with several pharmacological studies have lead to identification of at least four subtypes of adenosine receptors, Ai, A2 A, A2 B and A3. The Ai and A3 receptors down-regulate cellular cAMP levels through their coupling to G protein, which inhibit adenylate cyclase. In contrast, A2 A and A2 B receptors couple to G protein that activate adenylate cyclase and increase intracellular levels of cAMP.

Advances in understanding the role of adenosine and its receptors in physiology and pathophysiology as well as new developments in medicinal chemistry of these receptors have identified potential therapeutic areas for drug development. With the combination of pharmacological data using selective ligands and genetically modified mice, important progress has been made towards understanding of the role of adenosine receptors (Ars) in a variety of diseases, such as inflammatory conditions, sepsis, heart attack, ischemia-reperfusion injury, vascular injury, spinal cord injury, chronic obstructive pulmonary disease (COPD), asthma, diabetes, obesity, inflammatory bowel disease, retinopathy, and Parkinson's Disease (PD).

In the central nervous system, A2 A antagonists can have antidepressant properties and stimulate cognitive functions. Epidemiological evidence shows a protective role for caffeine in Parkinson's disease. Moreover, A2 A receptor density is found to be very high in the basal ganglia which regulate motor control function. Hence, selective A2 A antagonists can improve motor impairment due to neurodegenerative diseases such as Parkinson's disease (Trends Pharmacol. Sci. 1997, 18, 338-344), senile dementia as in Alzheimer's disease, psychoses, stroke and be potentially effective in the treatment of cerebral ischacmia (Life Sci. 1994, 55, 61-65). A2a antagonists may also be employed for the treatment or management of attention related disorders such as attention deficit disorder and attention deficit hyperactivity disorder, extra pyramidal syndrome, e.g., dystonia, akathisia, pseudoparkinsonism and tardive dyskinesia, and disorders of abnormal movement such as restless leg syndrome and periodic limb movement in sleep. Several of these indications have been disclosed in patent applications (eg. WO 02/055083, WO 05/044245 and WO 06/132275). Adenosine A2 A antagonists could also be useful in the treatment of amyotrophic lateral sclerosis, cirrhosis, and fibrosis and fatty liver (US 2007037033, WO 01/058241). A2 A receptor antagonists are also useful for the mitigation of addictive behavior (WO 06/009698) and for the treatment and prevention of dermal fibrosis in diseases such as scleroderma (Arthritis & Rheumatism, 54(8), 2632-2642, 2006).

Parkinson's disease (PD) is a progressive, incurable disorder with no definite preventive treatment, although drugs are available to alleviate the symptoms and/or slow down the progress of the disease. Among the various strategies, A2 A AR blockers are considered a potential approach to treatment of the disease. Within the brain A2 A ARS are richly expressed in the striatum, nucleus accumbens, and olfactory tubercle. Co-expression of A2 A with D2 dopamine receptors has been reported in the GABAergic striatopallidal neurons where adenosine and dopamine agonists exert antagonistic effects in the regulation of locomotor activity. Activation of A2 A ARS in striatopallidal neurons decreases the affinity of D2 receptors for dopamine, antagonizing the effects of D2 receptors. The negative interaction between A2 A and D2 receptors is at the basis of the use of A2 A antagonists as a novel therapeutic approach in the treatment of PD (Pharmacol. Ther. 2005, 105, 267). The recent discovery that the A2 A can form functional heteromeric receptor complexes with other Gprotein-coupled receptors such as D2 receptors and the mGlu5 receptors has also suggested new opportunities for the potential of A2 A antagonists in PD (J. Mol. Neurosci. 2005, 26, 209).

Adenosine signaling is known to serve apoptotic, angiogenic and pro-inflammatory functions and might be relevant to the pathogenesis of asthma and chronic obstructive pulmonary disease (Trends in Pharmacological Sciences, 2003, 24, 8). Extracellular adenosine acts as a local modulator with a generally cytoprotective function in the body. Its effects on tissue protection and repair fall into four categories: increasing the ratio of oxygen supply to demand; protecting against ischaemic damage by cell conditioning; triggering anti-inflammatory responses; and the promotion of angiogenesis.

In recent years, A2 A receptor has shown exciting progress in the development of immunotherapy for the treatment of cancer (Cancer Immunol Res. 2015, 3, 506-517). Adenosine generation in tumor microenvironment is an active metabolic mechanism used by cancer cells to avoid anti-tumor immuno surveillance and increase metastasis. Ectonucleotidase CD73 and CD39 (highly expressed on tumor cells and stromal cells) convert ATP released by dying tumor cells to adenosine. Adenosine signaling through A2 A receptors enhances pro-tumoral responses in the tumor microenvironment contributing to tumor growth and metastases. A2 A receptors are expressed on several immune cell types-T lymphocytes, dendritic cells, natural killer cells. A2 A receptor activation on T cells and NK cells causes immunosuppression by reducing their proliferation, cytokine production and tumor killing activity. A2 A antagonists could induce anti-tumoral responses in multiple types of cancer when used as stand alone or in combination with existing immunotherapies or radiotherapy or chemotherapy. Cancers that could benefit from A2 A antagonist therapy include melanoma, triple negative breast cancer, colon cancer, colorectal cancer, lung cancer, prostate cancer, renal cell cancer, non-small cell lung cancer, bladder cancer, cervical, vulvar or anal cancer, esophageal cancer, metastatic head and neck cancer, liver cancer, lymphoma, multiple myeloma, ovarian cancer, pancreatic cancer, acute myeloid leukemia, Kaposi sarcoma.

The A2 B adenosine receptor subtype (Feoktistov, et al. I. Pharmacol. Rev. 1997, 49, 381-402) has been identified in a variety of human and murine tissues and is involved in the regulation of vascular tone, smooth muscle growth, angiogenesis, hepatic glucose production, bowel movement, intestinal secretion, and mast cell degranulation.

A2B receptors have been implicated in mast cell activation and asthma, control of vascular tone, cardiac myocyte contractility, cell growth and gene expression, vasodilation, regulation of cell growth, intestinal function, and modulation of neuro secretion (Pharmacological Reviews, 2003, 49, 4).

A2 B receptors modulate mast cell function. Adenosine activates adenylate cyclase and protein kinase C and potentiates stimulated mediator release in mouse bone marrow derived mast cells. Activation of A2 B receptors in HMC-1 augments IL-8 release and potentiates PMA-induced secretion of IL-8. Thus, adenosine would contribute to the asthmatic response by acting on the mast cell to enhance the release of proinflammatory mediators. (Pulmonary Pharmacology & Therapeutics 1999, 12, 111-114). In COPD, transformation of pulmonary fibroblasts into myofibroblasts is considered a major mechanism. Activation of the A2 B AR is involved in this process. Selective A2 B antagonists are expected to have beneficial effect on pulmonary fibrosis (Curr . Drug Targets, 2006, 7, 699-706; Am. J. Resper. Cell. Mol. Biol., 2005, 32, 228). A2 B antagonists can be used as wound healing agents. Activation of the A2 B AR promotes angiogenesis by increasing the release of angiogenic factors and A2 B antagonists are useful to block angiogenesis (Circ . Res., 2002, 90, 531-538). A2 B AR may be involved in the inhibition cardiac fibroblast (CF) proliferation (Am.J . Physiol. Heart Circ. Physiol., 2004, 287, H2478-H2486). Adenosine stimulates Cl-secretion in the intestinal epithelia pointing towards a possible treatment for cystic fibrosis patients with CFTR mutation (Am. J. Respir. Cell Mol. Biol., 2008, 39, 190-197). High affinity A2 B antagonists are effective in hot plate model suggestive of the role of A2 B in nociception and can be used as potential analgesic agents (The J. of Pharmacol and Exp. Ther., 2004, 308, 358-366).

A2B receptor is involved in release of IL-6. Increasing evidence suggests that IL-6 plays a role in Alzheimer's disease in the context of inflammatory process associated with disease. Hence A2B receptor antagonist might be useful for Alzheimer's disease.

The A2B ARS are involved in the stimulation of nitric oxide production during Na+-linked glucose or glutamine absorption. They are involved in glucose production in hepatocytes upon agonist stimulation. A2B-receptor antagonists showed an anti-diabetic potential mainly by increasing plasma insulin levels under conditions when the adenosine tonus was elevated in-vivo and increased insulin release in-vitro (J Pharm. Pharmacol. 2006 December; 58(12): 1639-45). Thus, A2B antagonists may serve as a novel target for the treatment of this metabolic disease.

It has been demonstrated that adenosine activation of the A2B adenosine receptor increase cAMP accumulation, cell proliferation and VEGF expression in human retinal endothelial cells. Activation of A2B AdoR increased vascular endothelial cell growth factor mRNA and protein expression in human retinal endothelial cells. Adenosine also has a synergistic effect with VEGF on retinal endothelial cell proliferation and capillary morphogenesis in vitro. Such activity is necessary in healing wounds, but the hyperproliferation of endothelial cells promotes diabetic retinopathy. Also, an undesirable increase in blood vessels occurs in neoplasia. Accordingly, inhibition of binding of adenosine to A2B receptors in the endothelium will alleviate or prevent hypervasculation, thus preventing retinopathy and inhibiting tumor formation.

Adenosine generation in tumor microenvironment is an active metabolic mechanism used by cancer cells to avoid anti-tumor immunosurveillance and increase metastasis. Ecto nucleotidase CD73 and CD39 (highly expressed on tumor cells and stromal cells) convert ATP released by dying tumor cells to adenosine. A2B receptors are expressed at low levels on multiple cell types under normal conditions, but significantly upregulated under hypoxic conditions that prevail in the tumor microenvironment. Activation of A2B receptors promotes angiogenesis and causes T cell and myeloid derived suppressor cell (MDSC) mediated immunosuppression in the tumor microenvironment. A2B antagonists could induce anti-tumoral responses in multiple types of cancer when used as stand alone or in combination with existing immunotherapies or radiotherapy or chemotherapy. Cancers that could benefit from A2B antagonist therapy include melanoma, triple negative breast cancer, colon cancer, colorectal cancer, lung cancer, prostate cancer, renal cell cancer, non-small cell lung cancer, bladder cancer, cervical, vulvar or anal cancer, esophageal cancer, metastatic head and neck cancer, liver cancer, lymphoma, multiple myeloma, ovarian cancer, pancreatic cancer, acute myeloid leukemia, Kaposi sarcoma.

Adenosine A2B receptors are ubiquitous and regulate multiple biological activities. For example, adenosine binds to A2B receptors on endothelial cells, thereby stimulating angiogenesis. Adenosine also regulates the growth of smooth muscle cell populations in blood vessels. Adenosine stimulates A2B receptors on mast cells, thus modulating Type I hypersensitivity reactions. Adenosine also stimulates gastrosecretory activity by ligation with A2B in the intestine.

While many of these biological effects of adenosine are necessary to maintain normal tissue homeostasis, under certain physiological changes it is desirable to modulate its effects. For example, the binding of A2B receptors stimulates angiogenesis by promoting the growth of endothelial cells. Such activity is necessary in healing wounds, but the hyperproliferation of endothelial cells promotes diabetic retinopathy. Also, an undesirable increase in blood vessels occurs in neoplasia. Accordingly, inhibition of the binding of adenosine to A2B receptors in the endothelium will alleviate or prevent hypervasculation, thus preventing retinopathy and inhibibiting tumor formation.

A2B receptors are found in the colon in the basolateral domains of intestinal epithelial cells, and when acted upon by the appropriate ligand act to increase chloride secretion, thus causing diarrhea, which is a common and potentially fatal complication of infectious diseases such as cholera and typhus. A2B antagonists can therefore be used to block intestinal chloride secretion and are thus useful in the treatment of inflammatory gastrointestinal tract disorders, including diarrhea. Another adverse biological effect of adenosine acting at the A2B receptor is the over-stimulation of cerebral IL-6, a cytokine associated with dementias and Alzheimer's disease.

Accordingly, it is desired to provide compounds that are potent A2 A/A2B antagonists (i.e., compounds that inhibit the A2 A/A2B adenosine receptor), fully or partially selective for the A2 A/A2B receptor, useful in the treatment of various disease states related to modulation of the A2 A/A2B receptor, for example cancer.

### SUMMARY OF THE INVENTION

In an aspect of the present disclosure, there is provided a pharmaceutical composition comprising compound of Formula I and its pharmaceutically acceptable salt, analog, tautomeric form, stereoisomer, geometrical isomer, polymorph, hydrate, solvate, metabolite, and prodrug thereof

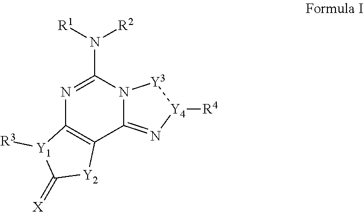

wherein
represents a single bond or a double bond;
X is selected from O, S or NRa; Yi is selected from N or CH; Y2 is selected from NR5, O or CR5 R6;
Y3 is selected from N, CH, CH2, C(=0), or C(═S); Y4 is selected from N, C, or CH; R1 and R2 are independently selected from hydrogen or alkyl; R3 is -A-Z-B-Q; wherein, A is absent or is a group selected from alkylene, alkenylene, or alkynylene; wherein one or more methylene groups is optionally replaced by hetero atoms or groups such as -0-, —S(0)p-, —N(Ra)—, or —C(O); alkylene, alkenylene and alkynylene is optionally substituted with —(CRd Re)n OR7, (CRd Re)n COOR7, —(CRd Re)n NR8 R9, cyano, halogen, haloalkyl, perhaloalkyl, alkoxyalkoxy, alkyl, or cycloalkyl; Z is absent or is selected from a cycloalkyl or a heterocyclyl; wherein cycloalkyl and heterocyclyl are unsubstituted or substituted independently with 1, 2, or 3 substituents independently selected from alkyl, alkenyl, alkynyl, acyl, —(CRd Re)n OR7, (CRd Re)n COOR7, —(CRd Re)n NR8 R9, aminocarbonyl, alkoxycarbonylamino, halogen, haloalkyl, perhaloalkyl, azido, cyano, keto, thiocarbonyl, —S03 H, aminocarbonylamino, nitro, —S(0)2 NRa Ra, —NRb S(0)2 Rb or —S(0)P Rc;
B is absent or is a group selected from alkylene, alkenylene or alkynylene; wherein one or more methylene groups is optionally replaced by hetero atoms or groups such as -0-, —S(0)p-, —N(Ra)—, or —C(O); alkylene, alkenylene and alkynylene is optionally substituted with hydroxy, amino, aminoalkyl, cyano, halogen, haloalkyl, perhaloalkyl, carboxy, carboxyalkyl, alkoxy, hydroxyalkyl, alkoxyalkyl, alkoxyalkoxy or alkyl;
Q is selected from hydrogen, alkyl, cycloalkyl, cycloalkylalkyl, aryl, arylalkyl, heterocyclyl, heterocyclylalkyl, heteroaryl or heteroarylalkyl; wherein alkyl, cycloalkyl, cycloalkylalkyl, aryl, arylalkyl, heterocyclyl, heterocyclylalkyl, heteroaryl and heteroarylalkyl are unsubstituted or substituted independently with 1, 2, or 3 substituents independently selected from alkyl, alkenyl, alkynyl, halogen, haloalkyl, perhaloalkyl, azido, cyano, nitro, keto, thiocarbonyl, cyanoalkyl, cyanoalkylcarbonyl, —(CRd Re)n OR7, —(CRd Re)n C(0)R7, —(CRd Re)n SR7, —(CRd Re)n COOR7, —(CRd Re)n NR8 R9, —(CRd Re)n C(0)NR8 R9, —(CRd Re)n NR8 C(0)OR7, —(CRd Re), NR8 C(0)NR8 R9, —NRb S(0)2 Rb, —S(0)P Rc, —SO3H, —S(0)2 NRa Ra, cycloalkyl, cycloalkenyl, cycloalkylalkyl aryl, arylalkyl, heterocyclyl, heterocyclylalkyl, heteroaryl or heteroarylalkyl; wherein cach substituent is unsubstituted or substituted with 1, 2, or 3 substituents independently selected from alkyl, carboxy, carboxyalkyl, aminocarbonyl, hydroxy, alkoxy, halogen, haloalkyl, perhaloalkyl, haloalkoxy, perhaloalkoxy, amino, substituted amino, cyano or —S(0)P Rc;
R4 is selected from the group consisting of hydrogen, alkyl, alkenyl, alkynyl, haloalkyl, hydroxyalkyl, carboxyalkyl, cycloalkyl, cycloalkylalkyl, aryl, arylalkyl, heterocyclyl, heterocyclylalkyl, heteroaryl and heteroarylalkyl; wherein alkyl, alkenyl, alkynyl, cycloalkyl, cycloalkylalkyl, arylalkyl, aryl, heteroaryl, heteroarylalkyl, heterocyclyl, and heterocyclylalkyl are unsubstituted or substituted independently with up to four substituents independently selected from alkyl, alkenyl, alkynyl, acyl, —(CRd Re)n OR7, (CRd Re)n COOR7, —(CRd Re)n NR8 R9, aminocarbonyl, alkoxycarbonylamino, aminocarbonylamino, azido, cyano, halogen, haloalkyl, perhaloalkyl, keto, nitro, —S(0)2 NRb Rb, —NRb S(0)2 Rb or —S(0)p Rc, thiocarbonyl, —S03 H, cycloalkyl, cycloalkenyl, aryl, heteroaryl or heterocyclyl;
R5 and R6 are independently selected from the group consisting of hydrogen, hydroxy, —(CRd Re) n OR7, (CRd Re)n COOR7, —(CRd Re)n NR8 R9, cyanoalkyl, haloalkyl, alkoxyalkoxyalkyl, alkyl, alkenyl, alkynyl, cycloalkyl, cycloalkylalkyl, aryl, arylalkyl, heterocyclyl, heterocyclylalkyl, heteroaryl and heteroarylalkyl;
R7 is selected from hydrogen, alkyl, halogen, haloalkyl, —(CRd Re)n OR7, —(CRd Re)n COOR7, —(CRe Re)n C(0)R7, carbonylamino, cycloalkyl, cycloalkylalkyl, aryl, arylalkyl, heteroaryl, heteroarylalkyl, heterocyclyl or heterocyclylalkyl;
R8 and R9 are independently selected from the group consisting of hydrogen, alkyl, haloalkyl, —(CRd Rc)n OR7, —(CRd Re)n C(0)R7, aryl, arylalkyl, heteroaryl, heteroarylalkyl, cycloalkyl, cycloalkylalkyl, heterocyclyl and heterocyclylalkyl, or R8 and R9 taken together form a monocyclic or a bicyclic ring system which is saturated or partially unsaturated and optionally have additional heteroatoms selected from O, N or S, said ring system is further optionally substituted with 1 to 4 substituents independently selected from halo, alkyl, alkenyl, alkynyl, nitro, cyano, —(CRd Re)n OR7, —(CRd Re)n SR7, —(CRd Re)n NR8 R9, oxo, alkylsulfonyl, —(CRd Re)n COOR7, —(CRd Re)n C(0)NR8 R9, cycloalkyl, cycloalkylalkyl, aryl, arylalkyl, heterocyclyl, heterocyclylalkyl, heteroaryl, or heteroarylalkyl;
Ra is selected from hydrogen or alkyl; Rb each is independently selected from the group consisting of hydrogen, alkyl, acyl, carboxyalkyl, carbonylamino, cycloalkyl, cycloalkylalkyl, aryl, arylalkyl, heteroaryl, heteroarylalkyl, heterocyclyl and heterocyclylalkyl; Rc is selected from alkyl, cycloalkyl, aryl, heterocyclyl or heteroaryl; Rd and Re are independently selected from the group consisting of hydrogen, —OR7, halogen, haloalkyl, perhaloalkyl and alkyl; n is 0, 1, 2, 3 or 4, and
p is 0, 1 or 2,
for the manufacture of a medicament for the treatment of a condition or disorder ameliorated by inhibition of the A2A/A2B receptor.

In an aspect of the present disclosure, there is provided a pharmaceutical composition comprising compound of Formula II and its pharmaceutically acceptable salts, analogs, tautomeric forms, stereoisomers, geometrical isomers, polymorphs, hydrates, solvates, metabolites, and prodrugs thereof

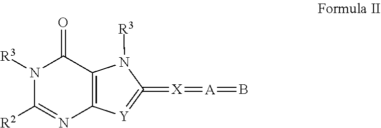

wherein,
Y is selected from N or CR; R is selected from H, hydroxy, alkoxy, alkyl, or aryl; R1 is selected from a group consisting of alkyl, alkenyl and alkynyl, wherein one or more methylene groups are optionally replaced by hetero atoms or groupselected from -0-, —S(0)p-, —N(Ra)—, or —C(O) provided that the heteroatom is not adjacent to N in the ring; p is selected from 0, 1 or 2; wherein alkyl, alkenyl and alkynyl are unsubstituted or substituted independently with alkoxy, acyl, acylamino, acyloxy, amino, monoalkylamino, dialkylamino, aminocarbonyl, alkoxycarbonylamino, azido, cyano, halogen, haloalkyl, hydroxy, hydroxyalkyl, keto, thiocarbonyl, carboxy, alkylcarboxy, carboxyalkyl, —S03 H, aminocarbonylamino, hydroxyamino, alkoxyamino, nitro, —S(0)2 NRa Ra, —NRa S(0)2 Ra, or —S(0)p Ra;
R2 is selected from a group consisting of hydrogen, halogen, cyano, nitro, carboxy, acyl, aminocarbonyl, alkyl, alkenyl, alkynyl, hydroxyalkyl, carboxyalkyl, haloalkyl, haloalkyloxy, alkoxy, —NRb Rb, —S(0)p Rb, cycloalkyl, cycloalkylalkyl, cycloalkyloxy, aryl, arylalkyl, aryloxy, heterocyclyl, heterocyclylalkyl, heterocyclyloxy, heteroaryl, heteroarylalkyl and heteroaryloxy; wherein alkyl, alkenyl, alkynyl, alkoxy, carboxyalkyl, cycloalkyl, cycloalkylalkyl, cycloalkyloxy, aryl, arylalkyl, aryloxy, heterocyclyl, heterocyclylalkyl, heterocyclyloxy, heteroaryl, heteroarylalkyl, heteroaryloxy and Rb are unsubstituted or substituted independently with alkyl, alkenyl, alkynyl, alkoxy, acyl, acylamino, acyloxy, nitro, amino, monoalkylamino, dialkylamino, hydroxyamino, alkoxyamino, aminocarbonylamino, azido, cyano, halogen, hydroxy, hydroxyalkyl, keto, thiocarbonyl, carboxy, alkylcarboxy, carboxyalkyl, —S03 H, arylamino, cycloalkylamino, heteroarylamino, heterocyclylamino, aminocarbonyl, alkoxycarbonylamino, cycloalkyl, cycloalkyloxy, cycloalkenyl, aryl, aryloxy, heteroaryl, heteroaryloxy, heterocyclyl, heterocyclyloxy, —S(0)2 NRc Rc, —NRc S(0)2 Rc or —S(0)p Rd; wherein cach substituent is unsubstituted or substituted with 1, 2, or 3 substituents independently selected from alkyl, carboxy, carboxyalkyl, aminocarbonyl, hydroxy, alkoxy, halogen, haloalkyl, haloalkoxy, amino, substituted amino, cyano or —S(0)p Rd;
R3 is selected from a group consisting of hydrogen, alkyl, alkenyl, alkynyl, alkoxyalkyl, cycloalkyl, cycloalkylalkyl, aryl, arylalkyl, heteroaryl and heteroarylalkyl; wherein alkyl, alkenyl, alkynyl, alkoxyalkyl, cycloalkyl, cycloalkylalkyl, aryl, arylalkyl, heteroaryl, and heteroarylalkyl are unsubstituted or substituted independently with alkyl, alkenyl, alkynyl, alkoxy, cycloalkyl, cycloalkenyl, acyl, acylamino, acyloxy, amino, monoalkylamino, dialkylamino, arylamino, cycloalkylamino, heteroarylamino, heterocyclylamino, aminocarbonyl, alkoxycarbonylamino, azido, cyano, halogen, hydroxy, hydroxyalkyl, keto, thiocarbonyl, carboxy, alkylcarboxy, carboxyalkyl, carboxyalkyloxy, alkylcarboxyalkyloxy, —SO3H, aryl, aryloxy, cycloalkyloxy, heteroaryl, aminocarbonylamino, heteroaryloxy, heterocyclyl, heterocyclyloxy, hydroxyamino, alkoxyamino, nitro, S(0)2 NRc Rc, —NRc S(0)2 Rc or —S(0)p Rd; wherein cach substituent is unsubstituted or substituted with 1, 2, or 3 substituents independently selected from alkyl, carboxy, carboxyalkyl, aminocarbonyl, hydroxyd, alkoxy, halogen, haloalkyl, haloalkoxy, amino, substituted amino, cyano or —S(0)p Rd;
X is either an optionally substituted arylene or an optionally substituted heteroarylene;
A is selected from a bond, (Ci-C6)alkylene, (C2-C6)alkenylene or (C2-Cejalkynylenc group, wherein 1 to 4 methylene groups are optionally replaced by groups independently selected from O, —S(0)p-, —N(Rb)—, or —C(O)—; wherein alkylene, alkenylene, and alkynylene are unsubstituted or substituted independently with alkyl, alkenyl, alkynyl, alkoxy, cycloalkyl, cycloalkenyl, acyl, acylamino, acyloxy, amino, monoalkylamino, dialkylamino, arylamino, cycloalkylamino, heteroarylamino, heterocyclylamino, aminocarbonyl, alkoxycarbonylamino, azido, cyano, halogen, hydroxy, hydroxyalkyl, kcto, thiocarbonyl, carboxy, alkylcarboxy, carboxyalkyl, carboxyalkyloxy, alkylcarboxyalkyloxy —S03 H, aryl, aryloxy, cycloalkyloxy, heteroaryl, aminocarbonylamino, heteroaryloxy, heterocyclyl, heterocyclyloxy, hydroxyamino, alkoxyamino, nitro, S(0)2 NRc Rc, —NRc S(0)2 Rc or —S(0)p Rd; wherein each substituent is unsubstituted or substituted with 1, 2, or 3 substituents independently selected from alkyl, carboxy, carboxyalkyl, aminocarbonyl, hydroxy, alkoxy, halogen, CF3, amino, substituted amino, cyano or —S(0)p Rd;
B is selected from hydrogen, heterocyclyl, cycloalkyl, aryl or heteroaryl; wherein heterocyclyl, cycloalkyl, aryl and heteroaryl are unsubstituted or substituted independently with alkyl, alkenyl, alkynyl, alkoxy, cycloalkyl, cycloalkylalkyl, cycloalkenyl, acyl, acylamino, acyloxy, amino, monoalkylamino, dialkylamino, arylamino, cycloalkylamino, heteroarylamino, heterocyclylamino, aminocarbonyl, alkoxycarbonylamino, azido, cyano, halogen, hydroxy, hydroxyalkyl, keto, thiocarbonyl, carboxy, alkylcarboxy, carboxyalkyl, carboxyalkyloxy, alkylcarboxyalkyloxy —SO3H, aryl, arylalkyl, aryloxy, cycloalkyloxy, heteroaryl, heteroarylalkyl, aminocarbonylamino, heteroaryloxy, heterocyclyl, heterocyclylalkyl, heterocyclyloxy, hydroxyamino, alkoxyamino, nitro, S(0)2 NRb Rb, —NRb S(0)2 Rb or —S(0)p Rd; wherein each substituent is unsubstituted or substituted with 1, 2, or 3 substituents independently selected from alkyl, carboxy, carboxyalkyl, aminocarbonyl, hydroxy, alkoxy, alkoxyalkoxy, alkoxyalkyl, halogen, haloalkyl, haloalkoxy, amino, substituted amino, cyano or —S(0)P Rd;
Ra is independently selected from hydrogen or alkyl; Rb is independently selected from the group consisting of hydrogen, alkyl, acyl, carboxyalkyl, carbonylamino, cycloalkyl, cycloalkylalkyl, aryl, arylalkyl, heteroaryl, heteroarylalkyl, heterocyclyl, and heterocyclylalkyl;
Rc is selected from hydrogen, alkyl, aryl, heteroaryl or heterocyclyl;
Rd is selected from alkyl, cycloalkyl, aryl, heterocyclyl or heteroaryl; and p is 0, 1 or 2, for the manufacture of a medicament for the treatment of a condition or disorder ameliorated by inhibition of the A2A/A2B receptor.

In an aspect of the present disclosure there is provided a pharmaceutical composition comprising compound of Formula III or IV and its pharmaceutically acceptable salts, analogs, tautomeric forms, stereoisomers, geometrical isomers, polymorphs, hydrates, solvates, metabolites, and prodrugs thereof

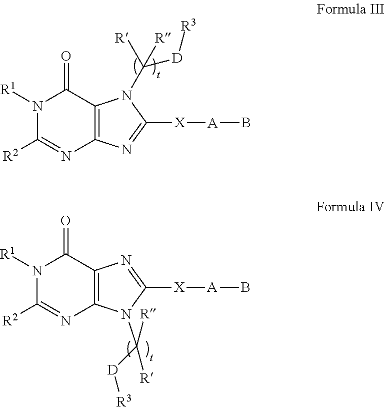

wherein,
R1 is an alkyl wherein one or more methylene groups are optionally replaced by hetero atoms or group selected from -0-, —S(0)p-, —N(Ra)—, or —C(O), provided that the heteroatom is not adjacent to N in the ring; p is selected from 0, 1 or 2;
wherein alkyl is unsubstituted or substituted with alkoxy, acyl, acylamino, acyloxy, amino, monoalkylamino, dialkylamino, aminocarbonyl, alkoxycarbonylamino, azido, cyano, halogen, haloalkyl, hydroxy, hydroxyalkyl, keto, thiocarbonyl, carboxy, alkylcarboxy, carboxyalkyl, -aminocarbonylamino, hydroxyamino, alkoxy amino;
R2 is selected from the group consisting of hydrogen, halogen, cyano, nitro, carboxy, acyl, aminocarbonyl, alkyl, alkenyl, alkynyl, hydroxyalkyl, carboxyalkyl, haloalkyl, haloalkyloxy, alkoxy, —NRb Rb, —S(0)p Rb, cycloalkyl, cycloalkylalkyl, cycloalkyloxy, aryl, arylalkyl, aryloxy, heterocyclyl, heterocyclylalkyl, heterocyclyloxy, heteroaryl, heteroarylalkyl and heteroaryloxy; wherein alkyl, alkenyl, alkynyl, alkoxy, carboxyalkyl, cycloalkyl, cycloalkylalkyl, cycloalkyloxy, aryl, arylalkyl, aryloxy, heterocyclyl, heterocyclylalkyl, heterocyclyloxy, heteroaryl, heteroarylalkyl, heteroaryloxy and Rb are unsubstituted or substituted independently with alkyl, alkenyl, alkynyl, alkoxy, acyl, acylamino, acyloxy, nitro, amino, monoalkylamino, dialkylamino, hydroxyamino, alkoxyamino, aminocarbonylamino, azido, cyano, halogen, hydroxy, hydroxyalkyl, keto, thiocarbonyl, carboxy, alkylcarboxy, carboxyalkyl, —S03 H, arylamino, cycloalkylamino, heteroarylamino, heterocyclylamino, aminocarbonyl, alkoxycarbonylamino, cycloalkyl, cycloalkyloxy, cycloalkenyl, aryl, aryloxy, heteroaryl, heteroaryloxy, heterocyclyl, heterocyclyloxy, —S(0)2 NRc Rc, —NRc S(0)2 Rc or —S(0)p Rd; wherein cach substituent is unsubstituted or substituted with 1, 2, or 3 substituents independently selected from alkyl, carboxy, carboxyalkyl, aminocarbonyl, hydroxy, alkoxy, halogen, haloalkyl, haloalkoxy, amino, substituted amino, cyano or —S(0)p Rd;
R′ and R″ are independently selected from hydrogen, or alkyl; or
R′ and R″ taken together may represent O, or a lower cycloalkyl ring system which is saturated or partially unsaturated;
R3 is selected from the group consisting of alkyl, aryl, —C(0)R4 and -P(0)(OR5)2;
R4 is selected from alkyl, alkoxy, aryl, heteroaryl, heterocyclyl, or -NR6 R7;
R5 is selected from hydrogen, alkyl, aryl, arylalkyl, —CH2 OC(0)alkyl, or —CH2 0C(0)Oalkyl; or two R5 groups taken together form a five or six membered ring system which is saturated or partially unsaturated and is optionally substituted with 1 to 4 substituents independently selected from halo, alkyl, aryl or heteroaryl; R6 and R7 are independently selected from the group consisting of hydrogen, alkyl, heterocyclyl and heterocyclylalkyl; or
R6 and R7 taken together form a monocyclic ring system which is saturated or partially unsaturated and optionally have additional heteroatoms selected from O, Nor S, wherein the ring system is optionally substituted with 1 to 4 substituents independently selected from halo, alkyl, alkoxy, or -NR8 R9; R4, R5, R6 and R7 is optionally substituted with 1 to 4 substituents independently selected from hydroxyl, halogen, alkyl, alkoxy, haloalkyl, -NR8 R9, —C(0)OR10, —OC(0)R10 or —NC(0)R10;
R8 and R9 are independently selected from the group consisting of hydrogen and alkyl;
R10 is selected from hydrogen, hydroxy, halogen, amino, substituted amino, cyano, alkyl, alkoxy, haloalkyl, haloalkoxy, carboxy, carboxyalkyl, aminocarbonyl, aryl or arylalkyl;
X is an optionally substituted arylene or an optionally substituted heteroarylene;
A is selected from a bond, or (Ci-C6)alkylene, wherein 1 to 4 methylene groups are optionally replaced by group independently selected from O, —S(0)p-, —N(Rb)—, or —C(O)—; wherein alkylene is unsubstituted or substituted independently with alkyl, alkonyl, alkynyl, alkoxy, cycloalkyl, cycloalkenyl, acyl, acylamino, acyloxy, amino, monoalkylamino, dialkylamino, arylamino, cycloalkylamino, heteroarylamino, heterocyclylamino, aminocarbonyl, alkoxycarbonylamino, azido, cyano, halogen, hydroxy, hydroxyalkyl, keto, thiocarbonyl, carboxy, alkylcarboxy, carboxyalkyl, carboxyalkyloxy, alkylcarboxyalkyloxy —S03 H, aryl, aryloxy, cycloalkyloxy, heteroaryl, aminocarbonylamino, heteroaryloxy, heterocyclyl, heterocyclyloxy, hydroxyamino, alkoxyamino, nitro, S(0)2 NRc Rc, —NRc S(0)2 Rc or —S(0)p Rd;
wherein cach substituent is unsubstituted or substituted with 1, 2, or 3 substituents independently selected from alkyl, carboxy, carboxyalkyl, aminocarbonyl, hydroxy, alkoxy, halogen, CF3, amino, substituted amino, cyano or —S(0)p Rd;
B is selected from hydrogen, heterocyclyl, cycloalkyl, aryl or heteroaryl; wherein heterocyclyl, cycloalkyl, aryl and heteroaryl are unsubstituted or substituted independently with alkyl, alkonyl, alkynyl, alkoxy, cycloalkyl, cycloalkylalkyl, cycloalkenyl, acyl, acylamino, acyloxy, amino, monoalkylamino, dialkylamino, arylamino, cycloalkylamino, heteroarylamino, heterocyclylamino, aminocarbonyl, alkoxycarbonylamino, azido, cyano, halogen, hydroxy, hydroxyalkyl, keto, thiocarbonyl, carboxy, alkylcarboxy, carboxyalkyl, carboxyalkyloxy, alkylcarboxyalkyloxy—SO3H, aryl, arylalkyl, aryloxy, cycloalkyloxy, heteroaryl, heteroarylalkyl, aminocarbonylamino, heteroaryloxy, heterocyclyl, heterocyclylalkyl, heterocyclyloxy, hydroxyamino, alkoxyamino, nitro, S(0)2 NRb Rb, —NRb S(0)2 Rb or —S(0)p Rd; wherein cach substituent is unsubstituted or substituted with 1, 2, or 3 substituents independently selected from alkyl, carboxy, carboxyalkyl, aminocarbonyl, hydroxy, alkoxy, alkoxyalkoxy, alkoxyalkyl, halogen, haloalkyl, haloalkoxy, amino, substituted amino, cyano or —S(0)P Rd;
D is selected from -0-, —S(0)p-, or —N(Ra)—;
Ra is hydrogen or an alkyl;
Rb is selected from the group consisting of hydrogen, alkyl, acyl, carboxyalkyl, carbonylamino, cycloalkyl, cycloalkylalkyl, aryl, arylalkyl, heteroaryl, heteroarylalkyl, heterocyclyl and heterocyclylalkyl;
Rc is selected from hydrogen, alkyl, aryl, heteroaryl or heterocyclyl;
Rd is selected from alkyl, cycloalkyl, aryl, heterocyclyl or heteroaryl;
p is 0, 1 or 2; and
t is 1 or 2, for the manufacture of a medicament for the treatment of a condition or disorder ameliorated by inhibition of the A2A/A2B receptor.

In an aspect of the present disclosure there is provided a method of using the pharmaceutical composition of the present disclosure comprising a compound selected from compound of Formula I, Formula II, Formula III, or Formula IV and their pharmaceutically acceptable salt, analog, tautomeric form, stercoisomer, geometrical isomer, polymorph, hydrate, solvate, metabolite, and prodrug thereof, in the treatment of a disease or condition in a mammal that is amenable to treatment with an A2A/A2B receptor antagonist, the method comprising: administering to a mammal in need thereof a therapeutically effective dose of the pharmaceutical composition of the present dislcosure.

In an aspect of the present disclosure there is provided a method of treatment of a disorder or condition ameliorated by antagonizing the A2A/A2B receptor, the method comprising: administering an effective amount of the pharmaceutical composition of the present disclosure comprising a compound selected from compound of Formula I, Formula II, Formula III, or Formula IV and their pharmaceutically acceptable salts, analog, tautomeric form, stereoisomer, geometrical isomer, polymorph, hydrate, solvate, metabolite, and prodrug thereof, to a patient in need of such treatment.

In an aspect of the present disclosure there is provided use of the pharmaceutical composition of the present disclosure comprising compound selected from compound of Formula I, Formula II, Formula III, or Formula IV and their pharmaceutically acceptable salt, analog, tautomeric form, stereoisomer, geometrical isomer, polymorph, hydrate, solvate, metabolite, and prodrug thereof, for the preparation of a medicament for the treatment of a condition or disorder selected from prostate cancer, rectal cancer, renal cancer, ovarian cancer, endometrial cancer, thyroid cancer, pancreatic cancer, breast cancer, colon cancer, bladder cancer, brain cancer, glial cancer, melanoma cancer, pineal gland cancer, or lung cancer.

These and other features, aspects, and advantages of the present subject matter will become better understood with reference to the following description. This summary is provided to introduce a selection of concepts in a simplified form. This summary is not intended to identify key features or essential features of the disclosure, nor is it intended to be used to limit the scope of the subject matter.

### DETAILED DESCRIPTION

Those skilled in the art will be aware that the present disclosure is subject to variations and modifications other than those specifically described. It is to be understood that the present disclosure includes all such variations and modifications. The disclosure also includes all such steps, features, compositions and compounds referred to or indicated in this specification, individually or collectively, and any and all combinations of any or more of such steps or features.

### Definitions

For convenience, before further description of the present disclosure, certain terms employed in the specification, and examples are collected here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles“a”, “an” and“the” are used to refer to one or to more than one (i.e., to at least one) of the grammatical object of the article.

Throughout the description and the claims which follow, unless the context requires otherwise, the word “comprise”, and variations such as “comprises” and“comprising”, will be understood to imply the inclusion of a stated integer or step or group of integers but not to the exclusion of any other integer or step or group of integers or steps.

The term“including” is used to mean“including but not limited to”. “Including” and“including but not limited to” are used interchangeably.

In the structural formulae given herein and throughout the present disclosure, the following terms have been indicated meaning, unless specifically stated otherwise.

The term “alkyl” refers to a monoradical branched or unbranched saturated hydrocarbon chain having 1, 2, 3, 4, 5, 6, 7, 8, 9, 10, 11, 12, 13, 14, 15, 16, 17, 18, 19 or 20 carbon atoms, preferably 1, 2, 3, 4, 5, 6, 7, 8, 9 or 10 carbon atoms, more preferably 1, 2, 3, 4, 5 or 6 carbon atoms. This term is exemplified by groups such as methyl, ethyl, n-propyl, iso-propyl, n-butyl, iso-butyl, t-butyl, n-hexyl, n-decyl, tetradecyl, and the like.

The term “alkylene” refers to a diradical of a branched or unbranched saturated hydrocarbon chain, having 1, 2, 3, 4, 5, 6, 7, 8, 9,10, 11,12, 13, 14, 15, 16, 17, 18, 19 or 20 carbon atoms, preferably 1, 2, 3, 4, 5, 6, 7, 8, 9 or 10 carbon atoms, more preferably 1, 2, 3, 4, 5 or 6 carbon atoms. This term is exemplified by groups such as methylene (—CH2—), ethylene (—CH2CH2—), the propylene isomers (e.g., —CH2CH2CH2— and —CH(CH3)CH2—) and the like.

The term “substituted alkyl” or“substituted alkylene” refers to:
1) an alkyl group or alkylene group as defined above, having 1, 2, 3, 4 or 5 substituents, preferably 1, 2 or 3 substituents, selected from the group consisting of alkenyl, alkynyl, alkoxy, cycloalkyl, cycloalkenyl, acyl, acylamino, acyloxy, amino, monoalkylamino, dialkylamino, arylamino, heteroarylamino, aminocarbonyl, alkoxycarbonylamino, azido, cyano, halogen, hydroxy, hydroxyalkyl, keto, thiocarbonyl, carboxy, carboxyalkyl, —S03 H, aryl, aryloxy, heteroaryl, aminocarbonylamino, heteroaryloxy, heterocyclyl, heterocyclyloxy, hydroxyamino, alkoxyamino, nitro, —S(0)2 NRa Ra, —NRa S(0)2 Ra and —S(0)p Rb, where each Ra is independently selected from the group consisting of hydrogen, alkyl, cycloalkyl, cycloalkylalkyl, aryl, arylalkyl, heteroaryl heteroarylalkyl, heterocyclyl and heterocyclylalkyl; heterocyclyloxy where Rb is hydrogen, alkyl, aryl, heteroaryl or heterocyclyl. Unless otherwise constrained by the definition, all substituents may optionally be further substituted by 1, 2, or 3 substituents selected from alkyl, carboxy, carboxyalkyl, aminocarbonyl, hydroxy, alkoxy, halogen, CF3, amino, substituted amino, cyano, and —S(0)p Rc, where Rc is alkyl, aryl, or heteroaryl and p is 0,1 or 2;
or 2) an alkyl group or alkylene group as defined above that is interrupted by 1, 2, 3, 4, 5, 6, 7, 8, 9 or 10 atoms independently selected from oxygen, sulfur and NRd , where Rd is selected from hydrogen, alkyl, cycloalkyl, cycloalkenyl, aryl, heteroaryl and heterocyclyl, carbonylalkyl, carboxyester, carboxyamide and sulfonyl. All substituents may be optionally further substituted by alkyl, alkoxy, halogen, CF3, amino, substituted amino, cyano, or —S(0)p Rc, in which Rc is alkyl, aryl, or heteroaryl and p is 0, 1, or 2;
or 3) an alkyl or alkylene as defined above that has 1, 2, 3, 4 or 5 substituents as defined above, as well as interrupted by 1, 2, 3, 4, 5, 6, 7, 8, 9 or 10 atoms as defined above.

The term “alkenyl” refers to a monoradical of a branched or unbranched unsaturated hydrocarbon group preferably having from 2, 3, 4, 5, 6, 7, 8, 9, 10, 11, 12, 13, 14, 15, 16, 17, 18, 19 or 20 carbon atoms, more preferably 2, 3, 4, 5, 6, 7, 8, 9 or 10 carbon atoms and even more preferably 2, 3, 4, 5 or 6 carbon atoms and having 1, 2, 3, 4, 5 or 6 double bond (vinyl), preferably 1 double bond. Preferred alkenyl groups include ethenyl or vinyl(—CH═CH2), 1- propylene or allyl (—CH2 CH═CH2), isopropylene (—C(CH3)═CH2), bicyclo [2.2. 1] heptene, and the like. The term “alkenylene” refers to a diradical of a branched or unbranched unsaturated hydrocarbon group preferably having from 2, 3, 4, 5, 6, 7, 8, 9, 10, 11, 12, 13, 14, 15, 16, 17, 18, 19 or 20 carbon atoms, more preferably 2, 3, 4, 5, 6, 7, 8, 9 or 10 carbon atoms and even more preferably 2, 3, 4, 5 or 6 carbon atoms and having 1, 3, 4, 5 or 6 double bond (vinyl), preferably 1 double bond.

The term “substituted alkenyl” refers to an alkenyl group as defined above having 1, 2, 3, 4 or 5 substituents, and preferably 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, thiocarbonyl, carboxy, carboxyalkyl, —SO3 H, aryl, aryloxy, heteroaryl, aminocarbonylamino, heteroaryloxy, heterocyclyl, heterocyclyloxy, hydroxyamino, alkoxyamino, nitro, —S(0)2 NRa Ra, —NRa S(0)2 Ra and —S(0)p Rb where each Ra is independently selected from the group consisting of hydrogen, alkyl, cycloalkyl, cycloalkylalkyl, aryl, arylalkyl, heteroaryl heteroarylalkyl, heterocyclyl and heterocyclylalkyl; heterocyclyloxywhere Rb is alkyl, aryl, heteroaryl or heterocyclyl and p is 0, 1 or 2. Unless otherwise constrained by the definition, all substituents may optionally be further substituted by 1, 2, or 3 substituents selected from alkyl, carboxy, carboxyalkyl, aminocarbonyl, hydroxy, alkoxy, halogen, CF3, amino, substituted amino, cyano, and —S(0)p Rc, where Rc is alkyl, aryl, or heteroaryl and p is 0, 1 or 2.

The term “alkynyl” refers to a monoradical of an unsaturated hydrocarbon, preferably having from 2, 3, 4, 5, 6, 7, 8, 9, 10, 11, 12, 13, 14, 15, 16, 17, 18, 19 or 20 carbon atoms, more preferably 2, 3, 4, 5, 6, 7, 8, 9 or 10 carbon atoms and even more preferably 2, 3, 4, 5 or 6 carbon atoms and having 1, 2, 3, 4, 5 or 6 sites of acetylene (triple bond) unsaturation, preferably 1 triple bond. Preferred alkynyl groups include ethynyl, (—C° CH), propargyl (or prop-1-yn-3-yl, —CH2 C° CH), homopropargyl (or but-1-yn-4-yl, —CH2 CH2 C° CH) and the like.

The term “alkynylene” refers to a diradical of a branched or unbranched unsaturated hydrocarbon group preferably having from 2, 3, 4, 5, 6, 7, 8, 9, 10, 11, 12, 13, 14, 15, 16, 17, 18, 19 or 20 carbon atoms, more preferably 2, 3, 4, 5, 6, 7, 8, 9 or 10 carbon atoms and even more preferably 2, 3, 4, 5 or 6 carbon atoms and having 1, 3, 4, 5 or 6 sites of acetylene (triple bond) unsaturation, preferably 1 triple bond.

The term “substituted alkynyl” refers to an alkynyl group as defined above having 1, 2, 3, 4 or 5 substituents, and preferably 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, —S03 H, aryl, aryloxy, heteroaryl, aminocarbonylamino, heteroaryloxy, heterocyclyl, heterocyclyloxy, hydroxyamino, alkoxyamino, nitro, —S(0)2 NRa Ra, —NRa S(0)2 Ra and —S(0)p Rb, where each Ra is independently selected from the group consisting of hydrogen, alkyl, cycloalkyl, cycloalkylalkyl, aryl, arylalkyl, heteroaryl heteroarylalkyl, heterocyclyl and heterocyclylalkyl; heterocyclyloxy where Rb is alkyl, aryl, heteroaryl or heterocyclyl and p is 0, 1 or 2. Unless otherwise constrained by the definition, all substituents may optionally be further substituted by 1, 2, or 3 substituents selected from alkyl, carboxy, carboxyalkyl, aminocarbonyl, hydroxy, alkoxy, halogen, CF3, amino, substituted amino, cyano, and —S(0)p Rc where Rc is alkyl, aryl, or heteroaryl and p is 0, 1 or 2.

The term “cycloalkyl” refers to carbocyclic groups of from 3 to 20 carbon atoms having a single cyclic ring or multiple condensed rings which may be partially unsaturated. Such cycloalkyl groups include, by way of example, single ring structures such as cyclopropyl, cyclobutyl, cyclopentyl, cyclopentenyl, cyclohexyl, cyclohexenyl, cyclooctyl, and the like, or multiple ring structures such as adamantanyl, bicyclo[2.2.1]heptane, 1,3,3-trimethylbicyclo[2.2.1]hept-2-yl, (2,3,3-trimethylbicyclo[2.2.1]hept-2-yl), or carbocyclic groups to which is fused an aryl group, for example indane, and the like.

The term “substituted cycloalkyl” refers to cycloalkyl groups having 1, 2, 3, 4 or 5 substituents, and preferably 1, 2, or 3 substituents, selected from the group consisting of alkyl, alkoxy, cycloalkyl, cycloalkenyl, acyl, acylamino, acyloxy, amino, aminocarbonyl, alkoxycarbonylamino, azido, cyano, halogen, hydroxy, oxo, thiocarbonyl, aryl, aryloxy, heteroaryl, aminosulfonyl, aminocarbonylamino, heteroaryloxy, heterocyclyl, heterocyclyloxy, hydroxyamino, alkoxyamino, nitro, —C(0)R and —S(0)p Rb, where R is hydrogen, hydroxyl, alkoxy, alkyl and cyclocalkyl, heterocyclyloxy where Rb is alkyl, aryl, heteroaryl or heterocyclyl and p is 0, 1 or 2. Unless otherwise constrained by the definition, all substituents may optionally be further substituted by 1, 2, or 3 substituents selected from alkyl, carboxy, carboxyalkyl, aminocarbonyl, hydroxy, alkoxy, halogen, CF3, amino, substituted amino, cyano, and —S(0)p Rc, where Rc is alkyl, aryl, or heteroaryl and p is 0, 1 or 2.

“Halo” or“Halogen”, alone or in combination with any other term means halogens such as chloro (CI), fluoro (F), bromo (Br) and iodo (I).

“Haloalkyl” refers to a straight chain or branched chain haloalkyl group with 1 to 6 carbon atoms. The alkyl group may be partly or totally halogenated. Representative examples of haloalkyl groups include but are not limited to fluoromethyl, chloromethyl, bromomethyl, difluoromethyl, dichloromethyl, dibromomethyl, trifluoromethyl, trichloromethyl, 2-fluorocthyl, 2-chlorocthyl, 2-bromocthyl, 2,2,2-trifluoroethyl, 3-fluoropropyl, 3-chloropropyl, 3-bromopropyl and the like.

The term “alkoxy” refers to the group R″-0-, where R′″ is optionally substituted alkyl or optionally substituted cycloalkyl, or optionally substituted alkenyl or optionally substituted alkynyl; or optionally substituted cycloalkenyl, where alkyl, alkenyl, alkynyl, cycloalkyl and cycloalkenyl are as defined herein. Representative examples of alkoxy groups include but are not limited to methoxy, ethoxy, n-propoxy, iso-propoxy, n-butoxy, tert-butoxy, sec-butoxy, n-pentoxy, n-hexoxy, 1,2-dimethylbutoxy, trifluoromethoxy, and the like.

The term “aminocarbonyl” refers to the group —C(0)NR′R′ where each R′ is independently hydrogen, alkyl, aryl, heteroaryl, heterocyclyl or both R′ groups are joined to form a heterocyclic group (e. g. morpholino). Unless otherwise constrained by the definition, all substituents may optionally be further substituted by 1-3 substituents selected from alkyl, carboxy, carboxyalkyl, aminocarbonyl, hydroxy, alkoxy, halogen, CF3, amino, substituted amino, cyano, and —S(0)p Rc, where Rc is alkyl, aryl, or heteroaryl and p is 0, 1 or 2. The term “acylamino” refers to the group —NR″C(0)R″ where each R″ is independently hydrogen, alkyl, aryl, heteroaryl, or heterocyclyl. Unless otherwise constrained by the definition, all substituents may optionally be further substituted by 1-3 substituents selected from alkyl, carboxy, carboxyalkyl, aminocarbonyl, hydroxy, alkoxy, halogen, CF3, amino, substituted amino, cyano, and —S(0)p Rc, where Rc is alkyl, aryl, or heteroaryl and p is 0, 1 or 2.

The term “acyloxy” refers to the groups —OC(0)-alkyl, —OC(0)-cycloalkyl, —OC(0)-aryl, —OC(0)-heteroaryl, and —OC(0)-heterocyclyl. Unless otherwise constrained by the definition, all substituents may be optionally further substituted by alkyl, carboxy, carboxyalkyl, aminocarbonyl, hydroxy, alkoxy, halogen, CF3, amino, substituted amino, cyano, or —S(0)p Rc, where Rc is alkyl, aryl, or heteroaryl and p is 0, 1 or 2.

The term“alkoxyalkyl” refers to alkyl groups as defined above wherein at least one of the hydrogen atoms of the alkyl group is replaced by an alkoxy group as defined above. Representative examples of alkoxyalkyl groups include but are not limited to methoxymethyl, methoxyethyl, ethoxymethyl and the like.

The term“aryloxyalkyl” refers to the group-alkyl-O-aryl. Representative examples of aryloxyalkyl include but are not limited to phenoxymethyl, naphthyloxymethyl, phenox yethyl, naphthyloxyethyl and the like.

The term“di alkylamino” refers to an amino group, to which two same or different straight chain or branched chain alkyl groups with 1 to 6 carbon atoms are bound. Representative examples of di alkylamino include but are not limited to dimethylamino, diethylamino, methylethylamino, dipropylamino, dibutylamino and the like.

The term“cycloalkylalkyl” refers to an alkyl radical as defined above which is substituted by a cycloalkyl radical as defined above. Representative examples of cycloalkylalkyl include but are not limited to cyclopropylmethyl, cyclobutylmethyl, cyclopentylmethyl, cyclohexylmethyl, 1-cyclopentylethyl, 1-cyclohexylethyl, 2-cyclopentylethyl, 2-cyclohexylethyl, cyclobutylpropyl,

cyclopentylpropyl, cyclohexylbutyl and the like. The term“aminoalkyl” refers to an amino group that is attached to (Ci-ejalkylene as defined herein. Representative examples of aminoalkyl include but are not limited to aminomethyl, aminoethyl, l-aminopropyl, 2-aminopropyl, and the like. The amino moiety of aminoalkyl may be substituted once or twice with alkyl to provide alkylaminoalkyl and dialkylaminoalkyl respectively. Representative examples of alkylaminoalkyl include but are not limited to methylaminomethyl, methylaminocthyl, methylaminopropyl, ethylaminoethyl and the like. Representative examples of dialkylaminoalkyl include but are not limited to dimethylaminomethyl, dimethylaminoethyl, dimethylaminopropyl, N-methyl-N-cthylaminoethyl and the like.

The term “aryl” refers to an aromatic carbocyclic group of 6 to 20 carbon atoms having a single ring (e.g. phenyl) or multiple rings (e.g. biphenyl), or multiple condensed (fused) rings (e.g. naphthyl or anthranyl). Preferred aryls include phenyl, naphthyl and the like.

The term “arylene” refers to a diradical of an aryl group as defined above. This term is exemplified by groups such as 1,4-phenylene, 1,3-phenylene, 1,2-phenylene, 1,4′-biphenylene, and the like.

Unless otherwise constrained the aryl or arylene groups may optionally be substituted with 1, 2, 3 4 or 5 substituents, preferably 1, 2 or 3 substituents, selected from the group consisting of alkyl, alkoxy, cycloalkyl, cycloalkenyl, acyl, acylamino, acyloxy, amino, aminocarbonyl, alkoxycarbonylamino, azido, cyano, halogen, hydroxy, carboxy, carboxyalkyl, —S03 H, aryl, aryloxy, heteroaryl, aminosulfonyl, aminocarbonylamino, heteroaryloxy, heterocyclyl, heterocyclyloxy, hydroxyamino, alkoxyamino, nitro, —S(0)2 NRa Ra, —NRa S(0)2 Ra and —S(0)p Rb where each Ra is independently selected from the group consisting of hydrogen, alkyl, cycloalkyl, cycloalkylalkyl, aryl, arylalkyl, heteroaryl, heteroarylalkyl, heterocyclyl and heterocyclylalkyl; where Rb is hydrogen, alkyl, aryl, heterocyclyl or heteroaryl and p is 0, 1 or 2. Unless otherwise constrained by the definition, all substituents may optionally be further substituted by 1, 2 or 3 substituents selected from alkyl, carboxy, carboxyalkyl, aminocarbonyl, hydroxy, alkoxy, halogen, CF3, amino, substituted amino, cyano, and —S(0)p Rc where Rc is hydrogen, alkyl, aryl, or heteroaryl and p is 0, 1 or 2.

The term “arylalkyl” refers to an aryl group covalently linked to an alkylene group, where aryl and alkylene are defined herein.

The term “optionally substituted arylalkyl” refers to an optionally substituted aryl group covalently linked to an optionally substituted alkylene group. Such arylalkyl groups are exemplified by benzyl, phenethyl, naphthylmethyl, and the like.

The term “aryloxy” refers to the group-O-aryl, wherein the aryl group is as defined above and includes optionally substituted aryl groups as also defined above.

The term “arylthio” refers to the group—S-aryl, where aryl group is as defined herein including optionally substituted aryl groups as also defined above.

The term “substituted amino” refers to the group —NR′R′ where each R′is independently selected from the group consisting of hydrogen, alkyl, cycloalkyl, carboxyalkyl, alkoxycarbonyl, aryl, heteroaryl and heterocyclyl. Unless otherwise constrained by the definition, all substituents may optionally be further substituted by 1, 2 or 3 substituents selected from alkyl, carboxy, carboxyalkyl, aminocarbonyl, hydroxy, alkoxy, halogen, CF3, amino, substituted amino, cyano, and —S(0)p Rc, where Rc is alkyl, aryl, or heteroaryl and p is 0, 1 or 2.

The term “carboxyalkyl” refers to the group-alkylene—C(0)OH.

The term “ alky lcarboxy alkyl” refers to the group-alkylene—C(0)ORd where Rd is alkyl, cycloalkyl, where alkyl, cycloalkyl are as defined herein, and may be optionally further substituted by alkyl, halogen, CF3, amino, substituted amino, cyano, or —S(0)p Rc, in which Rc is alkyl, aryl, or heteroaryl and p is 0, 1 or 2.

The term “heteroaryl” refers to an aromatic cyclic group having 1, 2, 3, 4, 5, 6, 7, 8, 9, 10, 11, 12, 13, 14, or 15 carbon atoms and 1, 2, 3 or 4 heteroatoms selected from oxygen, nitrogen and sulfur within at least one ring. Such heteroaryl groups can have a single ring (e.g. pyridyl or furyl) or multiple condensed rings (e.g. indolizinyl, benzothiazolyl, or benzothienyl). Examples of heteroaryls include, but are not limited to, [1,2,4] oxadiazole, [1,3,4] oxadiazole, [1,2,4] thiadiazole, [1,3,4] thiadiazole, pyrrole, imidazole, pyrazole, pyridine, pyrazine, pyrimidine, pyridazine, indolizine, isoindole, indole, indazole, purine, quinolizine, isoquinoline, quinoline, phthalazine, quinoxaline, quinazoline, cinnoline, pteridine, carbazole, carboline, phenanthridine, acridine, phenanthroline, isothiazole, phenazine, isoxazole, phenoxazine, phenothiazine, furan, thiophene, oxazole, thiazole, triazole, triazine and the like.

The term “heteroarylene” refers to a diradical of a heteroaryl group as defined above.

Unless otherwise constrained the heteroaryl or heterarylene groups can be optionally substituted with 1, 2, 3, 4 or 5 substituents, preferably 1, 2 or 3 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, thiocarbonyl, carboxy, carboxyalkyl, —S03 H, aryl, aryloxy, heteroaryl, aminocarbonylamino, heteroaryloxy, heterocyclyl, heterocyclyloxy, hydroxyamino, alkoxyamino, nitro, —S(0)2 NRa Ra, —NRa S(0)2 Ra and —S(0)p Rb, where each Ra is independently selected from the group consisting of hydrogen, alkyl, cycloalkyl, cycloalkylalkyl, aryl, arylalkyl, heteroaryl heteroarylalkyl, heterocyclyl and heterocyclylalkyl; where Rb is hydrogen, alkyl, aryl, heterocyclyl or heteroaryl, and p is 0, 1 or 2. Unless otherwise constrained by the definition, all substituents may optionally be further substituted by 1-3 substituents selected from alkyl, carboxy, carboxyalkyl, aminocarbonyl, hydroxy, alkoxy, halogen, CF3, amino, substituted amino, cyano, and —S(0)n Rc, where Rc is alkyl, aryl, or heteroaryl and n is 0,1 or 2.

The term “heteroarylalkyl” refers to a heteroaryl group covalently linked to an alkylene group, where heteroaryl and alkylene are defined herein.

The term “optionally substituted heteroarylalkyl” refers to an optionally substituted heteroaryl group covalently linked to an optionally substituted alkylene group. Such heteroarylalkyl groups are exemplified by 3-pyridylmethyl, quinolin-8-ylethyl, 4-methoxythiazol-2-ylpropyl, and the like.

The term “heterocyclyl” refers to a saturated or partially unsaturated group having a single ring or multiple condensed rings, having from 1 to 40 carbon atoms and from 1 to 10 hetero atoms, preferably 1, 2, 3 or 4 heteroatoms, selected from nitrogen, sulfur, phosphorus, and/or oxygen within the ring. Heterocyclic groups can have a single ring or multiple condensed rings, and include tetrahydrofuranyl, morpholinyl, piperidinyl, piperazinyl, dihydropyridinyl, tetrahydroquinolinyl and the like. Unless otherwise constrained by the definition for the heterocyclic substituent, such heterocyclic groups can be optionally substituted with 1, 2, 3, 4 or 5, and preferably 1, 2 or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, oxo, —C(0)R where R is hydrogen, hydroxyl, alkoxy, alkyl and cyclocalkyl, thiocarbonyl, carboxy, carboxyalkyl, aryl, aryloxy, heteroaryl, aminosulfonyl, aminocarbonylamino, heteroaryloxy, heterocyclyl, heterocyclyloxy, hydroxyamino, alkoxyamino, nitro, and —S(0)p Rb, where Rb is hydrogen, alkyl, aryl, heterocyclyl or heteroaryl and p is 0, 1 or 2. Unless otherwise constrained by the definition, all substituents may optionally be further substituted by 1-3 substituents selected from alkyl, carboxy, carboxyalkyl, aminocarbonyl, hydroxy, alkoxy, halogen, CF3, amino, substituted amino, cyano, and —S(0)Rc, where Rc is alkyl, aryl, or heteroaryl and n is 0, 1 or 2.

The term “heterocyclylalkyl” refers to a heterocyclyl group covalently linked to an alkylene group, where heterocyclyl and alkylene are defined herein.

The term “optionally substituted heterocyclylalkyl” refers to an optionally substituted heterocyclyl group covalently linked to an optionally substituted alkylene group.

The term “heteroaryloxy” refers to the group-O-heteroaryl.

The term “thiol” refers to the group —SH.

The term “substituted alkylthio” refers to the group —S-substituted alkyl.

The term “heteroarylthio” refers to the group —S-heteroaryl wherein the heteroaryl group is as defined above including optionally substituted heteroaryl groups as also defined above.

The term “sulfoxide” refers to a group —S(O). The term “substituted sulfoxide” refers to a group —S(0)R, in which R is substituted alkyl, substituted aryl, or substituted heteroaryl, as defined above.

The term “sulfone” refers to a group —S(0)2 R, where R is alkyl, aryl, or heteroaryl.

The term“substituted sulfone” refers to a group —S(0)2 R, in which R is alkyl, aryl, or heteroaryl.

The term “disorder or condition ameliorated by the inhibition of the A2 A receptor” will be understood by those skilled in the art to include: cancer such as prostate, rectal, renal, ovarian, endometrial, thyroid, pancreatic, particularly breast, colon, bladder, brain, glia, melanoma, pincal gland and, more particularly, lung cancer (e.g. Lewis lung carcinoma).

The compounds of the present disclosure may have the ability to crystallize in more than one form, a characteristic known as polymorphism, and all such polymorphic forms (“polymorphs”) are encompassed within the scope of the invention. Polymorphism generally can occur as a response to changes in temperature or pressure or both and can also result from variations in the crystallization process. Polymorphs can be distinguished by various physical characteristics, and typically the x-ray diffraction patterns, solubility behavior, and melting point of the compound are used to distinguish polymorphs.

The compounds described herein may contain one or more chiral centers and/or double bonds and therefore, may exist as“stereoisomers”, such as double bond isomers (i.e., “geometric isomers”), regioisomers, enantiomers or diastereomers. Accordingly, the chemical structures depicted hercin encompass all possible enantiomers and stereoisomers of the illustrated or identified compounds including the stereoisomerically pure form (e.g., geometrically pure, enantiomerically pure or diastercomerically pure) and enantiomeric and stereoisomeric mixtures. Enantiomeric and stercoisomeric mixtures can be resolved into their component enantiomers or stereoisomers using separation techniques or chiral synthesis techniques well known to the person skilled in the art. The compounds may also exist in several tautomeric forms including the enol form, the keto form and mixtures thereof.

Accordingly, the chemical structures depicted herein encompass all possible tautomeric forms of the illustrated or identified compounds.

Compounds may exist in unsolvated forms as well as solvated forms, including hydrated forms and as N-oxides. In general, compounds may be hydrated, solvated or N-oxides. Certain compounds may exist in multiple crystalline or amorphous forms. Also contemplated within the scope of the invention are congeners, analogs, hydrolysis products, metabolites and precursor or prodrugs of the compound. In general, unless otherwise indicated, all physical forms are equivalent for the uses contemplated herein and are intended to be within the scope of the present invention.

The term“prodrug” refers to a derivative of a drug molecule as, for example, esters, carbonates, carbamates, ureas, amides or phosphates that requires a transformation within the body to release the active drug. Prodrugs are frequently, although not necessarily, pharmacologically inactive until converted to the parent drug. Prodrugs may be obtained by bonding a promoicty (defined herein) typically via a functional group, to a drug.

The term “therapeutically effective dose” means an amount of a compound or composition which is sufficient enough to significantly and positively modify the symptoms and/or conditions to be 10 treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, the route of administration, and like factors within the knowledge 15 and expertise of the attending physician.

The term“promoiety” refers to a group bonded to a drug, typically to a functional group of the drug, via bond(s) that are cleavable under specified conditions of use. The bond(s) between the drug and promoicty may be cleaved by enzymatic or non-enzymatic means. Under the conditions of use, for example following administration to a patient, the bond(s) between the drug and promoiety may be cleaved to release the parent drug. The cleavage of the promoicty may proceed spontaneously, such as via a hydrolysis reaction, or it may be catalyzed or induced by another agent, such as by an enzyme, by light, by acid, or by a change of or exposure to a physical or environmental parameter, such as a change of temperature, pH, etc. The agent may be endogenous to the conditions of use, such as an enzyme present in the systemic circulation to which the prodrug is administered or the acidic conditions of the stomach or the agent may be supplied exogenously.

The phrase“pharmaceutically acceptable excipient” refers to compounds or compositions that are physiologically tolerable and do not typically produce allergic or similar untoward reactions, including but not limited to gastric upset or dizziness when administered to mammal.

The term “pharmaceutically acceptable salt” embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali carth metal (e.g. calcium or magnesium) hydroxides and organic bases, for example alkyl amines, arylalkyl amines and heterocyclic amines.

Other preferred salts according to the invention are quaternary ammonium compounds wherein an equivalent of an anion (X-) is associated with the positive charge of the N atom. X-may be an anion of various mineral acids such as, for example, chloride, bromide, iodide, sulphate, nitrate, phosphate, or an anion of an organic acid such as, for example, acetate, malcate, fumarate, citrate, oxalate, succinate, tartrate, malate, mandelate, trifluoroacetate, methanesulphonate and p-toluenesulphonate. X-is preferably an anion selected from chloride, bromide, iodide, sulphate, nitrate, acetate, maleate, oxalate, succinate or trifluoroacetate. More

preferably X-is chloride, bromide, trifluoroacetate or methanesulphonate. Furthermore, the compound of Formula I, Formula II, Formula III, or Formula IV can be its derivatives, analogs, stereoisomer's, diastereomers, geometrical isomers, polymorphs, solvates, co-crystals, intermediates, hydrates, metabolites, prodrugs or pharmaceutically acceptable salts and compositions.

It is understood that included in the family of compounds of Formula I, Formula II, Formula III, or Formula IV are isomeric forms including diastercoisomers, enantiomers, tautomers, and geometrical isomers in“E” or“Z” configurational isomer or a mixture of E and Z isomers. It is also understood that some isomeric forms such as diastercomers, enantiomers and geometrical isomers can be separated by physical and/or chemical methods by those skilled in the art.

Compounds disclosed herein may exist as single stereoisomers, racemates and or mixtures of enantiomers and/or diastercomers. All such single stereoisomers, racemates and mixtures thereof are intended to be within the scope of the subject matter described.

Compounds disclosed herein include isotopes of hydrogen, carbon, oxygen, fluorine, chlorine, iodine and sulfur which can be incorporated into the compounds, such as, but not limited to,2 H (D),3 H (T), U C,13 C,14 C,15 N,18 F,35 S,36 Cl, and125 I. Compounds of this disclosure wherein atoms were isotopically labeled for example radioisotopes such as3 H,13 C,14 C, and the like can be used in metabolic studies, kinetic studies, and imaging techniques such as positron emission tomography used in understanding the tissue distribution of the drugs. Compounds of the disclosure where hydrogen is replaced with deuterium may improve the metabolic stability, and pharmacokinetics properties of the drug such as in vivo half-life.

The pharmaceutical composition comprising compounds of Formula I, Formula II, Formula III, or Formula IV and their analogs, tautomeric forms, stercoisomers, geometrical isomers, polymorphs, hydrates, solvates, pharmaceutically acceptable salts, pharmaceutical compositions, metabolites, and prodrugs thereof can also be referred as“composition of the present disclosure”.

In an embodiment of the present disclosure, there is provided a pharmaceutical composition comprising compound of Formula I and its pharmaceutically acceptable salt, analog, tautomeric form, stereoisomer, geometrical isomer, polymorph, hydrate, solvate, metabolite, and prodrug thereof

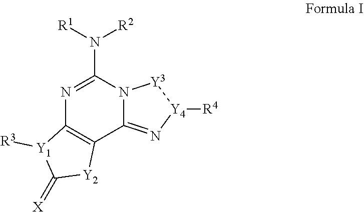

wherein
represents a single bond or a double bond;
X is selected from O, S or NRa; Yi is selected from N or CH; Y2 is selected from NR5, O or CR5 R6;
Y3 is selected from N, CH, CH2, C(=0), or C(═S); Y4 is selected from N, C, or CH; R1 and R2 are independently selected from hydrogen or alkyl; R3 is -A-Z-B-Q; wherein, A is absent or is a group selected from alkylene, alkenylene, or alkynylene; wherein one or more methylene groups is optionally replaced by hetero atoms or groups such as -0-, —S(0)p-, —N(Ra)—, or —C(O); alkylene, alkenylene and alkynylene is optionally substituted with—(CRd Re)n OR7, (CRd Re) n COOR7, —(CRd Re)n NR8 R9, cyano, halogen, haloalkyl, perhaloalkyl, alkoxyalkoxy, alkyl, or cycloalkyl;
Z is absent or is selected from a cycloalkyl or a heterocyclyl; wherein cycloalkyl and heterocyclyl are unsubstituted or substituted independently with 1, 2, or 3 substituents independently selected from alkyl, alkenyl, alkynyl, acyl, —(CRd Re)n OR7, (CRd Re)n COOR7, —(CRd Re)n NR8 R9, aminocarbonyl, alkoxycarbonylamino, halogen, haloalkyl, perhaloalkyl, azido, cyano, keto, thiocarbonyl, —S03 H, aminocarbonylamino, nitro, —S(0)2 NRa Ra, —NRb S(0)2 Rb or —S(0)P Rc;
B is absent or is a group selected from alkylene, alkenylene or alkynylene; wherein one or more methylene groups is optionally replaced by hetero atoms or groups such as -0-, —S(0)p-, —N(Ra)—, or —C(O); alkylene, alkenylene and alkynylene is optionally substituted with hydroxy, amino, aminoalkyl, cyano, halogen, haloalkyl, perhaloalkyl, carboxy, carboxyalkyl, alkoxy, hydroxyalkyl, alkoxyalkyl, alkoxyalkoxy or alkyl;
Q is selected from hydrogen, alkyl, cycloalkyl, cycloalkylalkyl, aryl, arylalkyl, heterocyclyl, heterocyclylalkyl, heteroaryl or heteroarylalkyl; wherein alkyl, cycloalkyl, cycloalkylalkyl, aryl, arylalkyl, heterocyclyl, heterocyclylalkyl, heteroaryl and heteroarylalkyl are unsubstituted or substituted independently with 1, 2, or 3 substituents independently selected from alkyl, alkenyl, alkynyl, halogen, haloalkyl, perhaloalkyl, azido, cyano, nitro, keto, thiocarbonyl, cyanoalkyl, cyanoalkylcarbonyl, —(CRd Re)n OR7, —(CRd Re)n C(0)R7, —(CRd Re)n SR7, —(CRd Re)n COOR7, —(CRd Re)n NR8 R9, —(CRd Re)n C(0)NR8 R9, —(CRd Re)n NR8 C(0)OR7, —(CRd Re), NR8 C(0)NR8 R9, —NRb S(0)2 Rb, —S(0)P Rc, —S03 H, —S(0)2 NRa Ra, cycloalkyl, cycloalkenyl, cycloalkylalkyl aryl, arylalkyl, heterocyclyl, heterocyclylalkyl, heteroaryl or heteroarylalkyl; wherein each substituent is unsubstituted or substituted with 1, 2, or 3 substituents independently selected from alkyl, carboxy, carboxyalkyl, aminocarbonyl, hydroxy, alkoxy, halogen, haloalkyl, perhaloalkyl, haloalkoxy, perhaloalkoxy, amino, substituted amino, cyano or —S(0)P Rc;
R4 is selected from the group consisting of hydrogen, alkyl, alkenyl, alkynyl, haloalkyl, hydroxyalkyl, carboxyalkyl, cycloalkyl, cycloalkylalkyl, aryl, arylalkyl, heterocyclyl, heterocyclylalkyl, heteroaryl and heteroarylalkyl; wherein alkyl, alkenyl, alkynyl, cycloalkyl, cycloalkylalkyl, arylalkyl, aryl, heteroaryl, heteroarylalkyl, heterocyclyl, and heterocyclylalkyl are unsubstituted or substituted independently with up to four substituents independently selected from alkyl, alkenyl, alkynyl, acyl, —(CRd Re)n OR7, (CRd Re)n COOR7, —(CRd Re)n NR8 R9, aminocarbonyl, alkoxycarbonylamino, aminocarbonylamino, azido, cyano, halogen, haloalkyl, perhaloalkyl, keto, nitro, —S(0)2 NRb Rb, —NRb S(0)2 Rb or —S(0)p Rc, thiocarbonyl, —SO3H, cycloalkyl, cycloalkenyl, aryl, heteroaryl or heterocyclyl;
R5 and R6 are independently selected from the group consisting of hydrogen, hydroxy, —(CRd Re) n OR7, (CRd Re)n COOR7, —(CRd Re)n NR8 R9, cyanoalkyl, haloalkyl, alkoxyalkoxyalkyl, alkyl, alkenyl, alkynyl, cycloalkyl, cycloalkylalkyl, aryl, arylalkyl, heterocyclyl, heterocyclylalkyl, heteroaryl and heteroarylalkyl;
R7 is selected from hydrogen, alkyl, halogen, haloalkyl, —(CRd Re)n OR7, —(CRd Re)n COOR7, -(CRe Re)n C(0)R7, carbonylamino, cycloalkyl, cycloalkylalkyl, aryl, arylalkyl, heteroaryl, heteroarylalkyl, heterocyclyl or heterocyclylalkyl;
R8 and R9 are independently selected from the group consisting of hydrogen, alkyl, haloalkyl, —(CRd Re)n OR7, —(CRd Re)n C(0)R7, aryl, arylalkyl, heteroaryl, heteroarylalkyl, cycloalkyl, cycloalkylalkyl, heterocyclyl and heterocyclylalkyl, or R8 and R9 taken together form a monocyclic or a bicyclic ring system which is saturated or partially unsaturated and optionally have additional heteroatoms selected from O, N or S, said ring system is further optionally substituted with 1 to 4 substituents independently selected from halo, alkyl, alkenyl, alkynyl, nitro, cyano, —(CRd Re)n OR7, —(CRd Re)n SR7, —(CRd Re)n NR8 R9, oxo, alkylsulfonyl, —(CRd Rc)n COOR7, —(CRd Re)n C(0)NR8 R9, cycloalkyl, cycloalkylalkyl, aryl, arylalkyl, heterocyclyl, heterocyclylalkyl, heteroaryl, or heteroarylalkyl;
Ra is selected from hydrogen or alkyl; Rb cach is independently selected from the group consisting of hydrogen, alkyl, acyl, carboxyalkyl, carbonylamino, cycloalkyl, cycloalkylalkyl, aryl, arylalkyl, heteroaryl, heteroarylalkyl, heterocyclyl and heterocyclylalkyl; Rc is selected from alkyl, cycloalkyl, aryl, heterocyclyl or heteroaryl; Rd and Re are independently selected from the group consisting of hydrogen, —OR7, halogen, haloalkyl, perhaloalkyl and alkyl;
n is 0, 1, 2, 3 or 4, and
p is 0, 1 or 2,
for the manufacture of a medicament for the treatment of a condition or disorder ameliorated by inhibition of the A2A/A2B receptor.

In an embodiment of the present disclosure, there is provided a pharmaceutical composition comprising compound of Formula I and its pharmaceutically acceptable salt, analog, tautomeric form, stereoisomer, geometrical isomer, polymorph, hydrate, solvate, metabolite, and prodrug thereof, wherein
represents a double bond; X is selected from O, S or NRa;
Y i is selected from N or CH;
Y2 is selected from NR5 or CR5 R6;
Y3 is selected from N, CH or CH2;
Y4 is selected from N or C;
R1 and R2 are independently selected from hydrogen or alkyl;
R3 is -A-Z-B-Q;
wherein, A is absent or is alkylene wherein one or more methylene groups is optionally replaced by hetero atoms or groups selected from the group consisting of-O-, —S(0)p-, —N(Ra)—, or —C(O); alkylene is optionally substituted with—(CRd Re),OR7, cyano, halogen, haloalkyl, perhaloalkyl, alkyl or cycloalkyl;
Z is absent or is selected from a cycloalkyl or a heterocyclyl;
wherein cycloalkyl and heterocyclyl are unsubstituted or substituted independently with 1, 2, or 3 substituents independently selected from alkyl, acyl, —(CRd Re)n OR7, (CRd Re)n COOR7, —(CRd Re)n NR8 R9, aminocarbonyl, alkoxycarbonylamino, halogen, haloalkyl, perhaloalkyl, azido, cyano, halogen, keto, thiocarbonyl, —S03 H, aminocarbonylamino, nitro, —S(0)2 NRa Ra, —NRb S(0)2 Rb or —S(0)P Rc;
B is absent or is alkylene wherein one or more methylene groups is optionally replaced by hetero atoms or groups selected from the group consisting of-O-, —S(0)p-, —N(Ra)—, or —C(O); alkylene is optionally substituted with hydroxy, amino, aminoalkyl, cyano, halogen, haloalkyl, perhaloalkyl, carboxy, carboxyalkyl, alkoxy, hydroxyalkyl, alkoxyalkyl, alkoxyalkoxy or alkyl;
Q is selected from hydrogen, alkyl, cycloalkyl, cycloalkylalkyl, aryl, arylalkyl, heterocyclyl, heterocyclylalkyl, heteroaryl or heteroarylalkyl;
wherein alkyl, cycloalkyl, cycloalkylalkyl, aryl, arylalkyl, heterocyclyl, heterocyclylalkyl, heteroaryl and heteroarylalkyl are unsubstituted or substituted independently with 1, 2, or 3 substituents independently selected from alkyl, alkenyl, alkynyl, alkoxy, alkoxyalkyl, haloalkyl, perhaloalkyl, azido, cyano, nitro, halogen, keto, thiocarbonyl, cyanoalkyl, cyanoalkylcarbonyl, —(CRd Re)n OR7, —(CRd Re),C(0)R7, —(CRd Re),SR7, —(CRd Re), COOR7, —(CRd Rc),NR8 R9, —(CRd Re)n C(0)NR8 R9, —(CRd Re)n NR8 C(0)OR7, —(CRd Re),NR8 C(0)NR8 R9, —NRb S(0)2 Rb, —S(0)P Rc, —S03 H, —S(0)2 NRa Ra, cycloalkyl, cycloalkenyl, cycloalkylalkyl aryl, arylalkyl, heterocyclyl, heterocyclylalkyl, heteroaryl or heteroarylalkyl;
wherein cach substituent is unsubstituted or substituted with 1, 2, or 3 substituents independently selected from alkyl, carboxy, carboxyalkyl, aminocarbonyl, hydroxy, alkoxy, halogen, haloalkyl, perhaloalkyl, haloalkoxy, perhaloalkoxy, amino, substituted amino, cyano or —S(0)p Rc;
R4 is selected from the group consisting of hydrogen, alkyl, haloalkyl, hydroxyalkyl, carboxyalkyl, cycloalkyl, cycloalkylalkyl, aryl, arylalkyl, heterocyclyl, heterocyclylalkyl, heteroaryl and heteroarylalkyl;
wherein alkyl, cycloalkyl, cycloalkylalkyl, arylalkyl, aryl, heteroaryl, heteroarylalkyl, heterocyclyl, and heterocyclylalkyl are unsubstituted or independently substituted with up to four substituents independently selected from alkyl, acyl, —(CRd Re)n OR7, (CRd Re)n COOR7, —(CRd Re)n NR8 R9, aminocarbonyl, alkoxycarbonylamino, aminocarbonylamino, azido, cyano, halogen, haloalkyl, perhaloalkyl, keto, nitro, —S(0)2 NRb Rb, —NRb S(0)2 Rb or —S(0)p Rc, thiocarbonyl, —S03 H, cycloalkyl, cycloalkenyl, aryl, heteroaryl or heterocyclyl;
R5 and R6 are independently selected from the group consisting of hydrogen, hydroxy, —(CRd Rc)n OR7, (CRd Rc)n COOR7, —(CRd Re)n NR8 R9, cyanoalkyl, haloalkyl, alkoxyalkoxyalkyl, alkyl, alkenyl, alkynyl, cycloalkyl, cycloalkylalkyl, aryl, arylalkyl, heterocyclyl, heterocyclylalkyl, heteroaryl or heteroarylalkyl;
R7 is selected from hydrogen, alkyl, halogen, haloalkyl, carbonylamino, cycloalkyl, cycloalkylalkyl, aryl, arylalkyl, heteroaryl, heteroarylalkyl, heterocyclyl or heterocyclylalkyl;
R8 and R9 are independently selected from the group consisting of hydrogen, alkyl, haloalkyl, —(CRd Re)n OR7, —(CRd Re)n C(0)R7, aryl, arylalkyl, heteroaryl, heteroarylalkyl, cycloalkyl, cycloalkylalkyl, hctcrocyclyl and heterocyclylalkyl, or R8 and R9 taken together form a monocyclic or a bicyclic ring system which is saturated or partially unsaturated and optionally have additional heteroatoms selected from O, N or S, said ring system is further optionally substituted with 1 to 4 substituents independently selected from halo, alkyl, alkenyl, alkynyl, nitro, cyano, —(CRd Re)n OR7, —(CRd Re)n SR7, —(CRd Re)n NR8 R9, oxo, alkylsulfonyl, —(CRd Re)n COOR7, —(CRd Re)n C(0)NR8 R9, cycloalkyl, cycloalkylalkyl, aryl, arylalkyl, heterocyclyl, heterocyclylalkyl, heteroaryl, or heteroarylalkyl;
Ra is selected from hydrogen or alkyl;
Rb each is independently selected from the group consisting of hydrogen, alkyl, acyl, carboxyalkyl, carbonylamino, cycloalkyl, cycloalkylalkyl, aryl, arylalkyl, hctcroaryl, heteroarylalkyl, heterocyclyl and heterocyclylalkyl;
Rc is selected from alkyl, cycloalkyl, aryl, heterocyclyl or heteroaryl;
Rd and Re are independently selected from the group consisting of hydrogen, —OR7, halogen, haloalkyl, perhaloalkyl and alkyl;
n is 0, 1, 2, 3 or 4 and
p is 0, 1 or 2, for the manufacture of a medicament for the treatment of a condition or disorder ameliorated by inhibition of the A2A/A2B receptor.

In an embodiment of the present disclosure, there is provided a pharmaceutical composition comprising compound of Formula I and its pharmaceutically acceptable salt, analog, tautomeric form, stereoisomer, geometrical isomer, polymorph, hydrate, solvate, metabolite, and prodrug thereof, wherein
represents a double bond;
X is selected from O or S;
Yi represents N;
Y2 represents NR5;
Y3 represents N;
Y4 represents C;
R1 and R2 are independently selected from hydrogen or alkyl;
R3 is -A-Z-B-Q;
wherein, A is absent or is alkylene wherein one or more methylene groups is optionally replaced by hetero atoms or groups selected from the group consisting of-O-, —S(0)p-, —N(Ra)—, or —C(O);
Z is absent or is a heterocyclyl; wherein the heterocyclyl is unsubstituted or substituted independently with 1, 2, or 3 substituents independently selected from alkyl, acyl, —(CRd Re)n OR7, (CRd Re)n COOR7, —(CRd Re)n NR8 R9, haloalkyl, perhaloalkyl, cyano, halogen, keto, thiocarbonyl, —S03 H, nitro, —S(0)2 NRa Ra, —NRb S(0)2 Rb or —S(0)P Rc;
B is absent or is alkylene wherein one or more methylene groups is optionally replaced by hetero atoms or groups selected from the group consisting of-O-, - S(0)p-, —N(Ra)—, or —C(O);
Q is selected from hydrogen, alkyl, cycloalkyl, aryl, heterocyclyl, or heteroaryl; wherein alkyl, cycloalkyl, aryl, heterocyclyl and heteroaryl are unsubstituted or independently substituted with 1, 2, or 3 substituents independently selected from alkyl, alkoxy, alkoxyalkyl, haloalkyl, perhaloalkyl, azido, cyano, nitro, halogen, keto, thiocarbonyl, cyanoalkyl, cyanoalkylcarbonyl, —(CRd Re)n OR7, —(CRd Re),C(0)R7, —(CRd Re), SR7, —(CRd Re), COOR7, -

(CRd Re),NR8 R9, —(CRd Re)n C(0)NR8 R9, —(CRd Re)n NR8 C(0)OR7, —(CRd Re)n NR8 C(0)NR8 R9, —NRb S(0)2 Rb, —S(0)pRc, —SO3H, —S(0)2 NRa Ra, cycloalkyl, cycloalkenyl, cycloalkylalkyl aryl, arylalkyl, heterocyclyl, heterocyclylalkyl, heteroaryl or heteroarylalkyl;
wherein cach substituent is unsubstituted or substituted with 1, 2, or 3 substituents independently selected from alkyl, carboxy, carboxyalkyl, aminocarbonyl, hydroxy, alkoxy, halogen, haloalkyl, perhaloalkyl, haloalkoxy, perhaloalkoxy, amino, substituted amino, cyano or —S(0)p Rc;

R4 is selected from the group consisting of hydrogen, alkyl, haloalkyl, hydroxyalkyl, carboxyalkyl, cycloalkyl, cycloalkylalkyl, aryl, arylalkyl, heterocyclyl, heterocyclylalkyl, heteroaryl and heteroarylalkyl;
wherein alkyl, cycloalkyl, cycloalkylalkyl, arylalkyl, aryl, heteroaryl, heteroarylalkyl, heterocyclyl, and heterocyclylalkyl are unsubstituted or independently substituted with up to four substituents independently selected from alkyl, acyl, —(CRd Re)n OR7, (CRd Re)n COOR7, —(CRd Re)n NR8 R9, aminocarbonyl, alkoxycarbonylamino, aminocarbonylamino, azido, cyano, halogen, haloalkyl, perhaloalkyl, keto, nitro, —S(0)2 NRb Rb, —NRb S(0)2 Rb or —S(0)p Rc, thiocarbonyl, —SO3 H, cycloalkyl, cycloalkenyl, aryl, heteroaryl or heterocyclyl; R5 and R6 are independently selected from the group consisting of hydrogen, hydroxy, —(CRd Re) n OR7, (CRd Rc)n COOR7, —(CRd Re)n NR8 R9, cyanoalkyl, haloalkyl, alkoxyalkoxyalkyl, alkyl, alkenyl, alkynyl, cycloalkyl, cycloalkylalkyl, aryl, arylalkyl, heterocyclyl, heterocyclylalkyl, heteroaryl and heteroarylalkyl;
R7 is selected from hydrogen, alkyl, halogen, haloalkyl, carbonylamino, cycloalkyl, cycloalkylalkyl, aryl, arylalkyl, heteroaryl, heteroarylalkyl, heterocyclyl or heterocyclylalkyl;

R8 and R9 are independently selected from the group consisting of hydrogen, alkyl, haloalkyl, —(CRd Re)n OR7, —(CRd Re)n C(0)R7, aryl, arylalkyl, heteroaryl, heteroarylalkyl, cycloalkyl, cycloalkylalkyl, heterocyclyl and heterocyclylalkyl, or R8 and R9 taken together form a monocyclic or a bicyclic ring system which is saturated or partially unsaturated and optionally have additional heteroatoms selected from O, N or S, said ring system is further optionally substituted with 1 to 4 substituents independently selected from halo, alkyl, alkenyl, alkynyl, nitro, cyano, —(CRd Re)n OR7, —(CRd Re)n SR7, —(CRd Re)n NR8 R9, oxo, alkylsulfonyl, —(CRd Re)n COOR7, —(CRd Re)n C(0)NR8 R9, cycloalkyl, cycloalkylalkyl, aryl, arylalkyl, heterocyclyl, heterocyclylalkyl, heteroaryl, or heteroarylalkyl;
Ra is selected from hydrogen or alkyl;
Rb cach is independently selected from the group consisting of hydrogen, alkyl, acyl, carboxyalkyl, carbonylamino, cycloalkyl, cycloalkylalkyl, aryl, arylalkyl, heteroaryl, heteroarylalkyl, heterocyclyl and heterocyclylalkyl;
Rc is selected from alkyl, cycloalkyl, aryl, heterocyclyl or heteroaryl;
Rd and Re are independently selected from the group consisting of hydrogen, —OR7, halogen, haloalkyl, perhaloalkyl or alkyl;
n is 0, 1, 2, 3 or 4 and
p is 0, 1 or 2, for the manufacture of a medicament for the treatment of a condition or disorder ameliorated by inhibition of the A2A/A2B receptor.

In an embodiment of the present disclosure, there is provided a pharmaceutical composition comprising compound of Formula I and its pharmaceutically acceptable salt, analog, tautomeric form, stereoisomer, geometrical isomer, polymorph, hydrate, solvate, metabolite, and prodrug thereof, wherein
represents a double bond;
X selected from O or S;
Yi represents N;
Y2 represents NR5;
Y3 represents N;
Y4 represents C;
R1 and R2 are independently selected from hydrogen or alkyl;
R3 is -A-Z-B-Q;
wherein, A is absent or is alkylene wherein one or more methylene groups is optionally replaced by hetero atoms or groups selected from the group consisting of —O— or —N(Ra)—;
Z is absent or is a heterocyclyl;
wherein the heterocyclyl is unsubstituted or substituted independently with 1, 2, or 3 substituents independently selected from alkyl, —(CRd Re)n OR7, (CRd Re)n COOR7, haloalkyl, perhaloalkyl, cyano, halogen, keto or thiocarbonyl;
B is absent or is alkylene wherein one or more methylene groups is optionally replaced by hetero atoms or groups selected from the group consisting of-O-, —N(Ra)—, or —C(O);
Q is selected from hydrogen, alkyl, cycloalkyl, aryl, heterocyclyl, or heteroaryl; wherein alkyl, cycloalkyl, aryl, heterocyclyl and heteroaryl are unsubstituted or independently substituted with 1, 2, or 3 substituents independently selected from alkyl, alkoxy, alkoxyalkyl, haloalkyl, perhaloalkyl, cyano, halogen, keto, thiocarbonyl, cyanoalkyl, —(CRd Re)n OR7, —(CRd Re)n C(0)R7, —(CRd Re)n COOR7, —(CRd Re)n NR8 R9, —(CRd Re), C(0)NR8 R9, —(CRd Re), NR8 C(0)OR7, —S(0)P Rc, —S03 H, —S(0)2 NRa Ra, cycloalkyl, cycloalkenyl, aryl, heterocyclyl or heteroaryl; wherein each substituent is unsubstituted or substituted with 1, 2, or 3 substituents independently selected from alkyl, carboxy, carboxyalkyl, aminocarbonyl, hydroxy, alkoxy, halogen, haloalkyl, perhaloalkyl, haloalkoxy, perhaloalkoxy, amino, substituted amino, cyano or —S(0)p Rc; R4 is selected from the group consisting of hydrogen, alkyl, haloalkyl, hydroxyalkyl, cycloalkyl, aryl, heterocyclyl and heteroaryl;
wherein alkyl, cycloalkyl, aryl, heteroaryl and heterocyclyl are unsubstituted or independently substituted with up to four substituents independently selected from alkyl, —(CRd Re)n OR7, (CRd Re)n COOR7, —(CRd Re)n NR8 R9, cyano, halogen, haloalkyl, perhaloalkyl, nitro, —S(0)2 NRb Rb, —NRb S(0)2 Rb, —S(0)p Rc, thiocarbonyl, —S03 H, cycloalkyl, aryl, heteroaryl or heterocyclyl;
R5 is selected from the group consisting of hydrogen, hydroxy, haloalkyl, —(CRd Re)n OR7, —(CRd Re)n COOR7, alkoxyalkoxyalkyl, alkyl, alkenyl, alkynyl, cycloalkyl, cycloalkylalkyl, aryl, arylalkyl, heterocyclyl, heterocyclylalkyl, heteroaryl or heteroarylalkyl;
R7 is selected from hydrogen, alkyl, halogen, haloalkyl, cycloalkyl, aryl, heteroaryl or heterocyclyl;
R8 and R9 are independently selected from the group consisting of hydrogen, alkyl, haloalkyl, —(CRd Re)n OR7, —(CRd Re)n C(0)R7, aryl, arylalkyl, heteroaryl, heteroarylalkyl, cycloalkyl, cycloalkylalkyl, heterocyclyl and heterocyclylalkyl, or R8 and R9 taken together form a monocyclic or a bicyclic ring system which is saturated or partially unsaturated and optionally have additional heteroatoms selected from O, N or S, said ring system is further optionally substituted with 1 to 4 substituents independently selected from halo, alkyl, nitro, cyano, —(CRd Re)n OR7, —(CRd Re)n NR8 R9, oxo, alkylsulfonyl, —(CRd Re)n COOR7 or —(CRd Re),C(0)NR8 R9;
Ra is selected from hydrogen or alkyl;
Rb each is independently selected from the group consisting of hydrogen, alkyl, acyl, carboxyalkyl, carbonylamino, cycloalkyl, cycloalkylalkyl, aryl, arylalkyl, heteroaryl, heteroarylalkyl, heterocyclyl or heterocyclylalkyl;
Rc is selected from alkyl, cycloalkyl, aryl, heterocyclyl or heteroaryl;
Rd and Re are independently selected from the group consisting of hydrogen, —OR7, halogen, haloalkyl, perhaloalkyl and alkyl;
n is 0, 1, 2, 3 or 4 and p is 0, 1 or 2, for the manufacture of a medicament for the treatment of a condition or disorder ameliorated by inhibition of the A2A/A2B receptor.

In an embodiment of the present disclosure, there is provided a pharmaceutical composition comprising compound of Formula I and its pharmaceutically acceptable salt, analog, tautomeric form, stereoisomer, geometrical isomer, polymorph, hydrate, solvate, metabolite, and prodrug thereof, wherein
represents a double bond;
X is selected from O or S;
Yi represents N;
Y2 represents NR5;
Y3 represents N;
4 represents C;
R1 and R2 are independently selected from hydrogen or alkyl;
R3 is -A-Z-B-Q;
wherein, A is absent or is alkylene wherein one or more methylene groups is optionally replaced by hetero atoms or groups selected from the group consisting of —O— or —N(Ra)—;
Z is absent or is a heterocyclyl selected from dihydrofuranyl, tetrahydrofuranyl, morpholinyl, pyrrolidinyl, dihydropyrrole, dihydropyranyl, tetrahydropyranyl, pyrazolidinyl, imidazolidinyl, dihydropyridinyl, tetrahydropyridinyl, piperidinyl, dihydropyrazinyl, tetrahydropyrazinyl, piperazinyl or dihydropyridinyl;
wherein the heterocyclyl is unsubstituted or substituted independently with 1, 2, or 3 substituents independently selected from alkyl, —(CRd Re)n OR7,
(CRd Rc)n COOR7, haloalkyl, perhaloalkyl, cyano or halogen;
B is absent or is alkylene wherein one or more methylene groups is optionally replaced by hetero atoms or groups selected from the group consisting of —O—, —N(Ra)—, or —C(O);
Q is selected from hydrogen, alkyl, cyclopropyl, cyclopentyl, cyclohexyl, phenyl, tetrahydrofuranyl, pyrrolidinyl, tetrahydropyridinyl, tetrahydropyranyl, piperazinyl, benzodiaxolyl, tetrahydroquinolinyl, morpholinyl, tetrahydronaphthyridinyl, tetrahydrothicnopyridinyl, furanyl, pyridinyl, pyrimidinyl, oxazolyl, thiazolyl, oxadiazolyl, thiadiazolyl, indolyl, quinolinyl, isoquinolinyl or benzooxazolyl;
wherein Q is unsubstituted or substituted with 1, 2, or 3 substituents independently selected from alkyl, alkoxy, alkoxyalkyl, haloalkyl, perhaloalkyl, cyano, halogen, keto, thiocarbonyl, cyanoalkyl, —(CRd Re)n OR7, —(CRd Re)n C(0)R7, —(CRd Re)n COOR7, —(CRd Re)n NR8 R9, —(CRd Re)n C(0)NR8 R9, —(CRd Re),NR8 C(0)OR7, —S(0)P Rc, —S03 H, —S(0)2 NRa Ra, cycloalkyl, cycloalkenyl, aryl, heterocyclyl or heteroaryl;
wherein each substituent is unsubstituted or substituted with 1, 2, or 3 substituents independently selected from alkyl, carboxy, carboxyalkyl, aminocarbonyl, hydroxy, alkoxy, halogen, haloalkyl, perhaloalkyl, haloalkoxy, perhaloalkoxy, amino, substituted amino, cyano or —S(0)p Rc;
R4 is selected from the group consisting of hydrogen, alkyl, phenyl, naphthyl, furanyl, thiazolyl, oxazolyl, thiadiazolyl, oxadiazolyl, pyrazinyl, pyridinyl and pyrimidinyl;
wherein R4 is unsubstituted or substituted with up to four substituents independently selected from alkyl, —(CRd Re)n OR7, (CRd Re)n COOR7, —(CRd Re)n NR8 R9, cyano, halogen, haloalkyl, perhaloalkyl or cycloalkyl;
R5 is selected from the group consisting of hydrogen, hydroxy, haloalkyl, —(CRd Re)n OR7, —(CRd Re)n COOR7, alkoxyalkoxyalkyl, alkyl, alkonyl, alkynyl, cycloalkyl, cycloalkylalkyl, aryl, arylalkyl, heterocyclyl, heterocyclylalkyl, heteroaryl and heteroarylalkyl;
R7 is selected from hydrogen, alkyl, halogen, haloalkyl, cycloalkyl, aryl, heteroaryl or heterocyclyl;
R8 and R9 are independently selected from the group consisting of hydrogen, alkyl, haloalkyl, —(CRd Re)n OR7, —(CRd Re)n C(0)R7, aryl, arylalkyl, heteroaryl, heteroarylalkyl, cycloalkyl, cycloalkylalkyl, heterocyclyl and heterocyclylalkyl, or R8 and R9 taken together form a monocyclic or a bicyclic ring system which is saturated or partially unsaturated and optionally have additional heteroatoms selected from O, N or S, said ring system is further optionally substituted with 1 to 4 substituents independently selected from halo, alkyl, nitro, cyano, —(CRd Re)n OR7, —(CRd Re)n NR8 R9, oxo, alkylsulfonyl, —(CRd Re)n COOR7 or—(CRd Re), C(0)NR8 R9;
Ra is selected from hydrogen or alkyl;
Rb at cach occurrence is independently selected from the group consisting of hydrogen, alkyl, acyl, carboxyalkyl, carbonylamino, cycloalkyl, cycloalkylalkyl, aryl, arylalkyl, heteroaryl, heteroarylalkyl, heterocyclyl or heterocyclylalkyl;
Rc is selected from alkyl, cycloalkyl, aryl, heterocyclyl or heteroaryl;
Rd and Re are independently selected from the group consisting of hydrogen, —OR7, halogen, haloalkyl, perhaloalkyl and alkyl;
n is 0, 1, 2, 3 or 4 and
p is 0, 1 or 2, for the manufacture of a medicament for the treatment of a condition or disorder ameliorated by inhibition of the A2A/A2B receptor.

In an embodiment of the present disclosure, there is provided a pharmaceutical composition comprising compound of Formula I and its pharmaceutically acceptable salt, analog, tautomeric form, stercoisomer, geometrical isomer, polymorph, hydrate, solvate, metabolite, and prodrug thereof as disclosed herein, for use in the treatment of a condition or disorder selected from prostate cancer, rectal cancer, renal cancer, ovarian cancer, endometrial cancer, thyroid cancer, pancreatic cancer, breast cancer, colon cancer, bladder cancer, brain cancer, glial cancer, melanoma cancer, pineal gland cancer, or lung cancer.

In an embodiment of the present disclosure, there is provided a method of using the pharmaceutical composition comprising compound of Formula I and its pharmaceutically acceptable salt, analog, tautomeric form, stercoisomer, geometrical isomer, polymorph, hydrate, solvate, metabolite, and prodrug thereof as disclosed herein, in the treatment of a disease or condition in a mammal that is amenable to treatment with an A2 A/A2B receptor antagonist, the method comprising: administering to a mammal in need thereof a therapeutically effective dose of the pharmaceutical composition comprising compound of Formula I, its pharmaceutically acceptable salt, analog, tautomeric form, stercoisomer, geometrical isomer, polymorph, hydrate, solvate, metabolite, and prodrug thereof.

In an embodiment of the present disclosure, there is provided a method of treatment of a disorder or condition ameliorated by antagonizing the A2 A/A2B receptor, the method comprising: administering an effective amount of the pharmaceutical composition comprising compound of Formula I, its pharmaceutically acceptable salt, analog, tautomeric form, stereoisomer, geometrical isomer, polymorph, hydrate, solvate, metabolite, and prodrug thercof as disclosed herein to a patient in need of such treatment.

In an embodiment of the present disclosure, there is provided a use of the pharmaceutical composition comprising compound of Formula I, its pharmaceutically acceptable salt, analog, tautomeric form, stereoisomer, geometrical isomer, polymorph, hydrate, solvate, metabolite, and prodrug thereof as disclosed herein, for the preparation of a medicament for the treatment of a condition or disorder selected from prostate cancer, rectal cancer, renal cancer, ovarian cancer, endometrial cancer, thyroid cancer, pancreatic cancer, breast cancer, colon cancer, bladder cancer, brain cancer, glial cancer, melanoma cancer, pineal gland cancer, or lung cancer.

In an embodiment of the present disclosure, there is provided a pharmaceutical composition comprising compound of Formula I, its pharmaceutically acceptable salt, analog, tautomeric form, stereoisomer, geometrical isomer, polymorph, hydrate, solvate, metabolite, and prodrug thereof as disclosed herein, in combination with at least one PD-L1 antibody for use in the treatment of a condition or disorder selected from prostate cancer, rectal cancer, renal cancer, ovarian cancer, endometrial cancer, thyroid cancer, pancreatic cancer, breast cancer, colon cancer, bladder cancer, brain cancer, glial cancer, melanoma cancer, pineal gland cancer, or lung cancer.

In an embodiment of the present disclosure, there is provided a use of the pharmaceutical composition comprising compound of Formula I, its pharmaceutically acceptable salt, analog, tautomeric form, stereoisomer, geometrical isomer, polymorph, hydrate, solvate, metabolite, and prodrug thereof as disclosed herein, in combination with at least one PD-L1 antibody for use in the treatment of a condition or disorder selected from prostate cancer, rectal cancer, renal cancer, ovarian cancer, endometrial cancer, thyroid cancer, pancreatic cancer, breast cancer, colon cancer, bladder cancer, brain cancer, glial cancer, melanoma cancer, pineal gland cancer, or lung cancer.

In an embodiment of the present disclosure, there is provided a pharmaceutical composition comprising compound of Formula I, its pharmaceutically acceptable salt, analog, tautomeric form, stereoisomer, geometrical isomer, polymorph, hydrate, solvate, metabolite, and prodrug thereof as disclosed herein, wherein the compound of Formula I is selected from the group consisting of:
5-Amino-8-(2-furyl)-3-[2-[4-[4-(2-methoxyethoxy)phenyl|pipcrazin-1-yl]ethyl]-1-methyl-[1,2,4] triazolo [5, 1-f]purin-2-one (1),
5-Amino-8-(2-furyl)-3-(2-hydroxyethyl)-1-methyl-[1,2,4]triazolo[5,1-f]purin-2-one (2),
5-Amino-3-[2-[4-(2,4-difluorophenyl)piperazin-1-yl]ethyl]-8-(2-furyl)-1-methyl-[1,2,4]triazolo[5, 1-f]purin-2-one (3),
5-Amino-8-(2-furyl)-3-[2-[4-(4-methoxyphenyl)piperazin-1-yl]ethyl]-1-methyl-[1,2,4]triazolo[5, 1-f]purin-2-one (4),
5-Amino-8-(2-furyl)-1-methyl-3-(2-morpholinoethyl)-[1,2,4]triazolo[5,1-f]purin-2-one (5),
5-Amino-3-[2-[4-(2,4-difluorophenyl)-1-piperidyl]ethyl]-8-(2-furyl)-1-methyl-[1,2,4]triazolo[5, 1-f]purin-2-one (6),
5-Amino-8-(2-furyl)-1-methyl-3-[2-[4-(5-methyl-2-pyridyl)piperazin-1-yl]ethyl]-[1,2,4]triazolo[5, 1-f]purin-2-one (7),
5-Amino-8-(2-furyl)-1-methyl-3-[2-[4-(p-tolyl)piperazin-1-yl]ethyl]-[1,2,4]triazolo[5, 1-f]purin-2-one (8),
5-Amino-8-(2-furyl)-1-methyl-3-[2-[4-(3-methyl-2-oxo-butyl)piperazin-1-yl]ethyl]-[1,2,4]triazolo[5,1-f]purin-2-one (9),
5-Amino-3-[2-[4-(2-fluoro-4-methoxy-phenyl)piperazin-1-yl]ethyl]-8-(2-furyl)-1-methyl-[1,2,4] triazolo [5, 1-f]purin-2-one (10),
5-Amino-8-(2-furyl)-3-[2-[4-[4-(2-methoxy-1, 1-dimethyl-ethoxy)phenyl] piperazin-1-yl] ethyl]-1-methyl-[1,2,4] triazolo[5, 1-f]purin-2-one (11), 5-Amino-8-(2-furyl)-3-[2-[4-(6-methoxy-3-pyridyl)piperazin-1-yl]ethyl]-1-methyl-[1,2,4]triazolo[5, 1-f]purin-2-one (12),
5-Amino-3-[2-[4-[3-fluoro-4-(2-methoxyethoxy)phenyl]piperazin-1-yl]ethyl]-8-(2-furyl)-1-methyl-[1,2,4]triazolo[5,1-f]purin-2-one (13),
5-Amino-8-(2-furyl)-3-[2-[4-[4-(1-hydroxy-1-methyl-ethyl)phenyl]piperazin-1-yl]ethyl]-1-methyl-[1,2,4]triazolo[5,1-f]purin-2-one (14),
5-Amino-3-[2-[4-(4-fluorophenyl)-4-hydroxy-1-piperidyl]ethyl]-8-(2-furyl)-1-methyl-[1,2,4] triazolo [5, 1-f]purin-2-one (15),
5-Amino-8-(2-furyl)-3-[2-[4-[4-(2-methoxy-2-methyl-propoxy)phenyl]piperazin-1-yl]ethyl]-1-methyl-[1,2,4]triazolo[5,1-f]purin-2-one (16),
5-Amino-3-[2-[4-[4-(cyclopropoxy)phenyl]piperazin-1-yl]ethyl]-8-(2-furyl)-1-methyl-[1,2,4] triazolo [5, 1-f]purin-2-one (17),
5-Amino-3-[2-[4-(4-fluorophenyl)-3,6-dihydro-2H-pyridin-1-yl]ethyl]-8-(2-furyl)-1-methyl-[1,2,4] triazolo [5, 1-f]purin-2-one (18),
5-Amino-8-(2-furyl)-3-[2-[4-hydroxy-4-(4-methoxyphenyl)-1-piperidyl]ethyl]-1-methyl-[1,2,4] triazolo [5, 1-f]purin-2-one (19),
5-Amino-3-[2-[4-[3 ,5-difluoro-4-(2-methoxyethoxy)phenyl]piperazin-1-yl]ethyl]-8-(2-furyl)-1-methyl-[1,2,4]triazolo[5,1-f]purin-2-one (20),
5-Amino-3-[2-[4-[2,5-difluoro-4-(2-methoxyethoxy)phenyl]piperazin-1-yl]ethyl]-8-(2-furyl)-1-methyl-[1,2,4]triazolo[5,1-f]purin-2-one (21),
5-Amino-3-[2-[4-(2,2-difluoro-1,3-benzodioxol-5-yl)piperazin-1-yl]ethyl]-8-(2-furyl)-1-methyl-[1,2,4] triazolo [5, 1-f]purin-2-one (22),
5-Amino-8-(2-furyl)-3-[2-[4-[4-(2-methoxyethoxy)phenyl]-3,3-dimethyl-piperazin-1-yl]ethyl]-1-methyl-[1,2,4] triazolo[5, 1-f]purin-2-one (23),
5-Amino-3-[2-(4-butylpiperazin-1-yl)ethyl]-8-(2-furyl)-1-methyl-[1,2,4]triazolo[5, 1-f]purin-2-one (24),
5-Amino-8-(2-furyl)-3-[2-(4-hydroxy-4-methyl-1-piperidyl)ethyl]-1-methyl-[1,2,4]triazolo[5, 1-f]purin-2-one (25),
5-Amino-3-[2-[4-[4-[2-(cyclopropoxy)ethoxy]phenyl]piperazin-1-yl]ethyl]-8-(2-furyl)-1-methyl-[1,2,4]triazolo[5,1-f]purin-2-one (26), 5-Amino-8-(2-furyl)-3-[2-[4-[(4-methoxyphenyl)methyl]piperazin-1-yl]ethyl]-1-methyl-[1,2,4] triazolo [5, 1-f]purin-2-one (27),
5-Amino-8-(2-furyl)-3-[2-[4-[[4-(2-methoxyethoxy)phenyl] methyl]piperazin-1-yl]ethyl]-1-methyl-[1,2,4]triazolo[5,1-f]purin-2-one (28),
5-Amino-8-(2-furyl)-3-[(4-methoxyphenyl)methyl]-1-methyl-[1,2,4] triazolo [5,1-f]purin-2-one (29),
5-Amino-8-(2-furyl)-3-[2-[4-(4-methoxyphenyl)piperazin-1-yl]ethyl]-1-methyl-[1,2,4]triazolo[5, 1-f]purin-2-one (30),
5-Amino-8-(2-furyl)-3-[2-[4-[3-(2-methoxyethoxy)phenyl]piperazin-1-yl]ethyl]-1-methyl-[1,2,4]triazolo[5,1-f]purin-2-one (31),
5-Amino-3-[2-[4-[2-fluoro-4-(2-methoxyethoxy)phenyl]piperazin-1-yl]ethyl]-8-(2-furyl)-1-methyl-[1,2,4] triazolo [5, 1-f]purin-2-one (32), 4-[4-[2-[5-Amino-8-(2-furyl)-1-methyl-2-oxo-[1,2,4]triazolo[5,1-f]purin-3 y1] ethyl] piperazin-1-yl] benzonitrile (33), 4-[4-[2-[5-Amino-8-(2-furyl)-1-methyl-2-oxo-[1,2,4]triazolo[5,1-f]purin-3-yl]ethyl]piperazin-1-y1]-2-fluoro-benzonitrile (34),
5-Amino-8-(2-furyl)-1-methyl-3-[2-[4-[4-(trifluoromethyl)phenyl]piperazin-1-yl]ethyl]-[1,2,4]triazolo[5,1-f]purin-2-one (35),
5-Amino-8-(2-furyl)-1-methyl-3-[2-[4-[4-(trifluoromethyl)thiazol-2-yl]piperazin-1-yl]ethyl]-[1,2,4]triazolo[5,1-f]purin-2-one (36),
5-Amino-3-[2-[4-(cyclopropylmethyl)piperazin-1-yl]ethyl]-8-(2-furyl)-1-methyl-[1,2,4]triazolo[5, 1-f]purin-2-one (37),
5-Amino-3-[2-(4-ethylpiperazin-1-yl)ethyl]-8-(2-furyl)-1-methyl-[1,2,4]triazolo[5, 1-f]purin-2-one (38), 4-[2-[5-Amino-8-(2-furyl)-1-methyl-2-oxo-[1,2,4]triazolo[5,1-f]purin-3-yl]ethyl]-N,N-dimethyl -piperazine-1-sulfonamide (39),
5-Amino-8-(2-furyl)-1-methyl-3-[2-[4-(4-tetrahydrofuran-3-yloxyphenyl)piperazin-1-yl]ethyl] -[1,2,4] triazolo[5, 1-f]purin-2-one (40),
5-Amino-8-(2-furyl)-1-methyl-3-[2-[4-(4-tetrahydropyran-4-yloxyphenyl)piperazin-1-yl]ethyl] -[1,2,4] triazolo[5, 1-f]purin-2-one (41),
5-Amino-8-(2-furyl)-1-methyl-3-[2-[4-[4-(tetrahydrofuran-2-ylmethoxy)phenyl]piperazin-1-yl]ethyl]-[1,2,4]triazolo[5,1-f]purin-2-one (42),
5-Amino-8-(2-furyl)-1-methyl-3-[2-(3-methyl-7,8-dihydro-5H-1,6-naphthyridin-6-yl)ethyl]-[1,2,4]triazolo[5,1-f]purin-2-one (43),
5-Amino-3-[2-(6,7-dihydro-4H-thieno[3,2-c]pyridin-5-yl)ethyl]-8-(2-furyl)-1-methyl-[1,2,4] triazolo [5, 1-f]purin-2-one (44),
5-Amino-8-(2-furyl)-3-[2-[4-[4-(2-methoxyethoxy)phenyl]piperazin-1-yl]propyl]-1-methyl-[1,2,4] triazolo [5, 1-f]purin-2-one (45),
5-Amino-3-[2-[3-(4-fluorophenyl)-2,5-dihydropyrrol-1-yl]ethyl]-8-(2-furyl)-1-methyl-[1,2,4]triazolo[5,1-f]purin-2-one (46),
5-Amino-3-[2-[4-[4-(2-methoxyethoxy)phenyl]piperazin-1-yl]ethyl]-1-methyl-8-(5-methyl-2-furyl)-[1,2,4]triazolo[5,1-f]purin-2-one (47),
5-Amino-8-(5-cyclopropyl-2-furyl)-3-[2-[4-[4-(2-methoxyethoxy)phenyl]piperazin-1-yl]ethyl] -1-methyl-[1,2,4] triazolo[5, 1-f]purin-2-one (48),
5-Amino-3-[2-(2,4-difluoroanilino)ethyl]-8-(2-furyl)-1-methyl-[1,2,4]triazolo[5,1-f]purin-2-one (49),
5-Amino-3-[3-[4-(4-fluorophenyl)piperazin-1-yl]propyl]-8-(2-furyl)-1-methyl-[1,2,4]triazolo[5, 1-f]purin-2-one (50),
5-Amino-8-(2-furyl)-3-[3-[4-[4-(2-methoxyethoxy)phenyl]piperazin-1-yl]propyl]-1-methyl-[1,2,4] triazolo [5, 1-f]purin-2-one (51),
5-Amino-8-(2-furyl)-3-[2-[4-(4-methoxyphenyl)-3,6-dihydro-2H-pyridin-1-yl]ethyl]-1-methyl-[1,2,4]triazolo[5,1-f]purin-2-one (52),
5-Amino-8-(2-furyl)-3-[2-[4-[4-(2-methoxy-1,1-dimethyl-ethyl)phenyl]piperazin-1-yl]ethyl]-1-methyl-[1,2,4]triazolo[5,1-f]purin-2-one (53),
5-Amino-8-(2-furyl)-1-methyl-3-(2-piperazin-1-ylethyl)-[1,2,4]triazolo[5,1-f]purin-2-one (54),
5-Amino-8-(2-furyl)-3-[2-[4-(1 H-indole-2-carbonyl)piperazin-1-yl]ethyl]-1-methyl-[1,2,4] triazolo [5, 1-f]purin-2-one (55),
5-Amino-8-(2-furyl)-3-[2-(4-isopropoxyphenyl)ethyl]-1-methyl-[1,2,4] triazolo [5,1-f]purin-2-one (56),
5-Amino-8-(2-furyl)-1-methyl-3-[2-[4-[(2S)-pyrrolidine-2-carbonyl]piperazin-1-yl]ethyl]-[1,2,4]triazolo[5,1-f]purin-2-one (57),
5-Amino-8-(2-furyl)-3-[2-(4-methoxyphenyl)ethyl]-1-methyl-[1,2,4] triazolo [5, 1-f]purin-2-one (58), 5-amino-3-[2-[4-[4-(difluoromethoxy)phenyl]piperazin-1-yl]ethyl]-8-(2-furyl)-1-methyl-[1,2,4] triazolo [5, 1-f]purin-2-one (59),
5-Amino-8-(2-furyl)-1-methyl-3-[2-[4-[3-(5-methyl-1,3 ,4-oxadiazol-2-yl)phenyl]piperazin-1-yl]ethyl]-[1,2,4]triazolo[5,1- f]purin-2-one (60),
5-Amino-3-[2-[4-[2-fluoro-4-(5-methyl-1,2,4-oxadiazol-3-yl)phenyl]piperazin-1-yl]ethyl]-8-(2-furyl)-1-methyl-[1,2,4]triazolo[5,1-f]purin-2-one (61),
5-Amino-3-[2-[4-(6-fluoro-2-methyl-1,3-benzoxazol-5-yl)piperazin-1-yl]ethyl]-8-(2-furyl)-1-methyl-[1,2,4]triazolo[5,1-f]purin-2-one (62),
5-Amino-3-[2-[4-(cyclopropanecarbonyl)piperazin-1-yl]ethyl]-8-(2-furyl)-1-methyl-[1,2,4] triazolo [5, 1-f]purin-2-one (63),
5-Amino-3-[2-[4-(2-cyclopropylacetyl)piperazin-1-yl]ethyl]-8-(2-furyl)-1-methyl-[1,2,4]triazolo[5, 1-f]purin-2-one (64),
5-Amino-8-(2-furyl)-3-[2-[4-[4-(2-hydroxyethoxy)phenyl]piperazin-1-yl]ethyl]-1-methyl-[1,2,4] triazolo [5, 1-f]purin-2-one (65),
5-Amino-8-(2-furyl)-3-[2-[4-(4-hydroxyphenyl)piperazin-1-yl]ethyl]-1-methyl-[1,2,4]triazolo[5,1-f]purin-2-one (66),
5-Amino-1-(cyclopropylmethyl)-3-[2-[4-(4-ethoxyphenyl)piperazin-1-yl]ethyl]-8-(2-furyl)-[1,2,4]triazolo[5,1-f]purin-2-one (67),
5-Amino-1-(cyclopropylmethyl)-3-[2-[4-(4-fluorophenyl)piperazin-1-yl]ethyl]-8-(2-furyl)-[1,2,4]triazolo[5,1-f]purin-2-one (68),
5-Amino-1-(cyclopropylmethyl)-3-[2-[4-(2,4-difluorophenyl)piperazin-1-yl]ethyl]-8-(2-furyl)-[1,2, 4] triazolo [5, 1-f]purin-2-one (69),
5-Amino-1-(cyclopropylmethyl)-8-(2-furyl)-3-[2-[4-[4-(2-methoxyethoxy)phenyl]piperazin-1-yl]ethyl]-[1,2,4]triazolo[5,1-f]purin-2-one (70),
5-Amino-1-(cyclopropylmethyl)-3-[2-(4-fluorophenoxy)ethyl]-8-(2-furyl)-[1,2,4]triazolo[5,1-f]purin-2-one (71),
5-Amino-8-(2-furyl)-1-methyl-3-[2-[2-oxo-5-(trifluoromethyl)-1-pyridyl]ethyl]-[1,2,4]triazolo[5, 1-f]purin-2-one (72),
5-Amino-3-[2-[4-(2,4-difluorophenyl)pyrazol-1-yl]ethyl]-1-ethyl-8-(2-furyl)-[1,2,4]triazolo[5, 1-f]purin-2-one (73),
1-[2-[5-Amino-1-(cyclopropylmethyl)-8-(2-furyl)-2-oxo-[1,2,4]triazolo[5,1-f]purin-3-ylethyl]pyrazole-4-carboxylic acid (74),
1-[2-[5-Amino-8-(2-furyl)-1-methyl-2-oxo-[1,2,4]triazolo[5,1-f]purin-3-yl]ethyl]pyrazole-4-carboxylic acid (75),
1-[2-[5-amino-1-(cyclopropylmethyl)-8-(2-furyl)-2-oxo-[1,2,4]triazolo[5,1-f]purin-3-yl]ethyl]-N-cyclopropyl-pyrazole-4-carboxamide (76),
1-[2-[5-amino-1-(cyclopropylmethyl)-8-(2-furyl)-2-oxo-[1,2,4]triazolo[5,1-f]purin-3-yl]ethyl]-N,N-diethyl-pyrazole-4-carboxamide (77),
1-[2-[5-Amino-1-(cyclopropylmethyl)-8-(2-furyl)-2-oxo-[1,2,4]triazolo[5,1-f]purin-3-yl]ethyl]-N-cyclopropyl-5-methyl-pyrazole-3-carboxamide (78), 2-[2-[5-Amino-1-(cyclopropylmethyl)-8-(2-furyl)-2-oxo-[1,2,4]triazolo[5,1-f]purin-3-yl]ethyl]-N-cyclopropyl-5-methyl-pyrazole-3-carboxamide (79),
1-[2-[5-Amino-8-(2-furyl)-1-methyl-2-oxo-[1,2,4]triazolo[5,1-f]purin-3-yl]ethyl]-N-methyl-pyrazole-3-carboxamide (80),
1-[2-[5-Amino-8-(2-furyl)-1-methyl-2-oxo-[1,2,4]triazolo[5,1-f]purin-3-yl]ethyl]-N,N-diethyl-pyrazole-4-carboxamide (81),
1-[2-[5-amino-8-(2-furyl)-1-methyl-2-oxo-[1,2,4] triazolo [5, 1-f]purin-3-ylethyl]pyrazole-4-carboxamide (82),
5-Amino-8-(2-furyl)-3-[2-[4-[(3R)-3-hydroxypyrrolidine-1-carbonyl]pyrazol-1-yl]ethyl]-1-methyl-[1,2,4]triazolo[5,1-f]purin-2-one (83),
1-[2-[5-Amino-8-(2-furyl)-1-methyl-2-oxo-[1,2,4]triazolo[5,1-f]purin-3-yl]ethyl]-N-methyl-pyrazole-4-carboxamide (84),
1-[2-[5-Amino-8-(2-furyl)-1-methyl-2-oxo-[1,2,4]triazolo[5,1-f]purin-3-yl]ethyl]-N-cycloprop yl-pyrazole-3-carboxamide (85),
1-[2-[5-Amino-8-(2-furyl)-1-methyl-2-oxo-[1,2,4]triazolo[5,1-f]purin-3-yl]ethyl]-N-cyclopropyl-pyrazole-4-carboxamide (86),
5-Amino-8-(2-furyl)-3-[2-[4-(3-hydroxyazetidine-1-carbonyl)pyrazol-1-yl]ethyl]-1-methyl-[1,2,4] triazolo [5, 1-f]purin-2-one (87),
5-Amino-1-ethyl-8-(2-furyl)-3-[2-[4-[4-(2-methoxyethoxy)phenyl]piperazin-1-yl]ethyl]-[1,2,4]triazolo[5,1-f]purin-2-one (88),
5-Amino-3-[2-[4-(2,4-difluorophenyl)piperazin-1-yl]ethyl]-1-ethyl-8-(2-furyl)-[1,2,4]triazolo[5, 1-f]purin-2-one (89),
5-Amino-1-ethyl-3-{2-[4-(4-fluoro-phenyl)-piperidin-1-yl]-ethyl}-8-furan-2-yl-1,3-dihydro-[1,2,4] triazolo [5, 1-f]purin-2-one (90),
5-Amino-1-ethyl-8-(2-furyl)-3-[2-(3-methyl-7,8-dihydro-5H-1,6-naphthyridin-6-yl)ethyl]-[1,2,4]triazolo[5,1-f]purin-2-one (91),
5-Amino-8-(2-furyl)-3-[2-[4-[4-(2-methoxyethoxy)phenyl]piperazin-1-yl]ethyl]-1-(2,2,2-trifluoroethyl)-[1,2,4]triazolo[5,1-f]purin-2-one (92),
5-Amino-3-{2-[4-(2,4-difluoro-phenyl)-piperazin-1-yl]-ethyl}-8-furan-2-yl-1-(2,2,2-trifluoro-ethyl)-1,3-dihydro-[1,2,4]triazolo[5,1-f]purin-2-one (93),
5-Amino-8-(2-furyl)-3-[2-[4-[4-(2-methoxyethoxy)phenyl]piperazin-1-yl]ethyl]-1-(2-methoxyethyl)-[1,2,4]triazolo[5,1-f]purin-2-one (94), 5-amino-3-[2-[4-(4-fluorophenyl)piperazin-1-yl]ethyl]-8-(2-furyl)-1-(2-methoxyethyl)-[1,2,4]triazolo[5,1-f]purin-2-one (95),
5-Amino-3-[2-[4-(4-fluorophenyl)piperazin-1-yl]ethyl]-8-(2-furyl)-1-(2-hydroxyethyl)-[1,2,4]triazolo[5,1-f]purin-2-one one (96),
5-Amino-1-cyclopropyl-8-(2-furyl)-3-[2-[4-[4-(2-methoxyethoxy)phenyl]piperazin-1-yl]ethyl]-[1,2,4]triazolo[5,1-f]purin-2-one (97),
5-Amino-8-(2-furyl)-3-[2-[4-[4-(2-methoxyethoxy)phenyl]piperazin-1-yl]ethyl]-1-(2,2,2-trifluoroethyl)-[1,2,4]triazolo[5,1-f]purin-2-one (98),
5-Amino-3-[2-[4-[4-(2-methoxyethoxy)phenyl]piperazin-1-yl]ethyl]-1-methyl-8-thiazol-2-yl-[1,2,4] triazolo [5, 1-f]purin-2-one (99),
5-Amino-3-[2-[4-[3-fluoro-4-(2-methoxyethoxy)phenyl]piperazin-1-yl]ethyl]-1-methyl-8-thiazol-2-yl-[1,2,4]triazolo[5,1-f]purin-2-one (100),
5-Amino-3-[2-[4-(2-cyclopropylacetyl)piperazin-1-yl]ethyl]-1-methyl-8-thiazol-2-yl-[1,2,4]triazolo[5, 1-f]purin-2-one (101),
5-Amino-3-[2-[4-(4-methoxyphenyl)piperazin-1-yl] ethyl]-1-methyl-8-thiazol-2-yl-[1,2,4]triazolo[5, 1-f]purin-2-one (102),
5-Amino-1-methyl-3-[2-[4-(p-tolyl)piperazin-1-yl]ethyl]-8-thiazol-2-yl-[1,2,4]triazolo[5, 1-f]purin-2-one (103),
5-Amino-1-methyl-3-[2-(3-methyl-7, 8-dihydro-5H-1, 6-naphthyridin-6-yl)ethyl]-8-thiazol-2-yl-[1,2,4] triazolo [5, 1-f]purin-2-one (104),
4-[4-[2-(
5-Amino-1-methyl-2-oxo-8-thiazol-2-yl-[1,2,4]triazolo[5,1-f]purin-3-yl)ethyl] piperazin-1-yl] benzonitrile (105),
5-Amino-1-methyl-3-[2-[4-[3-(5-methyl-1,3 ,4-oxadiazol-2-yl)phenyl]piperazin-1-ylethyl]- 8-thiazol-2-yl-[1,2,4]triazolo[5,1-f]purin-2-one (106),
5-amino-3-[2-[4-[4-(1-hydroxy-1-methyl-ethyl)phenyl]piperazin-1-yl]ethyl]-1-methyl-8-thiazol-2-yl-[1,2,4]triazolo[5,1-f]purin-2-one (107),
5-Amino-3-[2-[4-[4-(2-methoxyethoxy)phenyl]piperazin-1-yl]ethyl]-1-methyl-8-(2-pyridyl)-[1,2,4] triazolo [5, 1-f]purin-2-one (108),
5-Amino-3-[2-[4-(2,4-difluorophenyl)piperazin-1-yl]ethyl]-1-methyl-8-(2-pyridyl)-[1,2,4]triazolo[5, 1-f]purin-2-one (109),
4-[4-[2-[5-Amino-1-methyl-2-oxo-8-(2-pyridyl)-[1,2,4]triazolo[5,1-f]purin-3-yl]ethyl] piperazin-1-yl] benzonitrile (110),
5-Amino-3-[2-[4-[4-(1-hydroxy-1-methyl-ethyl)phenyl]piperazin-1-yl]ethyl]-1-methyl-8-(2-pyridyl)-[1,2,4]triazolo[5,1-f]purin-2-one (111),
5-Amino-3-[2-[4-[4-(2-methoxyethoxy)phenyl]piperazin-1-yl]ethyl]-1-methyl-8-pyrazin-2-yl-[1,2,4] triazolo [5, 1-f]purin-2-one (112),
5-Amino-3-[2-[4-(2,4-difluorophenyl)piperazin-1-yl]ethyl]-1-methyl-8-pyrazin-2-yl-[1,2,4]triazolo[5,1-f]purin-2-one (113),
5-Amino-3-[2-[4-[2-fluoro-4-(2-methoxyethoxy)phenyl]piperazin-1-yl]ethyl]-1-methyl-8-pyrazin-2-yl-[1,2,4]triazolo[5,1-f]purin-2-one (114),
5-Amino-8-(2-furyl)-3-[[1-(4-methoxyphenyl)pyrrolidin-3-yl] methyl]-1-methyl-[1.2.4]triazolo[5,1-f]purin-2-one (115),
5-Amino-8-(2-furyl)-3-[1-[4-(2-methoxyethoxy)phenyl]pyrrolidin-3-yl]methyl]-1-methyl-[1,2,4] triazolo [5, 1-f]purin-2-onehyl}-1-methyl-1,3-dihydro-[1.2.4]triazolo[5,1-i]purin-2-one (116), 5-amino-8-(2-furyl)-3-[2-[4-[4-(2-methoxyethoxy)phenyl]piperazin-1-yl]ethyl]-1-methyl-
[1,2,4] triazolo [5, 1-f]purine-2-thione (117),
8-(2-furyl)-3-[2-[4-[4-(2-methoxyethoxy)phenyl]piperazin-1-yl]ethyl]-1-methyl-5-(methylamino)-[1,2,4]triazolo[5,1-f]purin-2-one (118),
5-Amino-3-{2-[4-(4-fluoro-phenyl)-piperazin-1-yl]-ethyl]-8-isothiazol-5-yl-1-methyl-1,3-dihydro-[1,2,4]triazolo[5,1-i]purin-2-one (119),
5-Amino-8-isothiazol-5-yl-3-(2-{4-[4-(2-methoxy-ethoxy)-phenyl]-piperazin-1-yl]-ethyl)-1-methyl-1,3-dihydro-[1,2,4]triazolo[5,1-i]purin-2-one (120),
5-Amino-3-[2-[4-[2-fluoro-4-(2-methoxyethoxy)phenyl]piperazin-1-yl]ethyl]-8-isothiazol-5-yl-1-methyl-[1,2,4]triazolo[5,1-f]purin-2-one (121),
5-Amino-8-isoxazol-5-yl-3-[2-[4-[4-(2-methoxyethoxy)phenyl]piperazin-1-yl]ethyl]-1-methyl-[1,2,4]triazolo[5,1-f]purin-2-one (122),
5-Amino-3-[2-[4-[4-(2-methoxyethoxy)phenyl]piperazin-1-yl]ethyl]-1-methyl-8-oxazol-2-yl-[1,2,4] triazolo [5, 1-f]purin-2-one (123),
5-Amino-3-{2-[4-(4-methoxy-phenyl)-piperazin-1-yl]-ethyl}-1-methyl-8-prop-1-ynyl-1,3-dihydro-[1,2,4] triazolo [5, 1-i]purin-2-one (124),
5-Amino-3-(2-{4-[4-(2-methoxy-ethoxy)-phenyl]-piperazin-1-yl}-ethyl)-1-methyl-8-prop-l-ynyl-1,3-dihydro-[1,2,4]triazolo[5,1-i]purin-2-one (125),
5-Amino-3-{2-[4-(4-fluoro-benzoyl)-piperazin-1-yl]-ethyl]-8-furan-2-yl-1-methyl-1,3-dihydro-[1,2,4]triazolo[5,1-i]purin-2-one (126),
5-Amino-3-(2-dimethylamino-ethyl)-8-furan-2-yl-1-methyl-1, 3-dihydro-[1,2,4]triazolo[5,1-i]purin-2-one (127),
5-Amino-8-furan-2-yl-3-[3-(4-methoxy-phenyl)-propyl]-1-methyl-1, 3-dihydro-[1,2,4]triazolo[5,1-i]purin-2-one (128),
5-Amino-8-furan-2-yl-1-methyl-3-(2-pyrazol-1-yl-ethyl)-1, 3-dihydro-[1,2,4]triazolo[5,1-i]purin-2-one (129),
5-Amino-8-furan-2-yl-3-(2-{4-[4-(2-methoxy-ethoxy)-phenyl]-pyrazol-1-yl}-ethyl)-1-methyl-1,3-dihydro-[1,2,4]triazolo[5,1-i]purin-2-one (130),
5-Amino-8-furan-2-yl-3-{2-[3-(4-methoxy-phenyl)-pyrrol-1-yl]-ethyl]-1-methyl-1,3-dihydro-[1,2,4]triazolo[5,1-i]purin-2-one (131),
5-Amino-8-furan-2-yl-3-{2-[4-(4-methoxy-phenyl)-imidazol-1-yl]-ethyl}-1-methyl-1,3-dihydro-[1,2,4]triazolo[5,1-i]purin-2-one (132),
5-Amino-8-furan-2-yl-3-{2-[4-(4-methoxy-phenyl)-[1,2,3]triazol-1-yl]-ethyl}-1-methyl-1,3-dihydro-[1,2,4]triazolo[5,1-i]purin-2-one (133),
5-Amino-3-[2-(1,3-dihydro-isoindol-2-yl)-ethyl]-8-furan-2-yl-1-methyl-1,3-dihydro-[1,2,4] triazolo [5, 1-i]purin-2-one (134),
5-Amino-8-furan-2-yl-1-methyl-3-(2-piperidin-1-yl-ethyl)-1, 3-dihydro-[1,2,4]triazolo[5,1-i]purin-2-one (135),
5-Amino-8-furan-2-yl-1-methyl-3-(2-pyrrolidin-1-yl-ethyl)-1,3-dihydro-[1,2,4]triazolo[5,1-i]purin-2-one (136),
5-Amino-8-furan-2-yl-1-methyl-3-[2-(3-methyl-7,8-dihydro-5H-[1,6]naphthyridin- 6-yl)-ethyl]-1,3-dihydro-[1,2,4] triazolo [5, 1-i]purin-2-one (137),
5-Amino-8-furan-2-yl-3-{2-[4-(2-methoxy-ethoxy)-phenoxy]-ethyl}-1-methyl-1,3-dihydro-[1,2,4] triazolo [5, 1-i]purin-2-one (138),
5-Amino-8-furan-2-yl-3-{2-[4-(2-methoxy-ethoxy)-phenylamino]-ethyl]-1-methyl-1,3-dihydro-[1,2,4] triazolo [5, 1-i]purin-2-one (139),
5-Amino-8-furan-2-yl-1-methyl-3-[2-(pyridin-2-yloxy)-ethyl]-1, 3-dihydro-[1,2,4]triazolo[5,1-i]purin-2-one (140),
5-Amino-1-ethyl-3-{2-[4-(4-fluoro-phenyl)-piperazin-1-yl]-ethyl}-8-isothiazol-5-yl-1,3-dihydro-[1,2,4]triazolo[5,1-i]purin-2-one (141),
5-Amino-1-ethyl-8-isothiazol-5-yl-3-(2-{4-[4-(2-methoxy-ethoxy)-phenyl]-piperazin-1-yl}-ethyl)-1,3-dihydro-[1,2,4] triazolo[5, 1-i]purin-2-one (142),
5-Amino-1-ethyl-8-furan-2-yl-3-(2-piperidin-1-yl-ethyl)-1, 3-dihydro-[1,2,4]triazolo[5,1-i]purin-2-one (143),
5-Amino-1-ethyl-8-furan-2-yl-3-[2-(3-methyl-7,8-dihydro-5H-[1,6]naphthyridin-6-yl)-ethyl]-1,3-dihydro-[1,2,4]triazolo[5,1-i]purin-2-one (144),
5-Amino-3-[2-(2,4-difluoro-phenoxy)-ethyl]-1-ethyl-8-furan-2-yl-1,3-dihydro-[1,2,4]triazolo[5,1-i]purin-2-one (145),
5-Amino-3-[2-(2,4-difluoro-phenylamino)-ethyl]-1-ethyl-8-furan-2-yl-1,3-dihydro-[1,2,4]triazolo[5,1-i]purin-2-one (146),
5-Amino-1-cyclopropylmethyl-3-[2-(2,4-difluoro-phenylamino)-ethyl]-8-furan-2-yl-1,3-dihydro-[1,2,4]triazolo[5,1-i]purin-2-one (147),
5-Amino-1-cyclopropylmethyl-3-[2-(2, 4-difluoro-phenoxy)-ethyl]-8-furan-2-yl-1,3-dihydro-[1,2,4] triazolo [5, 1-i]purin-2-one (148),
5-Amino-1-cycloprop ylmethyl-3-{2-[4-(4-fluoro-phenyl)-piperidin-1-yl]-ethyl}-8-furan-2-yl-1,3-dihydro-[1,2,4]triazolo[5,1-i]purin-2-one (149),
5-Amino-3-{2-[4-(4-fluoro-phenyl)-piperidin-1-yl]-ethyl}-8-furan-2-yl-1-(2,2,2-trifluoro-ethyl)-1,3-dihydro-[1,2,4] triazolo[5, 1-i]purin-2-one (150),
5-Amino-8-furan-2-yl-3-{2-[4-(4-methoxy-phenyl)-piperazin-1-yl]-ethyl}-1-(2,2,2-trifluoro-ethyl)-1,3-dihydro-[1,2,4] triazolo[5, 1-i]purin-2-one (151),
5-Amino-3-[2-(4-cyclopropylmethyl-piperazin-1-yl)-ethyl]-8-isothiazol-5-yl-1-(2,2,2-trifluoro-ethyl)-1,3-dihydro-[1,2,4]triazolo[5,1-i]purin-2-one (152),
(5-Amino-8-isothiazol-5-yl-3-{2-[4-(4-methoxy-phenyl)-piperazin-1-yl]-ethyl}-2-oxo-2, 3-dihydro-[1,2, 4]triazolo[5,1-i]purin-1-yl)-acetonitrile (153),
[5-Amino-3-{2-[4-(2,4-difluoro-phenyl)-piperazin-1-yl]-ethyl}-8-(3-fluoro-phenyl)-2-oxo-2,3-dihydro-[1,2,4]triazolo[5,1-i]purin-1-yl]-acetonitrile (154),
[5-Amino-8-furan-2-yl-3-(2-{4-[4-(2-methoxy-ethoxy)-phenyl]-piperazin-1-yl}-ethyl)-2-oxo-2,3-dihydro-[1,2,4]triazolo[5,1-i]purin-1-yl]-acetonitrile (155),
5-Amino-3-(2-{4-[4-(2-methoxy-ethoxy)-phenyl]-piperazin-1-yl}-ethyl)-1-methyl-8-phenyl-1,3-dihydro-[1,2,4]triazolo[5,1-i]purin-2-one (156),
3-[5-Amino-3-(2-{4-[4-(2-methoxy-ethoxy)-phenyl]-piperazin-1-yl}-ethyl)-1-methyl-2-oxo-2, 3-dihydro-1H-[1,2, 4]triazolo[5,1-i]purin-8-yl]-benzonitrile (157),
3-[5-Amino-3-(2-{4-[4-(2-methoxy-ethoxy)-phenyl]-piperazin-1-yl}-ethyl)-1-methyl-2-oxo-2, 3-dihydro-1H-[1,2, 4]triazolo[5,1-i]purin-8-yl]-benzonitrile (158),
5-Amino-8-furan-2-y1-1-methyl-3-vinyl-1,3-dihydro-[1,2,4]triazolo[5,1-i]purin-2-one (159)
5-Amino-3-[3-(4-fluoro-phenyl)-prop-2-ynyl]-8-furan-2-yl-1-methyl-1, 3-dihydro-[1,2,4]triazolo[5,1-i]purin-2-one (160),
5-Amino-8-furan-2-yl-1-methyl-3-[4-(4-methyl-piperazin-1-yl)-but-2-ynyl]-1,3-dihydro-[1,2,4] triazolo [5, 1-i]purin-2-one (161),
5-Amino-8-furan-2-yl-1-isopropyl-3-(2-{4-[4-(2-methoxy-ethoxy)-phenyl]-piperazin-1-yl}-ethyl)-1,3-dihydro-[1,2,4] triazolo[5, 1-i]purin-2-one (162),
5-Amino-2-benzyl-7-(2-{4-[4-(2-methoxy-ethoxy)-phenyl]-piperazin-1-yl}-ethyl)-9-methyl-7,9-dihydro-2H-[1,2,4]triazolo[3,4-i]purine-3,8-dione (163),
5-Amino-2-benzyl-9-methyl-7-(2-morpholin-4-yl-ethyl)-7,9-dihydro-2H-[1,2,4]triazolo[3,4-i]purine-3,8-dione (164),
5-Amino-2-(3-chloro-benzyl)-7-[2-(4-isopropyl-piperazin-1-yl)-ethyl]-9-methyl-7,9-dihydro-2H-[1,2,4]triazolo[3,4-i]purine-3,8-dione (165),
5-Amino-2-cyclopropylmethyl-9-methyl-7-(2-morpholin-4-yl-ethyl)-7,9-dihydro-2H-[1,2,4]triazolo[3,4-i]purine-3,8-dione (166),
5-Amino-2-cyclopropylmethyl-7-(2,4-difluoro-benzyl)-9-methyl-7,9-dihydro-2H-[1,2,4]triazolo[3,4-i]purine-3,8-dione (167),
4-Amino-2-furan-2-yl-6-(2-{4-[4-(2-methoxy-ethoxy)-phenyl]-piperazin-1-yl}-ethyl)-6H-8-oxa-1,3,3a,5,6-pentaaza-as-indacen-7-one (168), and
4-Amino-2-furan-2-yl-6-(2-{4-[4-(2-methoxy-ethoxy)-phenyl]-piperazin-1-yl}-ethyl)-8,8-dimethyl-6, 8-dihydro-1,3, 3a, 5, 6-pentaaza-as-indacen-7-one (169).

In an embodiment of the present disclosure there is provided a pharmaceutical composition comprising compound of Formula II, its pharmaceutically acceptable salts, analogs, tautomeric forms, stereoisomers, geometrical isomers, polymorphs, hydrates, solvates, metabolites, and prodrugs thereof

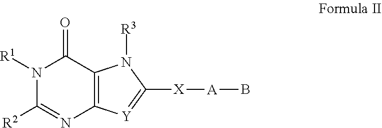

wherein,
Y is selected from N or CR; R is selected from H, hydroxy, alkoxy, alkyl, or aryl; R1 is selected from a group consisting of alkyl, alkenyl and alkynyl, wherein one or more methylene groups are optionally replaced by hetero atoms or groupselected from -0-, —S(0)p-, —N(Ra)—, or —C(O) provided that the heteroatom is not adjacent to N in the ring; p is selected from 0, 1 or 2; wherein alkyl, alkenyl and alkynyl are unsubstituted or substituted independently with alkoxy, acyl, acylamino, acyloxy, amino, monoalkylamino, dialkylamino, aminocarbonyl, alkoxycarbonylamino, azido, cyano, halogen, haloalkyl, hydroxy, hydroxyalkyl, keto, thiocarbonyl, carboxy, alkylcarboxy, carboxyalkyl, —S03 H, aminocarbonylamino, hydroxyamino, alkoxyamino, nitro, —S(0)2 NRa Ra, —NRa S(0)2 Ra, or —S(0)p Ra;
R2 is selected from a group consisting of hydrogen, halogen, cyano, nitro, carboxy, acyl, aminocarbonyl, alkyl, alkenyl, alkynyl, hydroxyalkyl, carboxyalkyl, haloalkyl, haloalkyloxy, alkoxy, —NRb Rb, —S(0)p Rb, cycloalkyl, cycloalkylalkyl, cycloalkyloxy, aryl, arylalkyl, aryloxy, heterocyclyl, heterocyclylalkyl, heterocyclyloxy, heteroaryl, heteroarylalkyl and heteroaryloxy; wherein alkyl, alkenyl, alkynyl, alkoxy, carboxyalkyl, cycloalkyl, cycloalkylalkyl, cycloalkyloxy, aryl, arylalkyl, aryloxy, heterocyclyl, heterocyclylalkyl, heterocyclyloxy, heteroaryl, heteroarylalkyl, heteroaryloxy and Rb are unsubstituted or substituted independently with alkyl, alkenyl, alkynyl, alkoxy, acyl, acylamino, acyloxy, nitro, amino, monoalkylamino, dialkylamino, hydroxyamino, alkoxyamino, aminocarbonylamino, azido, cyano, halogen, hydroxy, hydroxyalkyl, keto, thiocarbonyl, carboxy, alkylcarboxy, carboxyalkyl, —SO3H, arylamino, cycloalkylamino, heteroarylamino, heterocyclylamino, aminocarbonyl, alkoxycarbonylamino, cycloalkyl, cycloalkyloxy, cycloalkenyl, aryl, aryloxy, heteroaryl, heteroaryloxy, heterocyclyl, heterocyclyloxy, —S(0)2 NRc Rc, —NRC S(0)2 RC or —S(0)p Rd; wherein each substituent is unsubstituted or substituted with 1, 2, or 3 substituents independently selected from alkyl, carboxy, carboxyalkyl, aminocarbonyl, hydroxy, alkoxy, halogen, haloalkyl, haloalkoxy, amino, substituted amino, cyano or —S(0)p Rd;
R3 is selected from a group consisting of hydrogen, alkyl, alkenyl, alkynyl, alkoxyalkyl, cycloalkyl, cycloalkylalkyl, aryl, arylalkyl, heteroaryl and heteroarylalkyl; wherein alkyl, alkenyl, alkynyl, alkoxyalkyl, cycloalkyl, cycloalkylalkyl, aryl, arylalkyl, heteroaryl, and heteroarylalkyl are unsubstituted or substituted independently with alkyl, alkenyl, alkynyl, alkoxy, cycloalkyl, cycloalkenyl, acyl, acylamino, acyloxy, amino, monoalkylamino, dialkylamino, arylamino, cycloalkylamino, heteroarylamino, heterocyclylamino, aminocarbonyl, alkoxycarbonylamino, azido, cyano, halogen, hydroxy, hydroxyalkyl, keto, thiocarbonyl, carboxy, alkylcarboxy, carboxyalkyl, carboxyalkyloxy, alkylcarboxyalkyloxy, —S03 H, aryl, aryloxy, cycloalkyloxy, heteroaryl, aminocarbonylamino, heteroaryloxy, heterocyclyl, heterocyclyloxy, hydroxyamino, alkoxyamino, nitro, S(0)2 NRc Rc, —NRc S(0)2 Rc or —S(0)p Rd; wherein cach substituent is unsubstituted or substituted with 1, 2, or 3 substituents independently selected from alkyl, carboxy, carboxyalkyl, aminocarbonyl, hydroxyd, alkoxy, halogen, haloalkyl, haloalkoxy, amino, substituted amino, cyano or —S(0)p Rd;
X is either an optionally substituted arylene or an optionally substituted heteroarylene;
A is selected from a bond, (Ci-C6)alkylene, (C2—C6)alkenylene or (C2-Cejalkynylenc group, wherein 1 to 4 methylene groups are optionally replaced by groups independently selected from O, —S(0)p-, —N(Rb)—, or —C(O)—; wherein alkylene, alkenylene, and alkynylene are unsubstituted or substituted independently with alkyl, alkenyl, alkynyl, alkoxy, cycloalkyl, cycloalkenyl, acyl, acylamino, acyloxy, amino, monoalkylamino, dialkylamino, arylamino, cycloalkylamino, heteroarylamino, heterocyclylamino, aminocarbonyl, alkoxycarbonylamino, azido, cyano, halogen, hydroxy, hydroxyalkyl, keto, thiocarbonyl, carboxy, alkylcarboxy, carboxyalkyl, carboxyalkyloxy, alkylcarboxyalkyloxy—SO3H, aryl, aryloxy, cycloalkyloxy, heteroaryl, aminocarbonylamino, heteroaryloxy, heterocyclyl, heterocyclyloxy, hydroxyamino, alkoxyamino, nitro, S(0)2 NRc Rc, —NRc S(0)2 Rc or —S(0)p Rd; wherein each substituent is unsubstituted or substituted with 1, 2, or 3 substituents independently selected from alkyl, carboxy, carboxyalkyl, aminocarbonyl, hydroxy, alkoxy, halogen, CF3, amino, substituted amino, cyano or —S(0)p Rd;
B is selected from hydrogen, heterocyclyl, cycloalkyl, aryl or heteroaryl; wherein heterocyclyl, cycloalkyl, aryl and heteroaryl are unsubstituted or substituted independently with alkyl, alkenyl, alkynyl, alkoxy, cycloalkyl, cycloalkylalkyl, cycloalkenyl, acyl, acylamino, acyloxy, amino, monoalkylamino, dialkylamino, arylamino, cycloalkylamino, heteroarylamino, heterocyclylamino, aminocarbonyl, alkoxycarbonylamino, azido, cyano, halogen, hydroxy, hydroxyalkyl, keto, thiocarbonyl, carboxy, alkylcarboxy, carboxyalkyl, carboxyalkyloxy, alky Icarboxy alky loxy —S03 H, aryl, arylalkyl, aryloxy, cycloalkyloxy, heteroaryl, heteroarylalkyl, aminocarbonylamino, heteroaryloxy, heterocyclyl, heterocyclylalkyl, heterocyclyloxy, hydroxyamino, alkoxyamino, nitro, S(0)2 NRb Rb, —NRb S(0)2 Rb or —S(0)p Rd; wherein cach substituent is unsubstituted or substituted with 1, 2, or 3 substituents independently selected from alkyl, carboxy, carboxyalkyl, aminocarbonyl, hydroxy, alkoxy, alkoxyalkoxy, alkoxyalkyl, halogen, haloalkyl, haloalkoxy, amino, substituted amino, cyano or —S(0)P Rd;
Ra is independently selected from hydrogen or alkyl;
Rb is independently selected from the group consisting of hydrogen, alkyl, acyl, carboxyalkyl, carbonylamino, cycloalkyl, cycloalkylalkyl, aryl, arylalkyl, heteroaryl, heteroarylalkyl, heterocyclyl, and heterocyclylalkyl;
Rc is selected from hydrogen, alkyl, aryl, heteroaryl or heterocyclyl;
Rd is selected from alkyl, cycloalkyl, aryl, heterocyclyl or heteroaryl; and p is 0, 1 or 2, for the manufacture of a medicament for the treatment of a condition or disorder ameliorated by inhibition of the A2A/A2B receptor.

In an embodiment of the present disclosure, there is provided a pharmaceutical composition comprising compound of Formula II, its pharmaceutically acceptable salts, analogs, tautomeric forms, stercoisomers, geometrical isomers, polymorphs, hydrates, solvates, metabolites, and prodrugs thereof, wherein
Y is N;
R1 is selected from a group consisting of alkyl, alkenyl and alkynyl, wherein alkyl, alkonyl and alkynyl are unsubstituted or substituted independently with alkoxy, acyl, acylamino, acyloxy, amino, monoalkylamino, dialkylamino, aminocarbonyl, alkoxycarbonylamino, azido, cyano, halogen, haloalkyl, hydroxyl, hydroxyalkyl, kcto, thiocarbonyl, carboxy, alkylcarboxy or carboxyalkyl; R2 is selected from a group consisting of hydrogen, halogen, cyano, nitro, carboxy, acyl, aminocarbonyl, alkyl, alkenyl, alkynyl, hydroxyalkyl, carboxyalkyl, haloalkyl, haloalkyloxy, alkoxy, —NRb Rb, —S(0)p Rb, cycloalkyl, cycloalkylalkyl, cycloalkyloxy, aryl, arylalkyl, aryloxy, heterocyclyl, heterocyclylalkyl, heterocyclyloxy, heteroaryl, heteroarylalkyl and heteroaryloxy;
wherein alkyl, alkenyl, alkynyl, alkoxy, carboxyalkyl, cycloalkyl, cycloalkylalkyl, cycloalkyloxy, aryl, arylalkyl, aryloxy, heterocyclyl, heterocyclylalkyl, heterocyclyloxy, heteroaryl, heteroarylalkyl, heteroaryloxy and Rb are unsubstituted or substituted independently with alkyl, alkenyl, alkynyl, alkoxy, acyl, acylamino, acyloxy, nitro, amino, monoalkylamino, dialkylamino, hydroxyamino, alkoxyamino, aminocarbonylamino, azido, cyano, halogen, hydroxyl, hydroxyalkyl, kcto, thiocarbonyl, carboxy, alkylcarboxy, carboxyalkyl, —S03 H, arylamino, cycloalkylamino, heteroarylamino, heterocyclylamino, aminocarbonyl, alkoxycarbonylamino, cycloalkyl, cycloalkyloxy, cycloalkenyl, aryl, aryloxy, heteroaryl, heteroaryloxy, heterocyclyl, heterocyclyloxy, —S(0)2 NRc Rc, —NRC S(0)2 RC or —S(0)pRd;
wherein cach substituent is unsubstituted or substituted with 1, 2, or 3 substituents independently selected from alkyl, carboxy, carboxyalkyl, aminocarbonyl, hydroxyl, alkoxy, halogen, haloalkyl, haloalkoxy, amino, substituted amino, cyano or —S(0)pRd;
R3 is selected from a group consisting of hydrogen, alkyl, alkenyl, alkynyl, alkoxyalkyl, cycloalkyl, cycloalkylalkyl, aryl, arylalkyl, heteroaryl and heteroarylalkyl;
wherein alkyl, alkenyl, alkynyl, alkoxyalkyl, cycloalkyl, cycloalkylalkyl, aryl, arylalkyl, heteroaryl, and heteroarylalkyl are unsubstituted or substituted independently with alkyl, alkenyl, alkynyl, alkoxy, cycloalkyl, cycloalkenyl, acyl, acylamino, acyloxy, amino, monoalkylamino, dialkylamino, arylamino, cycloalkylamino, heteroarylamino, heterocyclylamino, aminocarbonyl, alkoxycarbonylamino, azido, cyano, halogen, hydroxyl, hydroxyalkyl, keto, thiocarbonyl, carboxy, alkylcarboxy, carboxyalkyl, carboxyalkyloxy, alkylcarboxyalkyloxy, —SO3H, aryl, aryloxy, cycloalkyloxy, heteroaryl, aminocarbonylamino, heteroaryloxy, heterocyclyl, heterocyclyloxy, hydroxyamino, alkoxyamino, nitro, S(0)2 NRc Rc, —NRc S(0)2 Rc or —S(0)p Rd;
wherein cach substituent is unsubstituted or substituted with 1, 2, or 3 substituents independently selected from alkyl, carboxy, carboxyalkyl, aminocarbonyl, hydroxyd, alkoxy, halogen, CF3, amino, substituted amino, cyano or —S(0)p Rd;
X is either an optionally substituted arylene or an optionally substituted heteroarylene;
A is selected from a bond, (Ci-C6)alkylene, (C2—C6)alkenylene or (C2—Cejalkynylenc group, wherein 1 to 4 methylene groups are optionally replaced by groups independently selected from 0, —S(0)p-, —N(Rb)—, or —C(O)—;
wherein alkylene, alkenylene, and alkynylene are unsubstituted or substituted independently with alkyl, alkenyl, alkynyl, alkoxy, cycloalkyl, cycloalkenyl, acyl, acylamino, acyloxy, amino, monoalkylamino, dialkylamino, arylamino, cycloalkylamino, heteroarylamino, heterocyclylamino, aminocarbonyl, alkoxycarbonylamino, azido, cyano, halogen, hydroxyl, hydroxyalkyl, kcto, thiocarbonyl, carboxy, alkylcarboxy, carboxyalkyl, carboxyalkyloxy, alky lcarboxy alky loxy —S03 H, aryl, aryloxy, cycloalkyloxy, heteroaryl, aminocarbonylamino, heteroaryloxy, heterocyclyl, heterocyclyloxy, hydroxyamino, alkoxyamino, nitro, S(0)2 NRc Rc, —NRc S(0)2 Rc or —S(0)p Rd; wherein cach substituent is unsubstituted or substituted with 1, 2, or 3 substituents independently selected from alkyl, carboxy, carboxyalkyl, aminocarbonyl, hydroxyl, alkoxy, halogen, CF3, amino, substituted amino, cyano or —S(0)p Rd;
B is selected from hydrogen, heterocyclyl, cycloalkyl, aryl or heteroaryl;
wherein heterocyclyl, cycloalkyl, aryl and heteroaryl are unsubstituted or substituted independently with alkyl, alkenyl, alkynyl, alkoxy, cycloalkyl, cycloalkylalkyl, cycloalkenyl, acyl, acylamino, acyloxy, amino, monoalkylamino, dialkylamino, arylamino, cycloalkylamino, heteroarylamino, heterocyclylamino, aminocarbonyl, alkoxycarbonylamino, azido, cyano, halogen, hydroxyl, hydroxyalkyl, kcto, thiocarbonyl, carboxy, alkylcarboxy, carboxyalkyl, carboxyalkyloxy, alkylcarboxyalkyloxy —S03 H, aryl, arylalkyl, aryloxy, cycloalkyloxy, heteroaryl, heteroarylalkyl, aminocarbonylamino, heteroaryloxy, heterocyclyl, heterocyclylalkyl, heterocyclyloxy, hydroxyamino, alkoxyamino, nitro, S(0)2 NRb Rb, —NRb S(0)2 Rb or —S(0)P Rd;
wherein cach substituent is unsubstituted or substituted with 1, 2, or 3 substituents independently selected from alkyl, carboxy, carboxyalkyl, aminocarbonyl, hydroxyl, alkoxy, alkoxyalkoxy, alkoxyalkyl, halogen, haloalkyl, haloalkoxy, amino, substituted amino, cyano or —S(0)p Rd;
Ra is independently selected from the group consisting of hydrogen and alkyl;
Rb is independently selected from the group consisting of hydrogen, alkyl, acyl, carboxyalkyl, carbonylamino, cycloalkyl, cycloalkylalkyl, aryl, arylalkyl, heteroaryl heteroarylalkyl, heterocyclyl and heterocyclylalkyl;
Rc is selected from hydrogen, alkyl, aryl, heteroaryl or heterocyclyl;
Rd is selected from alkyl, cycloalkyl, aryl, heterocyclyl or heteroaryl;
and p is 0, 1 or 2, for the manufacture of a medicament for the treatment of a condition or disorder ameliorated by inhibition of the A2A/A2B receptor.

In an embodiment of the present disclosure, there is provided a pharmaceutical composition comprising compound of Formula II, its pharmaceutically acceptable salts, analogs, tautomeric forms, stereoisomers, geometrical isomers, polymorphs, hydrates, solvates, metabolites, and prodrugs thereof, wherein Y is CR; R is selected from the group consisting of H, hydroxy, alkoxy, alkyl, and aryl;
R1 is selected from the group consisting of alkyl, alkenyl and alkynyl, wherein alkyl, alkenyl and alkynyl are unsubstituted or substituted independently with alkoxy, acyl, acylamino, acyloxy, amino, monoalkylamino, dialkylamino, aminocarbonyl, alkoxycarbonylamino, azido, cyano, halogen, haloalkyl, hydroxy, hydroxyalkyl, keto, thiocarbonyl, carboxy, alkylcarboxy, or carboxyalkyl;
R2 is selected from the group consisting of hydrogen, halogen, cyano, nitro, carboxy, acyl, aminocarbonyl, alkyl, alkenyl, alkynyl, hydroxyalkyl, haloalkyl, haloalkyloxy, alkoxy, and—NRb Rb;
wherein alkyl, alkenyl, alkynyl, alkoxy and Rb are unsubstituted or substituted independently with alkyl, alkenyl, alkynyl, alkoxy, acyl, acylamino, acyloxy, nitro, amino, monoalkylamino, dialkylamino, hydroxyamino, alkoxyamino, aminocarbonylamino, azido, cyano, halogen, hydroxy, hydroxyalkyl, kcto, thiocarbonyl, carboxy, alkylcarboxy, carboxyalkyl, —S03 H, arylamino, cycloalkylamino, heteroarylamino, heterocyclylamino, aminocarbonyl, alkoxycarbonylamino, cycloalkyl or cycloalkenyl;
R3 is selected from the group consisting of hydrogen, alkyl, alkenyl, alkynyl, cycloalkyl, cycloalkylalkyl, aryl, arylalkyl, heteroaryl and heteroarylalkyl;
X is optionally substituted heteroarylene;
A is selected from the group consisting of a bond, (Ci-C6)alkylene, (C2-Cejalkenylenc and (C2—C6)alkynylene group, wherein 1 to 4 methylene groups are optionally replaced by groups independently selected from the group consisting of 0, —S(0)P, —N(Rb)—, and —C(O)—;
B is selected from the group consisting of heterocyclyl, cycloalkyl, aryl and heteroaryl;
wherein heterocyclyl, cycloalkyl, aryl and heteroaryl are unsubstituted or substituted independently with alkyl, alkenyl, alkynyl, alkoxy, cycloalkyl, cycloalkylalkyl, cycloalkenyl, acyl, acylamino, acyloxy, amino, monoalkylamino, dialkylamino, arylamino, cycloalkylamino, heteroarylamino, heterocyclylamino, aminocarbonyl, alkoxycarbonylamino, azido, cyano, halogen, hydroxy, hydroxyalkyl, keto, thiocarbonyl, carboxy, alkylcarboxy, carboxyalkyl, carboxyalkyloxy, alkylcarboxyalkyloxy —SO3 H, aryl, arylalkyl, aryloxy, cycloalkyloxy, heteroaryl, heteroarylalkyl, aminocarbonylamino, heteroaryloxy, heterocyclyl, heterocyclylalkyl, heterocyclyloxy, hydroxyamino, alkoxyamino, nitro, S(0)2 NRb Rb, —NRb S(0)2 Rb or —S(0)P Rd;
wherein each substituent is unsubstituted or substituted with 1, 2, or 3 substituents independently selected from the group consisting of alkyl, carboxy, carboxyalkyl, aminocarbonyl, hydroxy, alkoxy, alkoxyalkoxy, alkoxyalkyl, halogen, haloalkyl, haloalkoxy, amino, substituted amino, cyano and —S(0)p Rd;
Rb is independently selected from the group consisting of hydrogen, alkyl, acyl, carboxyalkyl, carbonylamino, cycloalkyl, cycloalkylalkyl, aryl, arylalkyl, heteroaryl heteroarylalkyl, heterocyclyl and heterocyclylalkyl; Rd is selected from the group consisting of alkyl, cycloalkyl, aryl, heterocyclyl and heteroaryl;
and p is 0, 1 or 2, for the manufacture of a medicament for the treatment of a condition or disorder ameliorated by inhibition of the A2A/A2B receptor.

In an embodiment of the present disclosure, there is provided a pharmaceutical composition comprising compound of Formula II, its pharmaceutically acceptable salts, analogs, tautomeric forms, stereoisomers, geometrical isomers, polymorphs, hydrates, solvates, metabolites, and prodrugs thereof, wherein Y is N or CR; R is selected from the group consisting of H, hydroxy, alkoxy, alkyl, and aryl;
R1 is selected from the group consisting of alkyl, alkenyl and alkynyl;
R2 is selected from the group consisting of heterocyclyl, heterocyclyloxy, heteroaryl and heteroaryloxy;
wherein heterocyclyl, heterocyclyloxy, heteroaryl, and heteroaryloxy are unsubstituted or substituted independently with alkyl, alkenyl, alkynyl, alkoxy, acyl, acylamino, acyloxy, nitro, amino, monoalkylamino, dialkylamino, hydroxyamino, alkoxyamino, aminocarbonylamino, azido, cyano, halogen, hydroxy, hydroxyalkyl, keto, thiocarbonyl, carboxy, alkylcarboxy, carboxyalkyl, —S03 H, arylamino, cycloalkylamino, heteroarylamino, heterocyclylamino, aminocarbonyl, alkoxycarbonylamino, cycloalkyl or cycloalkenyl;
R3 is selected from the group consisting of hydrogen, alkyl, alkenyl, alkynyl, alkoxyalkyl, cycloalkyl, cycloalkylalkyl, aryl, arylalkyl, heteroaryl and heteroarylalkyl;
X is an optionally substituted phenyl;
A is selected from the group consisting of a bond, (Ci-C6)alkylene, (C2-Cejalkenylenc and (C2—C6)alkynylene group, wherein 1 to 4 methylene groups are optionally replaced by groups independently selected from the group consisting of 0, —S(0)p-, —N(Rb)—, and —C(O)—,
B is selected from the group consisting of heterocyclyl, cycloalkyl, aryl and heteroaryl; wherein heterocyclyl, cycloalkyl, aryl and heteroaryl are unsubstituted or substituted independently with alkyl, alkenyl, alkynyl, alkoxy, cycloalkyl, cycloalkylalkyl, cycloalkenyl, acyl, acylamino, acyloxy, amino, monoalkylamino, dialkylamino, arylamino, cycloalkylamino, heteroarylamino, heterocyclylamino, aminocarbonyl, alkoxycarbonylamino, azido, cyano, halogen, hydroxy, hydroxyalkyl, keto, thiocarbonyl, carboxy, alkylcarboxy, carboxyalkyl, carboxyalkyloxy, alkylcarboxyalkyloxy —S03 H, aryl, arylalkyl, aryloxy, cycloalkyloxy, heteroaryl, heteroarylalkyl, aminocarbonylamino, heteroaryloxy, heterocyclyl, heterocyclylalkyl, heterocyclyloxy, hydroxyamino, alkoxyamino, nitro, S(0)2 NRb Rb, —NRb S(0)2 Rb or —S(0)P Rd;
wherein cach substituent is unsubstituted or substituted with 1, 2, or 3 substituents independently selected from the group consisting of alkyl, carboxy, carboxyalkyl, aminocarbonyl, hydroxy, alkoxy, alkoxyalkoxy, alkoxyalkyl, halogen, haloalkyl, haloalkoxy, amino, substituted amino, cyano and —S(0)p Rd;
Rb is independently selected from the group consisting of hydrogen, alkyl, acyl, carboxyalkyl, carbonylamino, cycloalkyl, cycloalkylalkyl, aryl, arylalkyl, heteroaryl hetcroarylalkyl, heterocyclyl and heterocyclylalkyl;
Rd is selected from the group consisting of alkyl, cycloalkyl, aryl, heterocyclyl and heteroaryl;
and p is 0, 1 or 2, for the manufacture of a medicament for the treatment of a condition or disorder ameliorated by inhibition of the A2A/A2B receptor.

In an embodiment of the present disclosure, there is provided a pharmaceutical composition comprising compound of Formula II, its pharmaceutically acceptable salts, analogs, tautomeric forms, stereoisomers, geometrical isomers, polymorphs, hydrates, solvates, metabolites, and prodrugs thereof, wherein Y is N;
R1 is an alkyl, wherein one or more methylene groups are replaced by hetero atoms or groups such as —O—, —S(0)p-, —N(Ra)—, or —C(O) provided that the heteroatom is not adjacent to N in the ring; p is 0, 1 or 2;
wherein alkyl is unsubstituted or substituted independently with alkoxy, acyl, acylamino, acyloxy, amino, monoalkylamino, dialkylamino, aminocarbonyl, alkoxycarbonylamino, cyano, halogen, haloalkyl, hydroxy, hydroxyalkyl, thiocarbonyl, carboxy, alkylcarboxy, carboxyalkyl, —S03 H, aminocarbonylamino, hydroxyamino, alkoxyamino, —S(0)2 NRa Ra, —NRa S(0)2 Ra, or —S(0)p Ra;
R2 is selected from the group consisting heterocyclyl, heterocyclylalkyl, heterocyclyloxy, heteroaryl, heteroarylalkyl and heteroaryloxy;
wherein heterocyclyl, heterocyclylalkyl, heterocyclyloxy, heteroaryl, heteroarylalkyl, and heteroaryloxy are unsubstituted or substituted independently with alkyl, alkenyl, alkynyl, alkoxy, acyl, acylamino, acyloxy, nitro, amino, monoalkylamino, dialkylamino, hydroxyamino, alkoxyamino, aminocarbonylamino, azido, cyano, halogen, hydroxy, hydroxyalkyl, keto, thiocarbonyl, carboxy, alkylcarboxy, carboxyalkyl, —SO3H, arylamino, cycloalkylamino, heteroarylamino, heterocyclylamino, aminocarbonyl, alkoxycarbonylamino, cycloalkyl, cycloalkyloxy, cycloalkenyl, aryl, aryloxy, heteroaryl, heteroaryloxy, heterocyclyl, heterocyclyloxy, —S(0)2 NRc Rc, —NRC S(0)2 RC or —S(0)pRd;
wherein cach substituent is unsubstituted or substituted with 1, 2, or 3 substituents independently selected from the group consisting of alkyl, carboxy, carboxyalkyl, aminocarbonyl, hydroxy, alkoxy, halogen, haloalkyl, haloalkoxy, amino, substituted amino, cyano and —S(0)p Rd;
R3 is selected from the group consisting of hydrogen, alkyl and arylalkyl;
X is an optionally substituted heteroarylene;
A is selected from the group consisting of (Ci-C6)alkylene, (C2—C6)alkenylene and (C2—C6) alkynylene group, wherein 1 to 4 methylene groups are optionally replaced by groups independently selected from the groups consisting of 0, —S(0)p-, —N(Rb)—, and —C(O)—;
wherein alkylene, alkenylene, and alkynylene are unsubstituted or substituted independently with alkyl, alkoxy, cycloalkyl, halogen, hydroxy, hydroxyalkyl, carboxy, alkylcarboxy, carboxyalkyl, carboxyalkyloxy, —SO3H, hydroxyamino, alkoxyamino, S(0)2 NRc Rc, —NRc S(0)2 Rc or —S(0)P Rd;
B is selected from the group consisting of heterocyclyl, cycloalkyl, aryl and heteroaryl; wherein heterocyclyl, cycloalkyl, aryl and heteroaryl are unsubstituted or substituted independently with alkyl, alkenyl, alkynyl, alkoxy, cycloalkyl, cycloalkylalkyl, cycloalkenyl, acyl, acylamino, acyloxy, amino, monoalkylamino, dialkylamino, arylamino, cycloalkylamino, heteroarylamino, heterocyclylamino, aminocarbonyl, alkoxycarbonylamino, azido, cyano, halogen, hydroxy, hydroxyalkyl, kcto, thiocarbonyl, carboxy, alkylcarboxy, carboxyalkyl, carboxyalkyloxy, alkylcarboxyalkyloxy —S03 H, aryl, arylalkyl, aryloxy, cycloalkyloxy, heteroaryl, heteroarylalkyl, aminocarbonylamino, heteroaryloxy, heterocyclyl, heterocyclylalkyl, heterocyclyloxy, hydroxyamino, alkoxyamino, nitro, —S(0)2 NRb Rb, —NRb S(0)2 Rb or —S(0)P Rd;
wherein each substituent is unsubstituted or substituted with 1, 2, or 3 substituents independently selected from the group consisting of alkyl, carboxy, carboxyalkyl, aminocarbonyl, hydroxy, alkoxy, alkoxyalkoxy, alkoxyalkyl, halogen, haloalkyl, haloalkoxy, amino, substituted amino, cyano and —S(0)p Rd;
Ra is independently selected from the group consisting of hydrogen and alkyl;
Rb is independently selected from the group consisting of hydrogen, alkyl, acyl, carboxyalkyl, carbonylamino, cycloalkyl, cycloalkylalkyl, aryl, arylalkyl, heteroaryl heteroarylalkyl, heterocyclyl and heterocyclylalkyl;
Rc is selected from the group consisting of hydrogen, alkyl, aryl, heteroaryl and heterocyclyl;
Rd is selected from the group consisting of alkyl, cycloalkyl, aryl, heterocyclyl and heteroaryl;
and p is 0, 1 or 2, for the manufacture of a medicament for the treatment of a condition or disorder ameliorated by inhibition of the A2A/A2B receptor.

In an embodiment of the present disclosure, there is provided a pharmaceutical composition comprising compound of Formula II, its pharmaceutically acceptable salts, analogs, tautomeric forms, stereoisomers, geometrical isomers, polymorphs, hydrates, solvates, metabolites, and prodrugs thereof, for use in the treatment of a condition or disorder selected from prostate cancer, rectal cancer, renal cancer, ovarian cancer, endometrial cancer, thyroid cancer, pancreatic cancer, breast cancer, colon cancer, bladder cancer, brain cancer, glial cancer, melanoma cancer, pineal gland cancer, or lung cancer.

In an embodiment of the present disclosure, there is provided a method of using the pharmaceutical composition comprising compound of Formula II, its pharmaceutically acceptable salts, analogs, tautomeric forms, stereoisomers, geometrical isomers, polymorphs, hydrates, solvates, metabolites, and prodrugs thereof, in the treatment of a disease or condition in a mammal that is amenable to treatment with an A2 A/A2B receptor antagonist, the method comprising: administering to a mammal in need thereof a therapeutically effective dose of the pharmaceutical composition as disclosed herein.

In an embodiment of the present disclosure, there is provided a method of treatment of a disorder or condition ameliorated by antagonizing the A2 A/A2B receptor, the method comprising: administering an effective amount of the pharmaceutical composition comprising compound of Formula II, its pharmaceutically acceptable salts, analogs, tautomeric forms, stereoisomers, geometrical isomers, polymorphs, hydrates, solvates, metabolites, and prodrugs thereof, to a patient in need of such treatment.

In an embodiment of the present disclosure, there is provided a use of the pharmaceutical composition comprising compound of Formula II, its pharmaceutically acceptable salts, analogs, tautomeric forms, stereoisomers, geometrical isomers, polymorphs, hydrates, solvates, metabolites, and prodrugs thereof, for the preparation of a medicament for the treatment of a condition or disorder selected from prostate cancer, rectal cancer, renal cancer, ovarian cancer, endometrial cancer, thyroid cancer, pancreatic cancer, breast cancer, colon cancer, bladder cancer, brain cancer, glial cancer, melanoma cancer, pineal gland cancer, or lung cancer.

In an embodiment of the present disclosure, there is provided a pharmaceutical composition comprising compound of Formula II, its pharmaceutically acceptable salts, analogs, tautomeric forms, stereoisomers, geometrical isomers, polymorphs, hydrates, solvates, metabolites, and prodrugs thereof, in combination with at least one PD-LI antibody for use in the treatment of a condition or disorder selected from prostate cancer, rectal cancer, renal cancer, ovarian cancer, endometrial cancer, thyroid cancer, pancreatic cancer, breast cancer, colon cancer, bladder cancer, brain cancer, glial cancer, melanoma cancer, pineal gland cancer, or lung cancer.

In an embodiment of the present disclosure, there is provided a use of the pharmaceutical composition comprising compound of Formula II, its pharmaceutically acceptable salts, analogs, tautomeric forms, stereoisomers, geometrical isomers, polymorphs, hydrates, solvates, metabolites, and prodrugs thereof, in combination with at least one PD-LI antibody for use in the treatment of a condition or disorder selected from prostate cancer, rectal cancer, renal cancer, ovarian cancer, endometrial cancer, thyroid cancer, pancreatic cancer, breast cancer, colon cancer, bladder cancer, brain cancer, glial cancer, melanoma cancer, pineal gland cancer, or lung cancer.

In an embodiment of the present disclosure, there is provided a pharmaceutical composition comprising compound of Formula II, its pharmaceutically acceptable salts, analogs, tautomeric forms, stereoisomers, geometrical isomers, polymorphs, hydrates, solvates, metabolites, and prodrugs thereof, wherein the compound of Formula II is selected from the group consisting of:
8-(4-Benzyloxy-phenyl)-1-propyl-1,7-dihydro-purin-6-one (170),
1-Propyl-8-[1-(3-trifluoromethyl-benzyl)-1 H-pyrazol-4-yl]-1,7-dihydro-purin-6-one (171),
8-(1-Benzyl-1H-pyrazol-4-yl)-1-propyl-1,7-dihydro-purin-6-one (170),
2-Chloro-8-[1-(2,3-difluoro-benzyl)-1H-pyrazol-4-yl]-1-propyl-1,7-dihydro-purin-6-one (172),
2-Chloro-8-[1-(3-fluoro-4-trifluoromethyl-benzyl)-1H-pyrazol-4-yl]-1-propyl-1,7-dihydro-purin-6-one (173),
2-Chloro-1-propyl-8-[1-(4-trifluoromethyl-benzyl)-1H-pyrazol-4-yl]-1, 7-dihydro-purin-6-one (174),
8-[1-(3-Fluoro-4-trifluoromethyl-benzyl)-1H-pyrazol-4-yl]-1-propyl-1, 7-dihydro-purin-6-one (175), 8-[1-(2,3-Difluoro-benzyl)-1H-pyrazol-4-yl]-1-propyl-1,7-dihydro-purin-6-one (176),
1-Propyl-8-[1-(4-trifluoromethyl-benzyl)-1 H-pyrazol-4-yl]-1,7-dihydro-purin-6-one (177),
1-Propyl-8-(1H-pyrazol-4-yl)-1,7-dihydro-purin-6-one (178),
2-Chloro-8-[1-(3-fluoro-benzyl)-1H-pyrazol-4-yl]-1-propyl-1,7-dihydro-purin-6-one (179),
8-[1-(2,4-Difluoro-benzyl)-1H-pyrazol-4-yl]-1-propyl-1,7-dihydro-purin-6-one (180),
2-Chloro-1-propyl-8-[1-(3-trifluoromethyl-benzyl)-1H-pyrazol-4-yl]-1, 7-dihydro-purin-6-one (181),
8-{4-[3-(4-Fluoro-phenyl)-prop-2-ynyloxy]-phenyl}-1-propyl-1,7-dihydro-purin-6-one (182),
8-{4-[5-Oxo-1-(4-trifluoromethoxy-phenyl)-pyrrolidin-3-ylmethoxy]-phenyl}-1-propyl-1,7-dihydro-purin-6-one (183),
1-Propyl-8-{4-[3-(3-trifluoromethyl-phenyl)-prop-2-ynyloxy]-phenyl}-1,7-dihydro-purin-6-one (184),
8-{4-[5-Oxo-1-(3-trifluoromethyl-phenyl)-pyrrolidin-3-ylmethoxy]-phenyl}-1-propyl-1,7-dihydro-purin-6-one (185),
2-Chloro-8-[1-(2,4-difluoro-benzyl)-1H-pyrazol-4-yl]-1-propyl-1,7-dihydro-purin-6-one (186), 8-[1-(3-Fluoro-benzyl)-1H-pyrazol-4-yl]-1-propyl-1,7-dihydro-purin-6-one (187),
2-Morpholin-4-yl-1-propyl-8-[1-(4-trifluoromethyl-benzyl)-1H-pyrazol-4-yl]-1,7-dihydro-purin-6-one (188),
N-(4-Cyano-phenyl)-2-[4-(6-oxo-1-propyl-6, 7-dihydro-1H-purin-8-yl)-phenoxy]-acetamide (189),
[4-(6-Oxo-1-propyl-6,7-dihydro-1H-purin-8-yl)-phenoxy]-acetic acid (190), 8-(1-Benzyl-1H-pyrazol-4-yl)-2-chloro-1-propyl-1,7-dihydro-purin-6-one (191), 8-(4-{2-Oxo-2-[4-(3-trifluoromethyl-phenyl)-piperazin-1-yl]-ethoxy}-phenyl)-1-propyl-1,7-dihydro-purin-6-one (192), 8-(1-Benzyl-1H-pyrazol-4-yl)-1-propyl-2-(4-trifluoromethyl-benzylamino)-1,7-dihydro-purin-6-one (193),
8-(1-Benzyl-1H-pyrazol-4-yl)-1-propyl-2-(3-trifluoromethyl-benzylamino)-1,7-dihydro-purin-6-one (194),
8-(1-Benzyl-1 H-pyrazol-4-yl)-2-[2-(4-methoxy-phenyl)-ethylamino]-1-propyl-1,7-dihydro-purin-6-one (195),
8-(1-Benzyl-1H-pyrazol-4-yl)-2-phenethylamino-1-propyl-1,7-dihydro-purin-6-one (196),
8-(1-Benzyl-1 H-pyrazol-4-yl)-2-(4-methyl-piperazin-1-yl)-1-propyl-1,7-dihydro-purin-6-one (197),
8-(1-Benzyl-1 H-pyrazol-4-yl)-2-piperidin-1-yl-1-propyl-1,7-dihydro-purin-6-one (198),
8-{1-[1-(2,4-Difluoro-phenyl)-5-oxo-pyrrolidin-3-ylmethyl]-1 H-pyrazol-4-yl}-1-propyl-1,7-dihydro-purin-6-one (199),
8-[1-(3-Fluoro-4-trifluoromethyl-benzyl)-1H-pyrazol-4-yl]-2-(2-hydroxy-ethylamino)-1-propyl-1,7-dihydro-purin-6-one (200),
2-Amino-1-propyl-8-[1-(3-trifluoromethyl-benzyl)-1H-pyrazol-4-yl]-1,7-dihydro-purin-6-one (201),
8-(1-Benzyl-1H-pyrazol-4-yl)-2-methylamino-1-propyl-1, 7-dihydro-purin-6-one (202), [8-(1-Benzyl-1H-pyrazol-4-yl)-6-oxo-1-propyl-6, 7-dihydro-1H-purin-2-ylamino]-acetic acid ethyl ester (203),
8-(1-Benzyl-1 H-pyrazol-4-yl)-2-methoxy-1-propyl-1,7-dihydro-purin-6-one (204), 1,2-Dipropyl-8-[1-(3-trifluoromethyl-benzyl)-1 H-pyrazol-4-yl]-1,7-dihydro-purin-6-one (205),
1-Propyl-8-[1-(3-trifluoromethyl-benzyl)-1H-pyrazol-4-yl]-2-(4-trifluoromethyl-phenyl)-1,7-dihydro-purin-6-one (206),
1-Propyl-8-[1-(3-trifluoromethyl-benzyl)-1 H-pyrazol-4-yl]-2-(3-trifluoro methyl-phenyl)-1,7-dihydro-purin-6-one (207),
2-(3-Fluoro-phenyl)-1-propyl-8-[1-(3-trifluoromethyl-benzyl)-1H-pyrazol-4-yl]-1,7-dihydro-purin-6-one (208), 2-Dimethylamino-1-propyl-8-[1-(3-trifluoromethyl-benzyl)-1H-pyrazol-4-yl]-1,7-dihydro-purin-6-one (209),
8-[1-(3-Fluoro-4-trifluoromethyl-benzyl)-1H-pyrazol-4-yl]-6-oxo-1-propyl-6,7-dihydro-1H-purine-2-carbonitrile (210),
8-[1-(3-Fluoro-4-trifluoromethyl-benzyl)-1H-pyrazol-4-yl]-6-oxo-1-propyl-6,7-dihydro-1H-purine-2-carboxylic acid (211),
8-(4-Benzyloxy-phenyl)-1-propyl-2-(3-trifluoromethyl-phenyl)-1,7-dihydro-purin-6-one (212), 8-{4-[3-(4-Fluoro-phenyl)-prop-2-ynyloxy]-phenyl}-1-propyl-2-(3-trifluoromethyl-phenyl)-1,7-dihydro-purin-6-one (213),
8-(4-Methoxy-phenyl)-1-propyl-2-(3-trifluoromethyl-phenyl)-1,7-dihydro-purin-6-one (214),
2-Ethyl-1-propyl-8-[1-(3-trifluoromethyl-benzyl)-1 H-pyrazol-4-yl]-1,7-dihydro-purin-6-one (215),
2-Benzyl-1-propyl-8-[1-(3-trifluoromethyl-benzyl)-1H-pyrazol-4-yl]-1,7-dihydro-purin-6-one (216),
{6-Oxo-1-propyl-8-[1-(3-trifluoromethyl-benzyl)-1H-pyrazol-4-yl]-6, 7-dihydro-1H-purin-2-ylamino}-acetic acid (217),
(S)-1-{6-Oxo-1-propyl-8-[1-(3-trifluoromethyl-benzyl)-1H-pyrazol-4-yl]-6,7-dihydro-1H-purin-2-yl}-pyrrolidine-2-carboxylic acid (218),
1-Propyl-2-pyrrolidin-1-yl-8-[1-(3-trifluoromethyl-benzyl)-1 H-pyrazol-4-yl]-1,7-dihydro-purin-6-one (219),
2-Methylamino-1-prop yl-8-[1-(3-trifluoromethyl-benzyl)-1 H-pyrazol-4-yl]-1,7-dihydro-purin-6-one (220),
2-Cyclobutylamino-1-propyl-8-[1-(3-trifluoromethyl-benzyl)-1 H-pyrazol-4-yl]-1,7-dihydro-purin-6-one (221),
2-Chloro-8-[1-(3-fluoro-4-trifluoromethyl-benzyl)-1H-pyrazol-4-yl]-7-methyl-1-propyl-1,7-dihydro-purin-6-one (222),
2-Methoxy-1-propyl-8-[1-(3-trifluoromethyl-benzyl)-1 H-pyrazol-4-yl]-1,7-dihydro-purin-6-one (223), 6-Oxo-1-propyl-8-[1-(3-trifluoromethyl-benzyl)-1H-pyrazol-4-yl]-6, 7-dihydro-1H-purine-2-carbonitrile (224),
2-Cyclopentyloxy-1-propyl-8-[1-(3-trifluoromethyl-benzyl)-1 H-pyrazol-4-yl]-1,7-dihydro-purin-6-one (225),
6-Oxo-1-propyl-8-[1-(3-trifluoromethyl-benzyl)-1H-pyrazol-4-yl]-6, 7-dihydro-1H-purine-2-carboxylic acid amide (226),
{6-Oxo-1-propyl-8-[1-(3-trifluoromethyl-benzyl)-1H-pyrazol-4-yl]-6, 7-dihydro-1H-purin-2-yloxy }-acetic acid ethyl ester (227),
2-Morpholin-4-yl-1-propyl-8-[1-(3-trifluoromethyl-benzyl)-1H-pyrazol-4-yl]-1,7-dihydro-purin-6-one (228),
{6-Oxo-1-propyl-8-[1-(3-trifluoromethyl-benzyl)-1H-pyrazol-4-yl]-6, 7-dihydro-1H-purin-2-yloxy }-acetic acid (229),
8-{1-[3-(4-Fluoro-phenyl)-prop-2-ynyl]-1 H-pyrazol-4-yl}-1-propyl-2-pyrrolidin-1-yl-1,7-dihydro-purin-6-one (230),
(S)-1-{6-Oxo-1-propyl-8-[1-(3-trifluoromethyl-benzyl)-1H-pyrazol-4-yl]-6,7-dihydro-1H-purin-2-yl}-pyrrolidine-2-carboxylic acid amide (231), 1-{6-Oxo-1-propyl-8-[1-(3-trifluoromethyl-benzyl)-1H-pyrazol-4-yl]-6,7-dihydro-1H-purin-2-yl}-piperidine-3-carboxylic acid (232),
1-{6-Oxo-1-propyl-8-[1-(3-trifluoromethyl-benzyl)-1H-pyrazol-4-yl]-6,7-dihydro-1H-purin-2-yl}-piperidine-4-carboxylic acid (233),
(2R,4R)-4-Hydroxy-1-{6-oxo-1-propyl-8-[1-(3-trifluoromethyl-benzyl)-1H-pyrazol-4-yl]-6,7-dihydro-1H-purin-2-yl}-pyrrolidine-2-carboxylic acid (234), 2-(2,3-Dihydroxy-propylamino)-1-propyl-8-[1-(3-trifluoromethyl-benzyl)-1H-pyrazol-4-yl]-1,7-dihydro-purin-6-one (235),
2-(2-Methoxy-ethylamino)-1-propyl-8-[1-(3-trifluoromethyl-benzyl)-1H-pyrazol-4-yl]-1,7-dihydro-purin-6-one (236),
2-(4-Hydroxy-piperidin-1-yl)-1-propyl-8-[1-(3-trifluoromethyl-benzyl)-1H-pyrazol-4-yl]-1,7-dihydro-purin-6-one (237),
2-(3-Hydroxy-piperidin-1-yl)-1-propyl-8-[1-(3-trifluoromethyl-benzyl)-1H-pyrazol-4-yl]-1,7-dihydro-purin-6-one (238), 2-{6-Oxo-1-propyl-8-[1-(3-trifluoromethyl-benzyl)-1H-pyrazol-4-yl]-6,7-dihydro-1H-purin-2-ylamino}-ethanesulfonic acid (239),
2-(3-Hydroxymethyl-piperidin-1-yl)-1-propyl-8-[1-(3-trifluoromethyl-benzyl)-1H-pyrazol-4-yl]-1,7-dihydro-purin-6-one (240),
(Methyl-{6-oxo-1-propyl-8-[1-(3-trifluoromethyl-benzyl)-1H-pyrazol-4-yl]-6, 7-dihydro-1H-purin-2-yl}-amino)-acetic acid (241),
2-(2-Hydroxy-ethylamino)-1-propyl-8-[1-(3-trifluoromethyl-benzyl)-1H-pyrazol-4-y1]-1,7-dihydro-purin-6-one (242),
2-(4-Hydroxymethyl-piperidin-1-yl)-1-propyl-8-[1-(3-trifluoromethyl-benzyl)-1H-pyrazol-4-yl]-1,7-dihydro-purin-6-one (243), 2-(4-Hydroxymethyl-piperidin-1-yl)-1-propyl-8-[1-(3-trifluoromethyl-benzyl)-1H-pyrazol-4-yl]-1,7-dihydro-purin-6-one (244),
(S)-3-Methyl-2-{6-oxo-1-propyl-8-[1-(3-trifluoromethyl-benzyl)-1H-pyrazol-4-yl]-6.7-dihydro-1H-purin-2-ylamino}-butyric acid (245),
2-((S)-2-Methoxymethyl-pyrrolidin-1-yl)-1-propyl-8-[1-(3-trifluoromethyl-benzyl)-1H-pyrazol-4-yl]-1,7-dihydro-purin-6-one (246),
2-((S)-2-Hydroxymethyl-pyrrolidin-1-yl)-1-propyl-8-[1-(3-trifluoromethyl-benzyl)-1H-pyrazol-4-yl]-1,7-dihydro-purin-6-one (247),
2-((R)-3-Hydroxy-pyrrolidin-1-yl)-1-propyl-8-[1-(3-trifluoromethyl-benzyl)-1H-pyrazol-4-yl]-1,7-dihydro-purin-6-one (248),
1-Propyl-2-(tetrahydro-pyran-4-ylamino)-8-[1-(3-trifluoromethyl-benzyl)-1H-pyrazol-4-yl]-1,7-dihydro-purin-6-one (249),
2-Fluoro-1-propyl-8-[1-(3-trifluoromethyl-benzyl)-1 H-pyrazol-4-yl]-1,7-dihydro-purin-6-one (250),
1-Propyl-2-(2,2,2-trifluoro-ethoxy)-8-[1-(3-trifluoromethyl-benzyl)-1H-pyrazol-4-y1]-1,7-dihydro-purin-6-one (251),
2-(2-Methoxy-ethoxy)-1-propyl-8-[1-(3-trifluoromethyl-benzyl)-1H-pyrazol-4-yl]-1.7-dihydro-purin-6-one (252),
7-Methyl-1-propyl-8-[1-(3-trifluoromethyl-benzyl)-1 H-pyrazol-4-yl]-1,7-dihydro-purin-6-one (253), 2-Chloro-1-propyl-8-[6-(3-trifluoromethyl-benzylamino)-pyridin-3-yl]-1,7-dihydro-purin-6-one (254),
2-Chloro-8-[6-(3-fluoro-benzylamino)-pyridin-3-yl]-1-propyl-1,7-dihydro-purin-6-one (255),
1-Propyl-8-[6-(3-trifluoromethyl-benzylamino)-pyridin-3-yl]-1,7-dihydro-purin-6-one (256),
1-Propyl-8-(1-pyridin-3-ylmethyl-1H-pyrazol-4-yl)-1,7-dihydro-purin-6-one (257), 1-Propyl-8-[1-(6-trifluoromethyl-pyridin-3-ylmethyl)-1 H-pyrazol-4-yl]-1,7-dihydro-purin-6-one (258),
2-Cyclopropyl-1-propyl-8-[1-(3-trifluoromethyl-benzyl)-1 H-pyrazol-4-yl]-1,7-dihydro-purin-6-one (259),
2-Difluoromethoxy-1-propyl-8-[1-(3-trifluoromethyl-benzyl)-1 H-pyrazol-4-yl]-1,7-dihydro-purin-6-one (260), 1-Propyl-2-trifluoromethyl-8-[1-(3-trifluoromethyl-benzyl)-1 H-pyrazol-4-yl]-1,7-dihydro-purin-6-one (261),
2-Chloro-8-(1-methyl-1H-pyrazol-4-yl)-1-propyl-1,7-dihydro-purin-6-one (262), 8-(1-Methyl-1H-pyrazol-4-yl)-1-propyl-1,7-dihydro-purin-6-one (263),
2-Isobutylamino-8-(1-methyl-1H-pyrazol-4-yl)-1-propyl-1,7-dihydro-purin-6-one (264),
8-(1-Methyl-1 H-pyrazol-4-yl)-1-propyl-2-pyrrolidin-1-yl-1,7-dihydro-purin-6-one (265),
2-[2-(4-Methoxy-phenyl)-ethylamino]-8-(1-methyl-1H-pyrazol-4-yl)-1-propyl-1,7-dihydro-purin-6-one (266),
2-(4-Methyl-piperazin-1-yl)-8-(1-methyl-1 H-pyrazol-4-yl)-1-propyl-1,7-dihydro-purin-6-one (267),
2-[4-(4-Fluoro-phenyl)-piperazin-1-yl]-8-(1-methyl-1H-pyrazol-4-yl)-1-propyl-1,7-dihydro-purin-6-one (268),
1-[8-(1-Methyl-1H-pyrazol-4-yl)-6-oxo-1-propyl-6, 7-dihydro-1H-purin-2-yl]-pyrrolidine-2-carboxylic acid methyl ester (269),
2-Benzyl-8-(1-methyl-1H-pyrazol-4-yl)-1-propyl-1,7-dihydro-purin-6-one (270), 2-(3-Fluoro-phenyl)-8-(1-methyl-1 H-pyrazol-4-yl)-1-propyl-1,7-dihydro-purin-6-one (271),
8-(1-Methyl-1H-pyrazol-4-yl)-1-propyl-2-(4-trifluoromethyl-phenyl)-1,7-dihydro-purin-6-one (272),
8-(1-Methyl-1H-pyrazol-4-yl)-2-phenethylamino-1-propyl-1,7-dihydro-purin-6-one (273),
8-(1-Methyl-1H-pyrazol-4-yl)-1-propyl-2-(4-trifluoromethyl-benzylamino)-1,7-dihydro-purin-6-one (274),
2-Cycloprop ylamino-8-(1-methyl-1 H-pyrazol-4-yl)-1-propyl-1,7-dihydro-purin-6-one (275),
2-(3-Fluoro-phenoxy)-8-(1-methyl-1H-pyrazol-4-yl)-1-propyl-1,7-dihydro-purin-6-one (276),
2-(4-Methoxy-phenylamino)-8-(1-methyl-1H-pyrazol-4-yl)-1-propyl-1, 7-dihydro-purin-6-one (277),
7-Benzyl-2-chloro-8-(1-methyl-1H-pyrazol-4-yl)-1-propyl-1,7-dihydro-purin-6-one (278),
9-Benzyl-2-chloro-8-(1-methyl-1H-pyrazol-4-yl)-1-propyl-1,9-dihydro-purin-6-one (279),
2-Amino-7-benzyl-8-(1-methyl-1H-pyrazol-4-yl)-1-propyl-1, 7-dihydro-purin-6-one (280),
2-Chloro-8-furan-2-yl-1-propyl-1,7-dihydro-purin-6-one (281),
2-Amino-8-[1-(4-fluoro-benzyl)-1 H-imidazo [1,2-b]pyrazol-7-yl]-1-propyl-1,7-dihydro-purin-6-one (282),
2-Chloro-8-[1-(4-fluoro-benzyl)-1 H-imidazo [1,2-b]pyrazol-7-yl]-1-propyl-1,7-dihydro-purin-6-one (283),
2-Amino-8-(1-methyl-1H-pyrazol-4-yl)-1-propyl-1,7-dihydro-purin-6-one (284), 2-Amino-7-methyl-8-(1-methyl-1H-pyrazol-4-yl)-1-propyl-1, 7-dihydro-purin-6-one (285),
2-Amino-9-methyl-8-(1-methyl-1H-pyrazol-4-yl)-1-propyl-1, 9-dihydro-purin-6-one (286),
7-Methyl-8-(1-methyl-1 H-pyrazol-4-yl)-1-propyl-1,7-dihydro-purin-6-one (287), 9-Methyl-8-(1-methyl-1H-pyrazol-4-yl)-1-propyl-1,9-dihydro-purin-6-one (288),
2-Amino-8-furan-2-yl-1-propyl-1,7-dihydro-purin-6-one (289),
2-Chloro-8-furan-2-yl-7-methyl-1-propyl-1,7-dihydro-purin-6-one (290), 8-(1-Methyl-1H-pyrazol-4-yl)-1-propyl-2-(3-trifluoromethyl-benzylamino)-1,7-dihydro-purin-6-one (291),
2-Furan-2-yl-8-(1-methyl-1 H-pyrazol-4-yl)-1-propyl-1,7-dihydro-purin-6-one (292),
8-(1-Benzyl-1H-pyrazol-4-yl)-2-furan-2-yl-1-propyl-1,7-dihydro-purin-6-one (293),
2-Chloro-8-(6-chloro-pyridin-3-yl)-1-propyl-1,7-dihydro-purin-6-one (294),
2-Difluoromethyl-1-propyl-8-[1-(3-trifluoromethyl-benzyl)-1 H-pyrazol-4-yl]-1,7-dihydro-purin-6-one (295),
2-Fluoromethyl-1-propyl-8-[1-(3-trifluoromethyl-benzyl)-1 H-pyrazol-4-yl]-1,7-dihydro-purin-6-one (296),
2-Fluoromethyl-8-{1-[3-(3-methoxy-phenyl)-prop-2-ynyl]-1H-pyrazol-4-yl}-1-propyl-1,7-dihydro-purin-6-one (297),
2-Difluoromethyl-8-{1-[2-oxo-2-(4-m-tolyl-piperazin-1-yl)-ethyl]-1 H-pyrazol-4-yl}-1-propyl-1,7-dihydro-purin-6-one (298),
3-Fluoro-N-methyl-N-[5-(6-oxo-1-propyl-6, 7-dihydro-1H-purin-8-yl)-pyridin-2-yl]-benzamide (299),
N-[5-(2-Difluoromethyl-6-oxo-1-propyl-6, 7-dihydro-1H-purin-8-yl)-pyridin-2-yl]-3-methoxy-N-methyl-benzamide (300),
N-[5-(2-Difluoromethyl-6-oxo-1-propyl-6, 7-dihydro-1H-purin-8-yl)-pyridin-2-yl]-3-methoxy-benzenesulfonamide (301),
2-Fluoromethyl-1-propyl-8-[1-(5-trifluoromethyl-pyridin-3-ylmethyl)-1H-pyrazol-4-yl]-1,7-dihydro-purin-6-one (302),
2-Fluoromethyl-1-propyl-8-[1-(2-trifluoromethyl-pyridin-4-ylmethyl)-1H-pyrazol-4-yl]-1,7-dihydro-purin-6-one (303),
2-Fluoromethyl-8-[3-(3-methoxy-phenoxy)-isoxazol-5-yl]-1-propyl-1, 7-dihydro-purin-6-one (304), 2-Difluoromethyl-8-{3-[3-(3-fluoro-phenyl)-prop-2-ynyloxy]-isoxazol-5-yl}-1-propyl-1,7-dihydro-purin-6-one (305),
2-Fluoromethyl-1-(2-hydroxy-ethyl)-8-[3-(3-methoxy-phenoxy)-isoxazol-5-yl]-1,7-dihydro-purin-6-one (306),
2-Difluoromethyl-1-ethyl-8-{3-[3-(3-fluoro-phenyl)-prop-2-ynyloxy]-isoxazol-5-yl}-1,7-dihydro-purin-6-one (307),
2-Difluoromethyl-1-ethyl-8-(1-{2-[4-(3-methoxy-phenyl)-piperazin-1-yl]-2-oxo-ethyl}-1H-pyrazol-4-yl)-1,7-dihydro-purin-6-one (308),
1-Ethyl-8-(1-{2-[4-(3-methoxy-phenyl)-piperazin-1-yl]-2-oxo-ethyl}-1 H-pyrazol-4-y1)-6-oxo-6, 7-dihydro-1H-purine-2-carbonitrile (309),
N-[5-(2-Cyano-6-oxo-1-propyl-6, 7-dihydro-1H-purin-8-yl)-pyridin-2-yl]-3-methoxy-benzenesulfonamide (310),
N-{5-[2-Cyano-1-(2-hydroxy-ethyl)-6-oxo-6, 7-dihydro-1H-purin-8-yl]-pyridin-2-yl}-3-methoxy-benzenesulfonamide (311), 2-Difluoromethyl-1-ethyl-8-{4-[3-(3-methoxy-phenyl)-prop-2-ynyloxy]-phenyl}-1,7-dihydro-purin-6-one (312),
2-Difluoromethyl-1-ethyl-8-{4-[1-(3-fluoro-phenyl)-5-oxo-pyrrolidin-3-ylmethoxy]-phenyl}-1,7-dihydro-purin-6-one (313),
2-Difluoromethyl-8-[5-(3-methoxy-phenoxy)-1-methyl-1 H-pyrazol-3-yl]-1-propyl-1,7-dihydro-purin-6-one (314),
2-Difluoromethyl-8-{5-[1-(3-methoxy-phenyl)-piperidin-4-yloxy]-1-methyl-1 H-pyrazol-3-yl}-1-propyl-1,7-dihydro-purin-6-one (315),
2-Fluoromethyl-8-{3-[1-(3-fluoro-phenyl)-piperidin-4-yloxy]-isoxazol-5-yl}-1-propyl-1,7-dihydro-purin-6-one (316),
1-Ethyl-8-{6-[1-(3-fluoro-phenyl)-5-oxo-pyrrolidin-3-ylmethoxy]-pyridin-3-yl}-6-oxo-6, 7-dihydro-1H-purine-2-carbonitrile (317),
1-Ethyl-8-{6-[1-(3-methoxy-phenyl)-pyrrolidin-3-yloxy]-pyridin-3-yl}-6-oxo-6,7-dihydro-1 H-purine-2-carbonitrile (318),
3-[4-(2-Difluoromethyl-1-ethyl-6-oxo-6, 7-dihydro-1H-purin-8-yl)-pyrazol-1-ylmethyl]-benzoic acid (319), 2-Difluoromethyl-1-ethyl-8-[1-(3-hydroxymethyl-benzyl)-1 H-pyrazol-4-yl]-1,7-dihydro-purin-6-one (320),
2-Difluoromethyl-3-ethyl-6-[1-(3-methoxy-benzyl)-1 H-pyrazol-4-yl]-3 ,5-dihydro-pyrrolo[3,2-d]pyrimidin-4-one (321),
N-[5-(2-Cyano-4-oxo-3-propyl-4,5-dihydro-3H-pyrrolo[3,2-d]pyrimidin-6-yl)-pyridin-2-yl]-3-methoxy-benzenesulfonamide (322), 2-Fluoromethyl-6-{3-[1-(3-fluoro-phenyl)-piperidin-4-yloxy]-isoxazol-5-yl}-3-propyl-3, 5-dihydro-pyrrolo[3,2-d]pyrimidin-4-one (323),
2-Difluoromethyl-6-{5-[1-(3-methoxy-phenyl)-piperidin-4-yloxy]-1-methyl-1H-pyrazol-3-yl}-3-propyl-3,5-dihydro-pyrrolo[3,2-d]pyrimidin-4-one (324),
3-Ethyl-6-{6-[1-(3-methoxy-phenyl)-pyrrolidin-3-yloxy]-pyridin-3-yl}-4-oxo-4,5-dihydro-3H-pyrrolo[3,2-d]pyrimidine-2-carbonitrile (325),
2-Fluoromethyl-6-{3-[1-(3-fluoro-phenyl)-piperidin-4-yloxy]-isoxazol-5-yl}-7-hydroxy-3-propyl-3,5-dihydro-pyrrolo[3,2-d]pyrimidin-4-one (326),
2-Difluoromethyl-3-ethyl-6-[1-(3-methoxy-benzyl)-1H-pyrazol-4-yl]-7-methyl-3,5-dihydro-pyrrolo[3,2-d]pyrimidin-4-one (327),
2-Difluoromethyl-1-ethyl-8-[1-(3-methoxy-benzyl)-1 H-pyrazol-4-yl]-7-methyl-1,7-dihydro-purin-6-one (328),

N-[5-(2-Cyano-7-methyl-6-oxo-1-propyl-6, 7-dihydro-1H-purin-8-yl)-pyridin-2-yl]-3-methoxy-benzenesulfonamide (329),
1-(2,2-Difluoro-ethyl)-2-ethyl-8-[1-(3-methoxy-benzyl)-1H-pyrazol-4-yl]-1,7-dihydro-purin-6-one (330),
3-{3-[4-(2-Difluoromethyl-6-oxo-1-propyl-6, 7-dihydro-1H-purin-8-yl)-pyrazol-1-yl]-prop-1-ynyl}-benzoic acid (331), 3-(3-{4-[1-(2,2-Difluoro-ethyl)-2-ethyl-6-oxo-6,7-dihydro-1 H-purin-8-yl]-pyrazol-1-yl}-prop-1-ynyl)-benzoic acid (332),
3-{3-[4-(6-Oxo-1-propyl-2-trifluoromethyl-6,7-dihydro-1H-purin-8-yl)-pyrazol-1-yl]-prop-1-ynyl}-benzoic acid (333), and
6-Oxo-1-propyl-8-[6-(3-trifluoromethyl-benzyl)-pyridin-3-yl]-6,7-dihydro-1H-purine-2-carbonitrile (334).

In an embodiment of the present disclosure, there is provided a pharmaceutical composition comprising compound of Formula III or IV, its pharmaceutically acceptable salts, analogs, tautomeric forms, stereoisomers, geometrical isomers, polymorphs, hydrates, solvates, metabolites, and prodrugs thereof

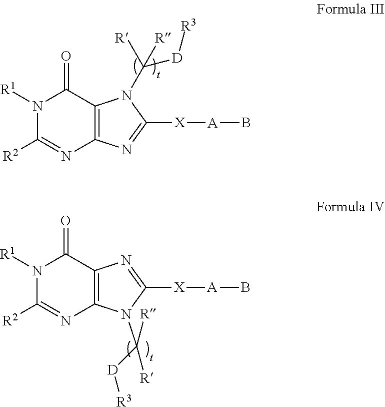

wherein,
R1 is an alkyl wherein one or more methylene groups are optionally replaced by hetero atoms or group selected from -0-, —S(0)p-, —N(Ra)—, or —C(O), provided that the heteroatom is not adjacent to N in the ring; p is selected from 0, 1 or 2;
wherein alkyl is unsubstituted or substituted with alkoxy, acyl, acylamino, acyloxy, amino, monoalkylamino, dialkylamino, aminocarbonyl, alkoxycarbonylamino, azido, cyano, halogen, haloalkyl, hydroxy, hydroxyalkyl, kcto, thiocarbonyl, carboxy, alkylcarboxy, carboxyalkyl, -aminocarbonylamino, hydroxyamino, alkoxy amino;
R2 is selected from the group consisting of hydrogen, halogen, cyano, nitro, carboxy, acyl, aminocarbonyl, alkyl, alkonyl, alkynyl, hydroxyalkyl, carboxyalkyl, haloalkyl, haloalkyloxy, alkoxy, —NRb Rb, —S(0)p Rb, cycloalkyl, cycloalkylalkyl, cycloalkyloxy, aryl, arylalkyl, aryloxy, heterocyclyl, heterocyclylalkyl, heterocyclyloxy, heteroaryl, heteroarylalkyl and heteroaryloxy; wherein alkyl, alkenyl, alkynyl, alkoxy, carboxyalkyl, cycloalkyl, cycloalkylalkyl, cycloalkyloxy, aryl, arylalkyl, aryloxy, heterocyclyl, heterocyclylalkyl, heterocyclyloxy, heteroaryl, heteroarylalkyl, heteroaryloxy and Rb are unsubstituted or substituted independently with alkyl, alkenyl, alkynyl, alkoxy, acyl, acylamino, acyloxy, nitro, amino, monoalkylamino, dialkylamino, hydroxyamino, alkoxyamino, aminocarbonylamino, azido, cyano, halogen, hydroxy, hydroxyalkyl, kcto, thiocarbonyl, carboxy, alkylcarboxy, carboxyalkyl, —SO3H, arylamino, cycloalkylamino, heteroarylamino, heterocyclylamino, aminocarbonyl, alkoxycarbonylamino, cycloalkyl, cycloalkyloxy, cycloalkenyl, aryl, aryloxy, heteroaryl, heteroaryloxy, heterocyclyl, heterocyclyloxy, —S(0)2 NRc Rc, —NRc S(0)2 Rc or —S(0)p Rd; wherein cach substituent is unsubstituted or substituted with 1, 2, or 3 substituents independently selected from alkyl, carboxy, carboxyalkyl, aminocarbonyl, hydroxy, alkoxy, halogen, haloalkyl, haloalkoxy, amino, substituted amino, cyano or —S(0)p Rd;
R′ and R″ are independently selected from hydrogen, or alkyl; or
R′ and R″ taken together may represent O, or a lower cycloalkyl ring system which is saturated or partially unsaturated;
R3 is selected from the group consisting of alkyl, aryl, —C(0)R4 and -P(0)(OR5)2;
R4 is selected from alkyl, alkoxy, aryl, heteroaryl, heterocyclyl, or -NR6 R7;
R5 is selected from hydrogen, alkyl, aryl, arylalkyl, —CH2 OC(0)alkyl, or —CH2 0C(0)Oalkyl; or two R5 groups taken together form a five or six membered ring system which is saturated or partially unsaturated and is optionally substituted with 1 to 4 substituents independently selected from halo, alkyl, aryl or heteroaryl; R6 and R7 are independently selected from the group consisting of hydrogen, alkyl, heterocyclyl and heterocyclylalkyl; or
R6 and R7 taken together form a monocyclic ring system which is saturated or partially unsaturated and optionally have additional heteroatoms selected from O, N or S, wherein the ring system is optionally substituted with 1 to 4 substituents independently selected from halo, alkyl, alkoxy, or -NR8 R9;
R4, R5, R6 and R7 is optionally substituted with 1 to 4 substituents independently selected from hydroxyl, halogen, alkyl, alkoxy, haloalkyl, -NR8 R9, —C(0)OR10, —OC(0)R10 or -NC(0)R10;
R8 and R9 are independently selected from the group consisting of hydrogen and alkyl; R10 is selected from hydrogen, hydroxy, halogen, amino, substituted amino, cyano, alkyl, alkoxy, haloalkyl, haloalkoxy, carboxy, carboxyalkyl, aminocarbonyl, aryl or arylalkyl;
X is an optionally substituted arylene or an optionally substituted heteroarylene;
A is selected from a bond, or (Ci-C6)alkylene, wherein 1 to 4 methylene groups are optionally replaced by group independently selected from O, —S(0)p-, —N(Rb)—, or —C(O)—; wherein alkylene is unsubstituted or substituted independently with alkyl, alkenyl, alkynyl, alkoxy, cycloalkyl, cycloalkenyl, acyl, acylamino, acyloxy, amino, monoalkylamino, dialkylamino, arylamino, cycloalkylamino, heteroarylamino, heterocyclylamino, aminocarbonyl, alkoxycarbonylamino, azido, cyano, halogen, hydroxy, hydroxyalkyl, kcto, thiocarbonyl, carboxy, alkylcarboxy, carboxyalkyl, carboxyalkyloxy, alkylcarboxyalkyloxy —S03 H, aryl, aryloxy, cycloalkyloxy, hctcroaryl, aminocarbonylamino, heteroaryloxy, heterocyclyl, heterocyclyloxy, hydroxyamino, alkoxyamino, nitro, S(0)2 NRc Rc, —NRc S(0)2 Rc or —S(0)p Rd;
wherein cach substituent is unsubstituted or substituted with 1, 2, or 3 substituents independently selected from alkyl, carboxy, carboxyalkyl, aminocarbonyl, hydroxy, alkoxy, halogen, CF3, amino, substituted amino, cyano or —S(0)p Rd;
B is selected from hydrogen, heterocyclyl, cycloalkyl, aryl or heteroaryl; wherein heterocyclyl, cycloalkyl, aryl and heteroaryl are unsubstituted or substituted independently with alkyl, alkenyl, alkynyl, alkoxy, cycloalkyl, cycloalkylalkyl, cycloalkonyl, acyl, acylamino, acyloxy, amino, monoalkylamino, dialkylamino, arylamino, cycloalkylamino, heteroarylamino, heterocyclylamino, aminocarbonyl, alkoxycarbonylamino, azido, cyano, halogen, hydroxy, hydroxyalkyl, kcto, thiocarbonyl, carboxy, alkylcarboxy, carboxyalkyl, carboxyalkyloxy, alkylcarboxyalkyloxy—SO3H, aryl, arylalkyl, aryloxy, cycloalkyloxy, heteroaryl, heteroarylalkyl, aminocarbonylamino, heteroaryloxy, heterocyclyl, heterocyclylalkyl, heterocyclyloxy, hydroxyamino, alkoxyamino, nitro, S(0)2 NRb Rb, —NRb S(0)2 Rb or —S(0)p Rd; wherein cach substituent is unsubstituted or substituted with 1, 2, or 3 substituents independently selected from alkyl, carboxy, carboxyalkyl, aminocarbonyl, hydroxy, alkoxy, alkoxyalkoxy, alkoxyalkyl, halogen, haloalkyl, haloalkoxy, amino, substituted amino, cyano or —S(0)P Rd;
D is selected from -0-, —S(0)p-, or —N(Ra)—;
Ra is hydrogen or an alkyl;
Rb is selected from the group consisting of hydrogen, alkyl, acyl, carboxyalkyl, carbonylamino, cycloalkyl, cycloalkylalkyl, aryl, arylalkyl, heteroaryl, hctcroarylalkyl, heterocyclyl and heterocyclylalkyl;
Rc is selected from hydrogen, alkyl, aryl, heteroaryl or heterocyclyl;
Rd is selected from alkyl, cycloalkyl, aryl, heterocyclyl or heteroaryl;
p is 0, 1 or 2; and
t is 1 or 2, for the manufacture of a medicament for the treatment of a condition or disorder ameliorated by inhibition of the A2A/A2B receptor.

In an embodiment of the present disclosure, there is provided a pharmaceutical composition comprising compound of Formula III or IV, its pharmaceutically acceptable salts, analogs, tautomeric forms, stereoisomers, geometrical isomers, polymorphs, hydrates, solvates, metabolites, and prodrugs thereof, wherein
R1 is an alkyl;
R2 is selected from the group consisting of hydrogen, halogen, cyano, nitro, alkyl, hydroxyalkyl, haloalkyl, haloalkyloxy and alkoxy;
R′ and R″ are independently selected from hydrogen or alkyl;
R3 is selected from the group consi .-tuig of alkyl. —C(0)R4 and -P(0)(OR5)2;
R4 is selected from alkyl or alkoxy;
R5 is selected from the group consisting of hydrogen, alkyl, —CH2 OC(0)alkyl or —CH2 OC(0)Oalkyl;
R4 and R5 is optionally substituted with 1 to 4 substituents independently selected from hydroxyl, halogen, alkyl, alkoxy, haloalkyl, -NR8 R9, —C(0)OR10, —OC(0)R10 or —NC(0)R10;
R° and R* are independently selected from the group consisting of hydrogen and alkyl; R10 is selected from the group consisting of hydrogen, hydroxy, halogen, amino, substituted amino, cyano, alkyl, alkoxy, haloalkyl, haloalkoxy, carboxy, carboxyalkyl, aminocarbonyl, aryl and arylalkyl;
X is optionally substituted heteroarylene;
A is selected from a bond or (Ci-C6)a′kyiene;
B is selected from aryl or heteroaryl, wherein aryl and heteroaryl are unsubstituted or substituted independently with alkyl, alkoxy, acyl, acylamino, acyloxy, amino, monoalkylamino, dialkylamino, aminocarbonyl, alkoxycarbonylamino, azido, cyano, halogen, hydroxy, hydroxyalkyl, haloalkyl, perhaloalkyl, keto, thiocarbonyl, carboxy, alkylcarboxy, carboxyalkyl, carboxyalkyloxy, alkylcarboxyalkyloxy—S03H, aminocarbonylamino, hydroxyamino, alkoxyamino or nitro;
D is selected from -0-, —S (O) p-or —N(Ra)—;
Ra is hydrogen or an alkyl;
p is 0, 1 or 2; and
t is 1 or 2, for the manufacture of a medicament for the treatment of a condition or disorder ameliorated by inhibition of the A2 A/A2B receptor.In an embodiment of the present disclosure, there is provided a pharmaceutical composition comprising compound of Formula III or IV, its pharmaceutically acceptable salts, analogs, tautomeric forms, stereoisomers, geometrical isomers, polymorphs, hydrates, solvates, metabolites, and prodrugs thereof, for use in the treatment of a condition or disorder selected from prostate cancer, rectal cancer, renal cancer, ovarian cancer, endometrial cancer, thyroid cancer, pancreatic cancer, breast cancer, colon cancer, bladder cancer, brain cancer, glial cancer, melanoma cancer, pineal gland cancer, or lung cancer.

In an embodiment of the present disclosure, there is provided a method of using the pharmaceutical composition comprising compound of Formula III or IV, its pharmaceutically acceptable salts, analogs, tautomeric forms, stereoisomers, geometrical isomers, polymorphs, hydrates, solvates, metabolites, and prodrugs thereof, in the treatment of a disease or condition in a mammal that is amenable to treatment with an A2 A/A2B receptor antagonist, the method comprising: administering to a mammal in need thereof a therapeutically effective dose of the pharmaceutical composition comprising compound of Formula III or IV, its pharmaceutically acceptable salts, analogs, tautomeric forms, stereoisomers, geometrical isomers, polymorphs, hydrates, solvates, metabolites, and prodrugs thereof.

In an embodiment of the present disclosure, there is provided a method of treatment of a disorder or condition ameliorated by antagonizing the A2 A receptor, the method comprising: administering an effective amount of the pharmaceutical composition comprising compound of Formula III or IV, its pharmaceutically acceptable salts, analogs, tautomeric forms, stereoisomers, geometrical isomers, polymorphs, hydrates, solvates, metabolites, and prodrugs thereof, to a patient in need of such treatment.

In an embodiment of the present disclosure, there is provided a use of the pharmaceutical composition comprising compound of Formula III or IV, its pharmaceutically acceptable salts, analogs, tautomeric forms, stereoisomers, geometrical isomers, polymorphs, hydrates, solvates, metabolites, and prodrugs thereof, for the preparation of a medicament for the treatment of a condition or disorder selected from prostate cancer, rectal cancer, renal cancer, ovarian cancer, endometrial cancer, thyroid cancer, pancreatic cancer, breast cancer, colon cancer, bladder cancer, brain cancer, glial cancer, melanoma cancer, pineal gland cancer, or lung cancer.

In an embodiment of the present disclosure, there is provided a pharmaceutical composition comprising compound of Formula III or IV, its pharmaceutically acceptable salts, analogs, tautomeric forms, stereoisomers, geometrical isomers, polymorphs, hydrates, solvates, metabolites, and prodrugs thereof, in combination with at least one PD-LI antibody for use in the treatment of a condition or disorder selected from prostate cancer, rectal cancer, renal cancer, ovarian cancer, endometrial cancer, thyroid cancer, pancreatic cancer, breast cancer, colon cancer, bladder cancer, brain cancer, glial cancer, melanoma cancer, pineal gland cancer, or lung cancer.

In an embodiment of the present disclosure, there is provided a use of the pharmaceutical composition comprising compound of Formula III or IV, its pharmaceutically acceptable salts, analogs, tautomeric forms, stereoisomers, geometrical isomers, polymorphs, hydrates, solvates, metabolites, and prodrugs thereof, in combination with at least one PD-LI antibody for use in the treatment of a condition or disorder selected from prostate cancer, rectal cancer, renal cancer, ovarian cancer, endometrial cancer, thyroid cancer, pancreatic cancer, breast cancer, colon cancer, bladder cancer, brain cancer, glial cancer, melanoma cancer, pineal gland cancer, or lung cancer.

In an embodiment of the present disclosure, there is provided a pharmaceutical composition comprising compound of Formula III or IV, its pharmaceutically acceptable salts, analogs, tautomeric forms, stereoisomers, geometrical isomers, polymorphs, hydrates, solvates, metabolites, and prodrugs thereof, wherein the compound of Formula III or Formula IV is selected from the group consisting of:
Phosphoric acid mono-{2-cyano-6-oxo-1-propyl-8-[1-(3-trifluoromethyl-benzyl)-1H-pyrazol-4-y1]-1,6-dihydro-purin-7-ylmethyl} ester (335),
Phosphoric acid mono-{2-cyano-6-oxo-1-propyl-8-[1-(3-trifluoromethyl-benzyl)-1H-pyrazol-4-yl]-1,6-dihydro-purin-9-ylmethyl} ester di sodium salt (336),
Phosphoric acid mono-{2-chloro-6-oxo-1-propyl-8-[1-(3-trifluoromethyl-benzyl)-1 H-pyrazol-4-y1]-1,6-dihydro-purin-7-ylmethyl} ester (337),
Phosphoric acid mono-{2-chloro-6-oxo-1-propyl-8-[1-(3-trifluoromethyl-benzyl)-1H-pyrazol-4-yl]-1,6-dihydro-purin-9-ylmethyl} ester (338),
2,2-Dimethyl-propionic acid 6-oxo-1-propyl-8-[1-(3-trifluoromethyl-benzyl)-1H-pyrazol-4-yl]-1,6-dihydro-purin-7-ylmethyl ester (339),
2,2-Dimethyl-propionic acid 6-oxo-1-propyl-8-[1-(3-trifluoromethyl-benzyl)-1H-pyrazol-4-yl]-1,6-dihydro-purin-9-ylmethyl ester (340),
2,2-Dimethyl-propionic acid 2-chloro-6-oxo-1-propyl-8-[1-(3-trifluoromethyl-benzyl)-1H-pyrazol-4-yl]-1,6-dihydro-purin-7-ylmethyl ester (341),
7-Methoxymethyl-1-propyl-8-[1-(3-trifluoromethyl-benzyl)-1 H-pyrazol-4-yl]-1,7-dihydro-purin-6-one (342),
9-Methoxymethyl-1-propyl-8-[1-(3-trifluoromethyl-benzyl)-1H-pyrazol-4-yl]-1,9-dihydro-purin-6-one (343), 2-Chloro-7-methoxymethyl-1-propyl-8-[1-(3-trifluoromethyl-benzyl)-1H-pyrazol-4-y1]-1, 7-dihydro-purin-6-one (344),
Phosphoric acid mono-{6-oxo-1-propyl-8-[1-(3-trifluoromethyl-benzyl)-1H-pyrazol-4-yl]-1,6-dihydro-purin-7-ylmethyl} ester (345),
(2-Dimethylamino-ethyl)-methyl-carbamic acid 6-oxo-1-propyl-8-[1-(3-trifluoromethyl-benzyl)-1 H-pyrazol-4-yl]-1,6-dihydro-purin-7-ylmethyl ester (346),
(1-Ethyl-pyrrolidin-2-ylmethyl)-carbamic acid 6-oxo-1-propyl-8-[1-(3-trifluoromethyl-benzyl)-1 H-pyrazol-4-yl]-1,6-dihydro-purin-7-ylmethyl ester (347),

Nicotinic acid 6-oxo-1-propyl-8-[1-(3-trifluoromethyl-benzyl)-1 H-pyrazol-4-yl]-1,6-dihydro-purin-7-ylmethyl ester (348),

Acetic acid 6-oxo-1-propyl-8-[1-(3-trifluoromethyl-benzyl)-1H-pyrazol-4-yl]-1,6-dihydro-purin-7-ylmethyl ester (349),

Butyric acid 6-oxo-1-propyl-8-[1-(3-trifluoromethyl-benzyl)-1H-pyrazol-4-yl]-1,6-dihydro-purin-7-ylmethyl ester (350),

Butyric acid 2-chloro-6-oxo-1-propyl-8-[1-(3-trifluoromethyl-benzyl)-1 H-pyrazol-4-y1]-1,6-dihydro-purin-7-ylmethyl ester (351),

Nicotinic acid 2-chloro-6-oxo-1-propyl-8-[1-(3-trifluoromethyl-benzyl)-1H-pyrazol-4-yl]-1,6-dihydro-purin-7-ylmethyl ester (352), (2-Dimethylamino-ethyl)-methyl-carbamic acid 2-chloro-6-oxo-1-propyl-8-[1-(3-trifluoromethyl-benzyl)-1 H-pyrazol-4-yl]-1,6-dihydro-purin-7-ylmethyl ester (353),
(2-Dimethylamino-ethyl)-methyl-carbamic acid 2-cyano-6-oxo-1-propyl-8-[1-(3-trifluoromethyl-benzyl)-1 H-pyrazol-4-yl]-1,6-dihydro-purin-7-ylmethyl ester (354),
Butyric acid 2-cyano-6-oxo-1-propyl-8-[1-(3-trifluoromethyl-benzyl)-1H-pyrazol-4-yl]-1,6-dihydro-purin-7-ylmethyl ester (355), 2,2-Dimethyl-propionic acid 2-cyano-6-oxo-1-propyl-8-[1-(3-trifluoromethyl-benzyl)-1H-pyrazol-4-yl]-1,6-dihydro-purin-7-ylmethyl ester (356), Nicotinic acid 2-cyano-6-oxo-1-propyl-8-[1-(3-trifluoromethyl-benzyl)-1H-pyrazol-4-yl]-1,6-dihydro-purin-7-ylmethyl ester (357),
4-Methyl-piperazine-1-carboxylic acid 2-cyano-6-oxo-1-propyl-8-[1-(3-trifluoromethyl-benzyl)-1 H-pyrazol-4-yl]-1,6-dihydro-purin-7-ylmethyl ester (358),
1-{6-Oxo-7-phosphonooxymethyl-1-propyl-8-[1-(3-trifluoromethyl-benzyl)-1H-pyrazol-4-yl]-6,7-dihydro-1H-purin-2-yl}-pyrrolidine-2-carboxylic acid (359),
1-{7-(2 ,2-Dimethyl-propionyloxymethyl)-6-oxo-1-propyl-8-[1-(3-trifluoromethyl-benzyl)-1H-pyrazol-4-yl]-6,7-dihydro-1H-purin-2-yl}-pyrrolidine-2-carboxylic acid (360),
2,2-Dimethyl-propionic acid 2-cyclopropyl-6-oxo-1-propyl-8-[1-(3-trifluoromethyl-benzyl)-1 H-pyrazol-4-yl]-1,6-dihydro-purin-7-ylmethyl ester (361),
Phosphoric acid mono-{2-cyclopropyl-6-oxo-1-propyl-8-[1-(3-trifluoromethyl-benzyl)-1 H-pyrazol-4-yl]-1,6-dihydro-purin-7-ylmethyl} ester (362),
Phosphoric acid mono-{2-chloro-6-oxo-1-propyl-8-[1-(6-trifluoromethyl-pyridin-3-ylmethyl)-1H-pyrazol-4-yl]-1,6-dihydro-purin-7-ylmethyl} ester (363),
Phosphoric acid mono-{6-oxo-1-propyl-8-[1-(6-trifluoromethyl-pyridin-3-ylmethyl)-1 H-pyrazol-4-yl]-1,6-dihydro-purin-7-ylmethyl} ester (364),
Benzoic acid 2-cyano-6-oxo-1-propyl-8-[1-(3-trifluoromethyl-benzyl)-1H-pyrazol-4-yl]-1,6-dihydro-purin-7-ylmethyl ester (365),
7-Methoxymethyl-6-oxo-1-propyl-8-[1-(3-trifluoromethyl-benzyl)-1H-pyrazol-4-yl]-6, 7-dihydro-1H-purine-2-carbonitrile (366),
Acetic acid 2-cyano-6-oxo-1-propyl-8-[1-(3-trifluoromethyl-benzyl)-1H-pyrazol-4-yl]-1,6-dihydro-purin-7-ylmethyl ester (367),
(S)-Pyrrolidine-1,2-dicarboxylic acid 1-benzyl ester 2-{2-cyano-6-oxo-1-propyl-8-[1-(3-trifluoromethyl-benzyl)-1H-pyrazol-4-yl]-1,6-dihydro-purin- 7-ylmethyl} ester (368),
Butyric acid 2-cyano-6-oxo-1-propyl-8-[1-(3-trifluoromethyl-benzyl)-1H-pyrazol-4-yl]-1,6-dihydro-purin-9-ylmethyl ester (369), Butyric acid 2- cyclopropyl-6-oxo-1-propyl-8-[1-(3-trifluoromethyl-benzyl)-1H-pyrazol-4-yl]-1,6-dihydro-purin-9-ylmethyl ester (370),
Butyric acid 2-chloro-6-oxo-1-propyl-8-[1-(3-trifluoromethyl-benzyl)-1 H-pyrazol-4-yl]-1,6-dihydro-purin-9-ylmethyl ester (371),
Butyric acid 6-oxo-1-propyl-8-[1-(3-trifluoromethyl-benzyl)-1H-pyrazol-4-yl]-1,6-dihydro-purin-9-ylmethyl ester (372),
Phosphoric acid mono-{2-fluoro-6-oxo-1-propyl-8-[1-(3-trifluoromethyl-benzyl)-1H-pyrazol-4-y1]-1,6-dihydro-purin-9-ylmethyl} ester (373),
Phosphoric acid mono-{6-oxo-1-propyl-8-[1-(3-trifluoromethyl-benzyl)-1H-pyrazol-4-yl]-1,6-dihydro-purin-9-ylmethyl} ester (374), or
Phosphoric acid mono-{2-cyclopropyl-6-oxo-1-propyl-8-[1-(3-trifluoromethyl-benzyl)-1H-pyrazol-4-yl]-1,6-dihydro-purin-9-ylmethyl} ester (375).

In an embodiment of the present disclosure there is provided a pharmaceutical composition comprising compounds selected from the compound of Formula I, compound of

Formula II, compound of Formula III, or compound of Formula IV for the manufacture of a medicament for the treatment of a condition or disorder ameliorated by inhibition of the A2 A/A2B receptor further comprising a therapeutically effective amount of at least one pharmaceutically acceptable excipient.

### EXAMPLE 1

### Synthesis of the Compound of Formula

The compounds of Formula I were synthesized as per the procedures mentioned in W02012038980 which is incorporated herein by reference. Pharmaceutically acceptable salts of the compounds may be obtained as per procedures reported in literature.

### EXAMPLE 2

### Synthesis of the Compound of Formula

The compounds of Formula II were synthesized as per the procedures mentioned in WO2010103547 which is incorporated herein by reference. Pharmaceutically acceptable salts of the compounds may be obtained as per procedures reported in literature.

### EXAMPLE 3

### Synthesis of the Compound of Formula III and Formula IV

The compounds of Formula III or IV were synthesized as per the procedures mentioned in WO2012035548 which is incorporated herein by reference. Pharmaceutically acceptable salts of the compounds may be obtained as per procedures reported in literature.

The compounds of the disclosure may be prepared by a variety of methods, including standard synthetic chemistry. Any previously defined variable will continue to have the previously defined meaning unless otherwise indicated. Illustrative general synthetic methods are set out in the schemes and can be readily adapted to prepare other compounds of the disclosure.

### EXAMPLE 4

### Biological Assay

Tumor suppression activity of Adenosine A2A (Compound 31) and A2B (Compound 169) Antagonists in xenograft models of cancer

General Protocol: 6-8 weeks old BALB/c mice were acclimated and on Day 1 of the study, the mice were injected with 50 pL of medium containing 5 X104 4T1 cells (breast cancer) or 5 X 105 CT26 cells (colon cancer). On day 8 or 9, the mice were segregated into different groups and treated orally with either vehicle (1% Tween-80+0.5% Carboxymethylcellulose in water) or test compound [(Compound 32 5-Amino-3-[2-[4-[2-fluoro-4-(2-methoxyethoxy)phenyl]piperazin-1-yl]ethyl]-8-(2-furyl)-1-methyl-[1,2,4]triazolo[5,1-f]purin-2-one (32) & Phosphoric acid mono-{2-cyano-6-oxo-1-propyl-8-[1-(3-trifluoromethyl-benzyl)-1H-pyrazol-4-yl]-1,6-dihydro-purin-7-ylmethyl} ester (335)] in vehicle. The treatment was BID for 22 days (colon cancer) or 28 days (breast cancer). At the end of the study, the tumor volume was measured as (length X breadth)/2. Data were presented as % reduction compared to the tumor volume in vehicle treated animals.

4T1 Breast Cancer Model

| | % decrease in Tumor growth |
|---|---|
| Treatment Groups (n = 12) | vs. vehicle On Day 28 |
| Compound 32, 1 mg/kg, *PO, **BID | 48.30 |
| Compound 335, 3 mg/kg, *PO, **BID | 46.40 |

CT26 Colon Cancer Model

| | % decrease in Tumor growth |
|---|---|
| Treatment Groups (n = 12) | vs. vehicle On Day 22 |
| Compound 32, 1 mg/kg, *PO, **BID | 54.40 |
| Compound 335, 3 mg/kg, *PO, **BID | 23.90 |

*PO = per os, i.e., by mouth;
**BID = bis in die, i.e., twice a day

Although the subject matter has been described in considerable detail with reference to certain preferred embodiments thereof, other embodiments are possible. As such, the spirit and scope of the appended claims should not be limited to the description of the preferred embodiment contained therein.

It is claimed:

1. A method for treating a cancer comprising: administering to a patient in need thereof a pharmaceutical composition comprising a compound of Formula I or a pharmaceutically acceptable salt, analog, tautomeric form, stereoisomer, geometrical isomer, polymorph, hydrate, solvate, or prodrug thereof 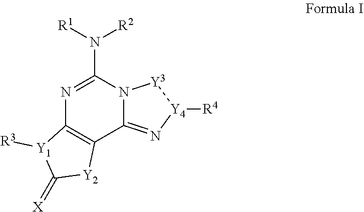
wherein
represents
a single bond or a double bond;
X is selected from O, S or NRa; Y1 is N; Y2 is NR5;
Y3 is selected from N, CH, CH2, C(═O), or C(═S);
Y4 is selected from N, C, or CH; R1 and R2 are independently selected from hydrogen or alkyl;
R3 is -A-Z-B-Q; wherein, A is absent or is a group selected from alkylene, alkenylene, or alkynylene; wherein one or more methylene group is optionally replaced by hetero atoms or groups such as —O—, —S(O)p-, —N(Ra)—, or —C(O); alkylene, alkenylene and alkynylene is optionally substituted with —(CRdRe)nOR7, (CRdRe)nCOOR7, —(CRdRe)nNR8R9, cyano, halogen, haloalkyl, perhaloalkyl, alkoxyalkoxy, alkyl, or cycloalkyl;
Z is absent or is selected from a cycloalkyl or a heterocyclyl; wherein cycloalkyl and heterocyclyl are unsubstituted or substituted independently with 1, 2, or 3 substituents independently selected from alkyl, alkenyl, alkynyl, acyl, —(CRdRe)nOR7, (CRdRe)nCOOR7, —(CRdRe)nNR8R9, aminocarbonyl, alkoxycarbonylamino, halogen, haloalkyl, perhaloalkyl, azido, cyano, keto, thiocarbonyl, —SO3H, aminocarbonylamino, nitro, —S(O)2NRaRa, —NRbS(O)2Rb or —S(O)pRc;
B is absent or is a group selected from alkylene, alkenylene or alkynylene; wherein one or more methylene groups is optionally replaced by hetero atoms or groups such as —O—, —S(O)p-, —N(Ra)—, or —C(O); alkylene, alkenylene and alkynylene is optionally substituted with hydroxy, amino, aminoalkyl, cyano, halogen, haloalkyl, perhaloalkyl, carboxy, carboxyalkyl, alkoxy, hydroxyalkyl, alkoxyalkyl, alkoxyalkoxy or alkyl;
Q is selected from hydrogen, alkyl, cycloalkyl, cycloalkylalkyl, aryl, arylalkyl, heterocyclyl, heterocyclylalkyl, heteroaryl or heteroarylalkyl; wherein alkyl, cycloalkyl, cycloalkylalkyl, aryl, arylalkyl, heterocyclyl, heterocyclylalkyl, heteroaryl and heteroarylalkyl are unsubstituted or substituted independently with 1, 2, or 3 substituents independently selected from alkyl, alkenyl, alkynyl, halogen, haloalkyl, perhaloalkyl, azido, cyano, nitro, keto, thiocarbonyl, cyanoalkyl, cyanoalkylcarbonyl, —(CRdRe)nOR7, —(CRdRc)nC(O)R7, —(CRdRc)nSR7, —(CRdRc)nCOOR7,—(CRdRc)nNR8R9, —(CRdRc)nC(O)NR8R9, —(CRdRc)nNR8C(O)OR7,—(CRdRc)nNR8C(O)NR8R9, —NRbS(O)2Rh, —S(O)pRc, —SO3H, —S(O)2NRaRa, cycloalkyl, cycloalkenyl, cycloalkylalkyl aryl, arylalkyl, heterocyclyl, heterocyclylalkyl, heteroaryl or heteroarylalkyl; wherein cach substituent is unsubstituted or substituted with 1, 2, or 3 substituents independently selected from alkyl, carboxy, carboxyalkyl, aminocarbonyl, hydroxy, alkoxy, halogen, haloalkyl, perhaloalkyl, haloalkoxy, perhaloalkoxy, amino, substituted amino, cyano or —S(O)pRc, R4 is selected from the group consisting of hydrogen, alkyl, alkenyl, alkynyl, haloalkyl, hydroxyalkyl, carboxyalkyl, cycloalkyl, cycloalkylalkyl, aryl, arylalkyl, heterocyclyl, heterocyclylalkyl, heteroaryl and heteroarylalkyl; wherein alkyl, alkenyl, alkynyl, cycloalkyl, cycloalkylalkyl, arylalkyl, aryl, heteroaryl, heteroarylalkyl, heterocyclyl, and heterocyclylalkyl are unsubstituted or substituted independently with up to four substituents independently selected from alkyl, alkenyl, alkynyl, acyl, —(CRdRe)nOR7, (CRdRc)nCOOR7,—(CRdRc)nNR8R9, aminocarbonyl, alkoxycarbonylamino, aminocarbonylamino, azido, cyano, halogen, haloalkyl, perhaloalkyl, keto, nitro, —S(O)2NRbRb, —NRbS(O)2Rb or —S(O)pRc, thiocarbonyl, —SO3H, cycloalkyl, cycloalkenyl, aryl, heteroaryl or heterocyclyl;
R5 and R6 are independently selected from the group consisting of hydrogen, hydroxy, —(CRdRc)nOR7, (CRdRc)nCOOR7, —(CRdRc)nNR8R9, cyanoalkyl, haloalkyl, alkoxyalkoxyalkyl, alkyl, alkenyl, alkynyl, cycloalkyl, cycloalkylalkyl, aryl, arylalkyl, heterocyclyl, heterocyclylalkyl, heteroaryl and heteroarylalkyl;
R7 is selected from hydrogen, alkyl, halogen, haloalkyl, —(CRdRe)nOR7, —(CRdRc)nCOOR7,—(CReRc)nC(O)R7, carbonylamino, cycloalkyl, cycloalkylalkyl, aryl, arylalkyl, heteroaryl, heteroarylalkyl, heterocyclyl or heterocyclylalkyl;
R8 and R9 are independently selected from the group consisting of hydrogen, alkyl, haloalkyl, —(CRdRc)nOR7, —(CRdRe)nC(O)R7, aryl, arylalkyl, heteroaryl, heteroarylalkyl, cycloalkyl, cycloalkylalkyl, heterocyclyl and heterocyclylalkyl, or R8 and R9 taken together form a monocyclic or a bicyclic ring system which is saturated or partially unsaturated and optionally have additional heteroatoms selected from 0, N or S, said ring system is further optionally substituted with I to 4 substituents independently selected from halo, alkyl, alkenyl, alkynyl, nitro, cyano, —(CRdRc)nOR7, —(CRdRc)nSR7, —(CRdRc)nNR8R9, oxo, alkylsulfonyl, —(CRdRc)nCOOR7, —(CRdRc)nC(O)NR8R9, cycloalkyl, cycloalkylalkyl, aryl, arylalkyl, heterocyclyl, heterocyclylalkyl, heteroaryl, or heteroarylalkyl;
Ra is selected from hydrogen or alkyl; Rb each is independently selected from the group consisting of hydrogen, alkyl, acyl, carboxyalkyl, carbonylamino, cycloalkyl, cycloalkylalkyl, aryl, arylalkyl, heteroaryl, heteroarylalkyl, heterocyclyl and heterocyclylalkyl; Re is selected from alkyl, cycloalkyl, aryl, heterocyclyl or heteroaryl; Rd and Re are independently selected from the group consisting of hydrogen, —OR7, halogen, haloalkyl, perhaloalkyl and alkyl;
n is 0, 1, 2, 3 or 4, and
p is 0, 1 or 2.

2. The method of claim 1, wherein the cancer is selected from the group consisting of prostate cancer, rectal cancer, renal cancer, ovarian cancer, endometrial cancer, thyroid cancer, pancreatic cancer, breast cancer, colon cancer, bladder cancer, brain cancer, glial cancer, melanoma cancer, pineal gland cancer, head and neck cancer, and lung cancer.

3. The method of claim 2, wherein the compound of Formula I is selected from the group consisting of:
5-Amino-8-(2-furyl)-3-[2-[4-[4-(2-methoxyethoxy)phesnyl]piperazin-1-yl]ethyl]-1 methyl-[1,2,4]triazolo[5, 1-f]purin-2-one (1),
5-Amino-8-(2-furyl)-3-(2-hydroxyethyl)-1-methyl-[1,2,4]triazolo[5, 1-f]purin-2-one (2),
5-Amino-3-[2-[4-(2,4-difluorophenyl)piperazin-1-yl]ethyl]-8-(2-furyl)-1-methyl-[],2,4]triazolo[5, 1-f]purin-2-one (3),
5-Amino-8-(2-furyl)-3-[2-[4-(4-methoxyphenyl)piperazin-1-yl]ethyl]-1-methyl-[1,2,4]triazolo[5, 1-f]purin-2-one (4),
5-Amino-8-(2-furyl)-1-methyl-3-(2-morpholinoethyl)-[1,2,4]triazolo[5, 1-f]purin-2-one (5),
5-Amino-3-[2-[4-(2,4-difluorophenyl)-1-piperidyl]ethyl]-8-(2-furyl)-1-methyl[1,2,4]triazolo[5, 1-f]purin-2-one (6),
5-Amino-8-(2-furyl)-1-methyl-3-[2-[4-(5-methyl-2-pyridyl)piperazin-1-yl]ethyl] [1,2,4]triazolo[5, 1-f]purin-2-one (7),
5-Amino-8-(2-furyl)-1-methyl-3-[2-[4-(p-tolyl)piperazin-1-yl]ethyl]-[1,2,4]triazolo[5, 1-f]purin-2-one (8),
5-Amino-8-(2-furyl)-1-methyl-3-[2-[4-(3-methyl-2-oxo-butyl)piperazin-1-yl]ethyl] [1,2,4]triazolo[5, 1-f]purin-2-one (9),
5-Amino-3-[2-[4-(2-fluoro-4-methoxy-phenyl)piperazin-1-yl]ethyl]-8-(2-furyl)-1-methyl[1,2,4]triazolo[5, 1-f]purin-2-one (10),
5-Amino-8-(2-furyl)-3-[2-[4-[4-(2-methoxy-1,1-dimethyl-ethoxy)phenyl]piperazin-1 yl]ethyl]-1-methyl-[1,2,4]triazolo[5, 1-f]purin-2-one (11),
5-Amino-8-(2-furyl)-3-[2-[4-(6-methoxy-3-pyridyl)piperazin-1-yl]ethyl]-1-methyl-[1,2,4]triazolo[5, 1-f]purin-2-one (12),
5-Amino-3-[2-[4-[3-fluoro-4-(2-methoxyethoxy)phenyl]piperazin-1-yl]ethyl]-8-(2-furyl)-1-methyl-[1,2,4]triazolo[5, 1-f]purin-2-one (13),
5-Amino-8-(2-furyl)-3-[2-[4-[4-(1-hydroxy-1-methyl-ethyl)phenyl]piperazin-1-yl]ethyl]-1-methyl-[1,2,4]triazolo[5, 1-f]purin-2-one (14),
5-Amino-3-[2-[4-(4-fluorophenyl)-4-hydroxy-1-piperidyl]ethyl]-8-(2-furyl)-1-methyl[],2,4]triazolo[5, 1-f]purin-2-one (15),
5-Amino-8-(2-furyl)-3-[2-[4-[4-(2-methoxy-2-methyl-propoxy)phenyl]piperazin-1 yl]ethyl]-1-methyl-[1,2,4]triazolo[5, 1-f]purin-2-one (16),
5-Amino-3-[2-[4-[4-(cyclopropoxy)phenyl]piperazin-1-yl]ethyl]-8-(2-furyl)-1-methyl [1,2,4]triazolo[5, 1-f]purin-2-one (17),
5-Amino-3-[2-[4-(4-fluorophenyl)-3,6-dihydro-2H-pyridin-1-yl]ethyl]-8-(2-furyl)-1 methyl-[1,2,4]triazolo[5, 1-f]purin-2-one (18),
5-Amino-8-(2-furyl)-3-[2-[4-hydroxy-4-(4-methoxyphenyl)-1-piperidyl]ethyl]-1-methyl [1,2,4]triazolo[5, 1-f]purin-2-one (19),
5-Amino-3-[2-[4-[3,5-difluoro-4-(2-methoxyethoxy)phenyl]piperazin-1-yl]ethyl]-8-(2-furyl)-1-methyl-[1,2,4]triazolo[5, 1-f]purin-2-one (20),
5-Amino-3-[2-[4-[2,5-difluoro-4-(2-methoxyethoxy)phenyl]piperazin-1-yl]ethyl]-8-(2-furyl)-1-methyl-[1,2,4]triazolo[5, 1-f]purin-2-one (21),
5-Amino-3-[2-[4-(2,2-difluoro-1,3-benzodioxol-5-yl)piperazin-1-yl]ethyl]-8-(2-furyl)-1 methyl-[],2,4]triazolo[5, 1-f]purin-2-one (22),
5-Amino-8-(2-furyl)-3-[2-[4-[4-(2-methoxyethoxy)phenyl]-3,3-dimethyl-piperazin-1 yl]ethyl]-1-methyl-[1,2,4]triazolo[5, 1-f]purin-2-one (23),
5-Amino-3-[2-(4-butylpiperazin-1-yl)ethyl]-8-(2-furyl)-1-methyl-[1,2,4]triazolo[5, 1-f]purin-2-one (24),
5-Amino-8-(2-furyl)-3-[2-(4-hydroxy-4-methyl-1-piperidyl)ethyl]-I-methyl [1,2,4]triazolo[5, 1-f]purin-2-one (25),
5-Amino-3-[2-[4-[4-[2-(cyclopropoxy)ethoxy]phenyl]piperazin-1-yl]ethyl]-8-(2-furyl)-1 methyl-[1,2,4]triazolo[5, 1-f]purin-2-one (26),
5-Amino-8-(2-furyl)-3-[2-[4-[(4-methoxyphenyl)methyl]piperazin-1-yl]ethyl]-1-methyl [1,2,4]triazolo[5, 1-f]purin-2-one (27),
5-Amino-8-(2-furyl)-3-[2-[4-[[4-(2-methoxyethoxy)phenyl]methyl]piperazin-1-yl]ethyl]-1 methyl-[1,2,4]triazolo[5, 1-f]purin-2-one (28),
5-Amino-8-(2-furyl)-3-[(4-methoxyphenyl)methyl]-I-methyl-[1,2,4]triazolo[5, 1-f]purin-2-one (29),
5-Amino-8-(2-furyl)-3-[2-[4-(4-methoxyphenyl)piperazin-1-yl]ethyl]-1-methyl [1,2,4]triazolo[5, 1-f]purin-2-one (30),
5-Amino-8-(2-furyl)-3-[2-[4-[3-(2-methoxyethoxy)phenyl]piperazin-1-yl]ethyl]-1-methyl [1,2,4]triazolo[5, 1-f]purin-2-one (31),
5-Amino-3-[2-[4-[2-fluoro-4-(2-methoxyethoxy)phenyl]piperazin-1-yl]ethyl]-8-(2-furyl)-1 methyl-[1,2,4]triazolo[5, 1-f]purin-2-one (32),
4-[4-[2-[5-Amino-8-(2-furyl)-1-methyl-2-oxo-[1,2,4]triazolo[5, 1-f]purin-3yl]ethyl]piperazin-1-yl]benzonitrile (33), 4-[4-[2-[5-Amino-8-(2-furyl)-1-methyl-2-oxo-[1,2,4]triazolo[5, 1-f]purin-3-yl]ethyl]piperazin-1-y1]-2-fluoro-benzonitrile (34),
5-Amino-8-(2-furyl)-1-methyl-3-[2-[4-[4-(trifluoromethyl)phenyl]piperazin-1-yl]ethyl] [1,2,4]triazolo[5, 1-f]purin-2-one (35),
5-Amino-8-(2-furyl)-1-methyl-3-[2-[4-[4-(trifluoromethyl)thiazol-2-yl]piperazin-1-yl]ethyl] [1,2,4]triazolo[5, 1-f]purin-2-one (36),
5-Amino-3-[2-[4-(cyclopropylmethyl)piperazin-1-yl]ethyl]-8-(2-furyl)-1-methyl [1,2,4]triazolo[5, 1-f]purin-2-one (37),
5-Amino-3-[2-(4-ethylpiperazin-1-yl)ethyl]-8-(2-furyl)-1-methyl-[1,2,4]triazolo[5, 1 f]purin-2-one (38),
4-[2-[5-Amino-8-(2-furyl)-1-methyl-2-oxo-[1,2,4]triazolo[5, 1-f]purin-3-yl]ethyl]-N,N dimethyl-piperazine-1-sulfonamide (39),
5-Amino-8-(2-furyl)-1-methyl-3-[2-[4-(4-tetrahydrofuran-3-yloxyphenyl)piperazin-1 yl]ethyl]-[1,2,4]triazolo[5, 1-f]purin-2-one (40),
5-Amino-8-(2-furyl)-1-methyl-3-[2-[4-(4-tetrahydropyran-4-yloxyphenyl)piperazin-1 yl]ethyl]-[1,2,4]triazolo[5, 1-f]purin-2-one (41),
5-Amino-8-(2-furyl)-1-methyl-3-[2-[4-[4-(tetrahydrofuran-2-ylmethoxy)phenyl]piperazin-1-yl]ethyl]-[1,2,4]triazolo[5, 1-f]purin-2-one (42),
5-Amino-8-(2-furyl)-1-methyl-3-[2-(3-methyl-7,8-dihydro-5H-1,6-naphthyridin-6-yl)ethyl]-[1,2,4]triazolo[5, 1-f]purin-2-one (43),
5-Amino-3-[2-(6,7-dihydro-4H-thieno[3,2-c]pyridin-5-yl)ethyl]-8-(2-furyl)-1-methyl [1,2,4]triazolo[5, 1-f]purin-2-one (44),
5-Amino-8-(2-furyl)-3-[2-[4-[4-(2-methoxyethoxy)phenyl]piperazin-1-yl]propyl]-1 methyl-[1,2,4]triazolo[5, 1-f]purin-2-one (45),
5-Amino-3-[2-[3-(4-fluorophenyl)-2,5-dihydropyrrol-1-yl]ethyl]-8-(2-furyl)-1-methyl [1,2,4]triazolo[5, 1-f]purin-2-one (46),
5-Amino-3-[2-[4-[4-(2-methoxyethoxy)phenyl]piperazin-1-yl]ethyl]-1-methyl-8-(5-methyl-2-furyl)-[1,2,4]triazolo[5, 1-f]purin-2-one (47),
5-Amino-8-(5-cyclopropyl-2-furyl)-3-[2-[4-[4-(2-methoxyethoxy)phenyl]piperazin-1-yl]ethyl]-1-methyl-[1,2,4]triazolo[5, 1-f]purin-2-one (48),
5-Amino-3-[2-(2,4-difluoroanilino)ethyl]-8-(2-furyl)-1-methyl-[1,2,4]triazolo[5, 1 f]purin-2-one (49),
5-Amino-3-[3-[4-(4-fluorophenyl)piperazin-1-yl]propyl]-8-(2-furyl)-1-methyl [1,2,4]triazolo[5, 1-f]purin-2-one (50),
5-Amino-8-(2-furyl)-3-[3-[4-[4-(2-methoxyethoxy)phenyl]piperazin-1-yl]propyl]-1 methyl-[1,2,4]triazolo[5, 1-f]purin-2-one (51),
5-Amino-8-(2-furyl)-3-[2-[4-(4-methoxyphenyl)-3,6-dihydro-2H-pyridin-1-yl]ethyl]-1 methyl-[1,2,4]triazolo[5, 1-f]purin-2-one (52),
5-Amino-8-(2-furyl)-3-[2-[4-[4-(2-methoxy-1,1-dimethyl-ethyl)phenyl]piperazin-1 yl]ethyl]-1-methyl-[1,2,4]triazolo[5,1-f]purin-2-one (53),
5-Amino-8-(2-furyl)-1-methyl-3-(2-piperazin-1-ylethyl)-[1,2,4]triazolo[5,1-f]purin-2-one (54),
5-Amino-8-(2-furyl)-3-[2-[4-(IH-indole-2-carbonyl)piperazin-1-yl]ethyl]-1-methyl [1,2,4]triazolo[5, 1-f]purin-2-one (55),
5-Amino-8-(2-furyl)-3-[2-(4-isopropoxyphenyl)ethyl]-I-methyl-[1,2,4]triazolo[5, 1 f]purin-2-one (56),
5-Amino-8-(2-furyl)-1-methyl-3-[2-[4-[(2S)-pyrrolidine-2-carbonyl]piperazin-1 yl]ethyl]-[1,2,4]triazolo[5, 1-f]purin-2-one (57),
5-Amino-8-(2-furyl)-3-[2-(4-methoxyphenyl)ethyl]-I-methyl-[1,2,4]triazolo[5, 1-f]purin-2-one (58),
5-amino-3-[2-[4-[4-(difluoromethoxy)phenyl]piperazin-1-yl]ethyl]-8-(2-furyl)-1-methyl [1,2,4]triazolo[5, 1-f]purin-2-one (59),
5-Amino-8-(2-furyl)-1-methyl-3-[2-[4-[3-(5-methyl-1,3,4-oxadiazol-2-yl)phenyl ]piperazin-1-yl]ethyl]-[1,2,4]triazolo[5, 1-f]purin-2-one (60),
5-Amino-3-[2-[4-[2-fluoro-4-(5-methyl-1,2,4-oxadiazol-3-yl)phenyl]piperazin-1-yl]ethyl]-8-(2-furyl)-1-methyl-[1,2,4]triazolo[5, 1-f]purin-2-one (61),
5-Amino-3-[2-[4-(6-fluoro-2-methyl-1,3-benzoxazol-5-yl)piperazin-1-yl]ethyl]-8-(2-furyl)-1-methyl-[1,2,4]triazolo[5, 1-f]purin-2-one (62),
5-Amino-3-[2-[4-(cyclopropanecarbonyl)piperazin-1-yl]ethyl]-8-(2-furyl)-1-methyl [1,2,4]triazolo[5, 1-f]purin-2-one (63),
5-Amino-3-[2-[4-(2-cyclopropylacetyl)piperazin-1-yl]ethyl]-8-(2-furyl)-1-methyl [1,2,4]triazolo[5, 1-f]purin-2-one (64),
5-Amino-8-(2-furyl)-3-[2-[4-[4-(2-hydroxyethoxy)phenyl]piperazin-1-yl]ethyl]-lmethyl-[1,2,4]triazolo[5, 1-f]purin-2-one (65),
5-Amino-8-(2-furyl)-3-[2-[4-(4-hydroxyphenyl)piperazin-1-yl]ethyl]-1-methyl [1,2,4]triazolo[5, 1-f]purin-2-one (66),
5-Amino-1-(cyclopropylmethyl)-3-[2-[4-(4-ethoxyphenyl)piperazin-1-yl]ethyl]-8-(2-furyl)-[1,2,4]triazolo[5, 1-f]purin-2-one (67),
5-Amino-1-(cyclopropylmethyl)-3-[2-[4-(4-fluorophenyl)piperazin-1-yl]ethyl]-8-(2-furyl)-[1,2,4]triazolo[5, 1-f]purin-2-one (68),
5-Amino-1-(cyclopropylmethyl)-3-[2-[4-(2,4-difluorophenyl)piperazin-1-yl]ethyl]-8-(2-furyl)-[1,2,4]triazolo[5, 1-f]purin-2-one (69),
5-Amino-1-(cyclopropylmethyl)-8-(2-furyl)-3-[2-[4-[4-(2-methoxyethoxy)phenyl]piperazin-1-yl]ethyl]-[1,2,4]triazolo[5, 1-f]purin-2-one (70),
5-Amino-1-(cyclopropylmethyl)-3-[2-(4-fluorophenoxy)ethyl]-8-(2-furyl)-[1,2,4]triazolo[5, 1-f]purin-2-one (71),
5-Amino-8-(2-furyl)-1-methyl-3-[2-[2-oxo-5-(trifluoromethyl)-1-pyridyl]ethyl] [1,2,4]triazolo[5, 1-f]purin-2-one (72),
5-Amino-3-[2-[4-(2,4-difluorophenyl)pyrazol-1-yl]ethyl]-1-ethyl-8-(2-furyl) [1,2,4]triazolo[5, 1-f]purin-2-one (73),
1-[2-[5-Amino-1-(cyclopropylmethyl)-8-(2-furyl)-2-oxo-[1,2,4]triazolo[5, 1-f]purin-3-ylethyl]pyrazole-4-carboxylic acid (74),
1-[2-[5-Amino-8-(2-furyl)-1-methyl-2-oxo-[1,2,4]triazolo[5, 1-f]purin-3-yl]ethyl]pyrazole-4-carboxylic acid (75),
1-[2-[5-amino-1-(cyclopropylmethyl)-8-(2-furyl)-2-oxo-[1,2,4]triazolo[5, 1-f]purin-3-yl]ethyl]-N-cyclopropyl-pyrazole-4-carboxamide (76),
1-[2-[5-amino-1-(cyclopropylmethyl)-8-(2-furyl)-2-oxo-[1,2,4]triazolo[5, 1-f]purin-3-yl]ethyl]-N,N-diethyl-pyrazole-4-carboxamide (77),
1-[2-[5-Amino-1-(cyclopropylmethyl)-8-(2-furyl)-2-oxo-[1,2,4]triazolo[5, 1-f]purin-3-yl]ethyl]-N-cyclopropyl-5-methyl-pyrazole-3- carboxamide (78),
2-[2-[5-Amino-1-(cyclopropylmethyl)-8-(2-furyl)-2-oxo-[1,2,4]triazolo[5, 1-f]purin-3-yl]ethyl]-N-cyclopropyl-5-methyl-pyrazole-3- carboxamide (79),
1-[2-[5-Amino-8-(2-furyl)-1-methyl-2-oxo-[1,2,4]triazolo[5, 1-f]purin-3-yl]ethyl]-N methyl-pyrazole-3-carboxamide (80),
1-[2-[5-Amino-8-(2-furyl)-1-methyl-2-oxo-[1,2,4]triazolo[5, 1-f]purin-3-yl]ethyl]-N,N diethyl-pyrazole-4-carboxamide (81),
1-[2-[5-amino-8-(2-furyl)-1-methyl-2-oxo-[1,2,4]triazolo[5, 1-f]purin-3-yl]ethyl]pyrazole-4-carboxamide (82),
5-Amino-8-(2-furyl)-3-[2-[4-[(3R)-3-hydroxypyrrolidine-1-carbonyl]pyrazol-1-yl]ethyl]-1-methyl-[1,2,4]triazolo[5, 1-f]purin-2-one (83),
1-[2-[5-Amino-8-(2-furyl)-1-methyl-2-oxo-[1,2,4]triazolo[5, 1-f]purin-3-yl]ethyl]-N methyl-pyrazole-4-carboxamide (84),
1-[2-[5-Amino-8-(2-furyl)-1-methyl-2-oxo-[1,2,4]triazolo[5, 1-f]purin-3-yl]ethyl]-N cyclopropyl-pyrazole-3-carboxamide (85),
1-[2-[5-Amino-8-(2-furyl)-1-methyl-2-oxo-[1,2,4]triazolo[5, 1-f]purin-3-yl]ethyl]-N cyclopropyl-pyrazole-4-carboxamide (86),
5-Amino-8-(2-furyl)-3-[2-[4-(3-hydroxyazetidine-1-carbonyl)pyrazol-1-yl]ethyl ]-1-methyl-[1,2,4]triazolo[5, 1-f]purin-2-one (87),
5-Amino-1-ethyl-8-(2-furyl)-3-[2-[4-[4-(2-methoxyethoxy)phenyl]piperazin-1-yl]ethyl] [1,2,4]triazolo[5, 1-f]purin-2-one (88),
5-Amino-3-[2-[4-(2,4-difluorophenyl)piperazin-1-yl]ethyl ]-1-ethyl-8-(2-furyl) [1,2,4]triazolo[5, 1-f]purin-2-one (89),
5-Amino-1-ethyl-3-{2-[4-(4-fluoro-phenyl)-piperidin-1-yl]-ethyl}-8-furan-2-yl-1,3-dihydro-[1,2,4]triazolo[5, 1-f]purin-2-one (90),
5-Amino-1-ethyl-8-(2-furyl)-3-[2-(3-methyl-7,8-dihydro-5H-1,6-naphthyridin-6-yl)ethyl]-[1,2,4]triazolo[5, 1-f]purin-2-one (91),
5-Amino-8-(2-furyl)-3-[2-[4-[4-(2-methoxyethoxy)phenyl]piperazin-1-yl]ethyl]-1-(2,2,2-trifluoroethyl)-[1,2,4]triazolo[5, 1-f]purin-2-one (92),
5-Amino-3-{2-[4-(2,4-difluoro-phenyl)-piperazin-1-yl]-ethyl}-8-furan-2-yl-1-(2,2,2-trifluoro-ethyl)-1,3-dihydro-[1,2,4]triazolo[5, 1-f]purin-2-one (93),
5-Amino-8-(2-furyl)-3-[2-[4-[4-(2-methoxyethoxy)phenyl]piperazin-1-yl]ethyl]-1-(2-methoxyethyl)-[1,2,4]triazolo[5, 1-f]purin-2-one (94), 5-amino-3-[2-[4-(4-fluorophenyl)piperazin-1-yl]ethyl]-8-(2-furyl)-1-(2-methoxyethyl) [1,2,4]triazolo[5, 1-f]purin-2-one (95),
5-Amino-3-[2-[4-(4-fluorophenyl)piperazin-1-yl]ethyl]-8-(2-furyl)-1-(2-hydroxyethyl) [1,2,4]triazolo[5, 1-f]purin-2-one one (96),
5-Amino-1-cyclopropyl-8-(2-furyl)-3-[2-[4-[4-(2-methoxyethoxy)phenyl]piperazin-1-yl]ethyl]-[1,2,4]triazolo [5, 1-f]purin-2-one (97),
5-Amino-8-(2-furyl)-3-[2-[4-[4-(2-methoxyethoxy)phenyl]piperazin-1-yl]ethyl]-1-(2,2,2-trifluoroethyl)-[1,2,4]triazolo[5, 1-f]purin-2-one (98),
5-Amino-3-[2-[4-[4-(2-methoxyethoxy)phenyl]piperazin-1-yl]ethyl]-1-methyl-8-thiazol-2-yl-[1,2,4]triazolo[5, 1-f]purin-2-one (99),
5-Amino-3-[2-[4-[3-fluoro-4-(2-methoxyethoxy)phenyl]piperazin-1-yl]ethyl]-1-methyl-8-thiazol-2-yl-[1,2,4]triazolo[5, 1-f]purin-2-one (100),
5-Amino-3-[2-[4-(2-cyclopropylacetyl)piperazin-1-yl]ethyl]-1-methyl-8-thiazol-2-yl [1,2,4]triazolo[5, 1-f]purin-2-one (101),
5-Amino-3-[2-[4-(4-methoxyphenyl)piperazin-1-yl]ethyl]-1-methyl-8-thiazol-2-yl [1,2,4]triazolo[5, 1-f]purin-2-one (102),
5-Amino-1-methyl-3-[2-[4-(p-tolyl)piperazin-1-yl]ethyl]-8-thiazol-2-yl [1,2,4]triazolo[5, 1-f]purin-2-one (103),
5-Amino-1-methyl-3-[2-(3-methyl-7,8-dihydro—SH-1,6-naphthyridin-6-yl)ethyl]-8-thiazol-2-yl-[1,2,4]triazolo[5, 1-f]purin-2-one (104),
4-[4-[2-(
5-Amino-1-methyl-2-oxo-8-thiazol-2-y]-[1,2,4]triazolo[5, 1-f]purin-3-yl)ethyl ]piperazin-1-yl]benzonitrile (105),
5-Amino-1-methyl-3-[2-[4-[3-(5-methyl-1,3,4-oxadiazol-2-yl)phenyl]piperazin-1-yl]ethyl]-8-thiazol-2-yl-[1,2,4]triazolo[5, 1-f]purin-2-one (106),
5-amino-3-[2-[4-[4-(1-hydroxy-1-methyl-ethyl)phenyl ]piperazin-1-yl]ethyl]-1-methyl-8-thiazol-2-yl-[1,2,4]triazolo[5, 1-f]purin-2-one (107),
5-Amino-3-[2-[4-[4-(2-methoxyethoxy)phenyl]piperazin-1-yl]ethyl]-1-methyl-8-(2-pyridyl)-[],2,4]triazolo[5, 1-f]purin-2-one (108),
5-Amino-3-[2-[4-(2,4-difluorophenyl)piperazin-1-yl]ethyl]-1-methyl-8-(2-pyridyl) [1,2,4]triazolo[5, 1-f]purin-2-one (109), 4-[4-[2-[5-Amino-1-methyl-2-oxo-8-(2-pyridyl)-[1,2,4]triazolo[5, 1-f]purin-3-yl]ethyl]piperazin-1-yl]benzonitrile (110),
5-Amino-3-[2-[4-[4-(1-hydroxy-1-methyl-ethyl)phenyl]piperazin-1-yl]ethyl]-1-methyl-8-(2-pyridyl)-[1,2,4]triazolo[5, 1-f]purin-2-one (111),
5-Amino-3-[2-[4-[4-(2-methoxyethoxy)phenyl ]piperazin-1-yl]ethyl]-1-methyl-8-pyrazin-2-yl-[1,2,4]triazolo[5, 1-f]purin-2-one (112),
5-Amino-3-[2-[4-(2,4-difluorophenyl)piperazin-1-yl]ethyl]-1-methyl-8-pyrazin-2-yl [1,2,4]triazolo[5, 1-f]purin-2-one (113),
5-Amino-3-[2-[4-[2-fluoro-4-(2-methoxyethoxy)phenyl]piperazin-1-yl]ethyl]-1-methyl-8-pyrazin-2-yl-[1,2,4]triazolo[5, 1-f]purin-2-one (114),
5-Amino-8-(2-furyl)-3-[[1-(4-methoxyphenyl)pyrrolidin-3-yl]methyl ]-1-methyl[],2,4]triazolo[5, 1-f]purin-2-one (115),
5-Amino-8-(2-furyl)-3-[1-[4-(2-methoxyethoxy)phenyl]pyrrolidin-3-yl]methyl]-1 methyl-[1,2,4]triazolo[5, 1-f]purin-2-onehyl}-1-methyl-1,3-dihydro-[1,2,4]triazolo[5,1 i]purin-2-one (116), 5-amino-8-(2-furyl)-3-[2-[4-[4-(2-methoxyethoxy)phenyl]piperazin-1-yl]ethyl]-1-methyl [1,2,4]triazolo[5, 1-f]purine-2-thione (117),
8-(2-furyl)-3-[2-[4-[4-(2-methoxyethoxy)phenyl]piperazin-1-yl]ethyl]-1-methyl-5-(methylamino)-[1,2,4]triazolo[5,1-f]purin-2-one (118),
5-Amino-3-{2-[4-(4-fluoro-phenyl)-piperazin-1-yl]-ethyl}-8-isothiazol-5-yl-1-methyl-1,3-dihydro-[1,2,4]triazolo[5, 1-i]purin-2-one (119),
5-Amino-8-isothiazol-5-yl-3-(2-{4-[4-(2-methoxy-ethoxy)-phenyl]-piperazin-1-yl} ethyl)-1-methyl-1,3-dihydro-[1,2,4]triazolo[5, 1-i]purin-2-one (120),
5-Amino-3-[2-[4-[2-fluoro-4-(2-methoxyethoxy)phenyl]piperazin-1-yl]ethyl]-8-isothiazol-5-yl-1-methyl-[1,2,4]triazolo[5, 1-f]purin-2-one (121),
5-Amino-8-isoxazol-5-yl-3-[2-[4-[4-(2-methoxyethoxy)phenyl]piperazin-1-yl]ethyl]-lmethyl-[1,2,4]triazolo[5, 1-f]purin-2-one (122),
5-Amino-3-[2-[4-[4-(2-methoxyethoxy)phenyl ]piperazin-1-yl]ethyl]-1-methyl-8-oxazol-2-yl-[1,2,4]triazolo[5, 1-f]purin-2-one (123),
5-Amino-3-{2-[4-(4-methoxy-phenyl)-piperazin-1-yl]-ethyl}-1-methyl-8-prop-1-ynyl-1,3-dihydro-[1,2,4]triazolo[5, 1-i]purin-2-one (124),
5-Amino-3-(2-{4-[4-(2-methoxy-ethoxy)-phenyl]-piperazin-1-yl}-ethyl)-1-methyl-8-prop-1-ynyl-1,3-dihydro-[1,2,4]triazolo[5,1-i]purin-2-one (125),
5-Amino-3-{2-[4-(4-fluoro-benzoyl)-piperazin-1-yl]-ethyl}-8-furan-2-yl-1-methyl-1,3-dihydro-[1,2,4]triazolo[5, 1-i]purin-2-one (126),
5-Amino-3-(2-dimethylamino-ethyl)-8-furan-2-yl-1-methyl-1,3-dihydro-[1,2,4]triazolo[5, 1-i]purin-2-one (127),
5-Amino-8-furan-2-yl-3-[3-(4-methoxy-phenyl)-propyl]-1-methyl-1,3-dihydro [1,2,4]triazolo[5, 1-i]purin-2-one (128),
5-Amino-8-furan-2-yl-1-methyl-3-(2-pyrazol-1-yl-ethyl)-1,3-dihydro-[1,2,4]triazolo[5, I i]purin-2-one (129),
5-Amino-8-furan-2-yl-3-(2-{4-[4-(2-methoxy-ethoxy)-phenyl]-pyrazol-1-yl}-ethyl)-1 methyl-1,3-dihydro-[1,2,4]triazolo[5, 1-i]purin-2-one (130),
5-Amino-8-furan-2-yl-3-{2-[3-(4-methoxy-phenyl)-pyrrol-1-yl]-ethyl}-1-methyl-1,3-dihydro-[1,2,4]triazolo[5, 1-i]purin-2-one (131),
5-Amino-8-furan-2-yl-3-{2-[4-(4-methoxy-phenyl)-imidazol-1-yl]-ethyl}-1-methyl-1,3-dihydro-[1,2,4]triazolo[5, 1-i]purin-2-one (132),
5-Amino-8-furan-2-yl-3-{2-[4-(4-methoxy-phenyl)-[1,2,3]triazol-1-yl ]-ethyl}-1-methyl-1,3-dihydro-[1,2,4]triazolo[5, 1-i]purin-2-one (133),
5-Amino-3-[2-(1,3-dihydro-isoindol-2-yl)-ethyl]-8-furan-2-yl-1-methyl-1,3-dihydro[1,2,4]triazolo[5, 1-i]purin-2-one (134),
5-Amino-8-furan-2-yl-1-methyl-3-(2-piperidin-1-yl-ethyl)-1,3-dihydro-[1,2,4]triazolo[5, 1-i]purin-2-one (135),
5-Amino-8-furan-2-yl-1-methyl-3-(2-pyrrolidin-1-yl-ethyl)-1,3-dihydro-[1,2,4]triazolo[5, 1-i]purin-2-one (136),
5-Amino-8-furan-2-yl-1-methyl-3-[2-(3-methyl-7,8-dihydro-5H-[1,6]naphthyridin-6-yl) ethyl]-1,3-dihydro-[1,2,4]triazolo[5, 1-i]purin-2-one (137),
5-Amino-8-furan-2-yl-3-{2-[4-(2-methoxy-ethoxy)-phenoxy]-ethyl}-1-methyl-1,3-dihydro-[1,2,4]triazolo[5,1-i]purin-2-one (138),
5-Amino-8-furan-2-yl-3-{2-[4-(2-methoxy-ethoxy)-phenylamino]-ethyl}-1-methyl-1,3-dihydro-[1,2,4]triazolo[5, 1-i]purin-2-one (139),
5-Amino-8-furan-2-yl-1-methyl-3-[2-(pyridin-2-yloxy)-ethyl]-1,3-dihydro-[1,2,4]triazolo[5, 1-i]purin-2-one (140),
5-Amino-1-ethyl-3-{2-[4-(4-fluoro-phenyl)-piperazin-1-yl]-ethyl}-8-isothiazol-5-yl-1,3-dihydro-[1,2,4]triazolo[5,1-i]purin-2-one (141),
5-Amino-1-ethyl-8-isothiazol-5-yl-3-(2-{4-[4-(2-methoxy-ethoxy)-phenyl]-piperazin-1-yl}-ethyl)-1,3-dihydro-[1,2,4]triazolo[5, 1-i]purin-2-one (142),
5-Amino-1-ethyl-8-furan-2-yl-3-(2-piperidin-1-yl-ethyl)-1,3-dihydro-[1,2,4]triazolo[5, 1-i]purin-2-one (143),
5-Amino-1-ethyl-8-furan-2-yl-3-[2-(3-methyl-7,8-dihydro-5H-[1,6]naphthyridin-6-yl)-ethyl]-1,3-dihydro-[1,2,4]triazolo[5,1-i]purin-2-one (144),
5-Amino-3-[2-(2,4-difluoro-phenoxy)-ethyl]-1-ethyl-8-furan-2-yl-1,3-dihydro-[1,2,4]triazolo[5, 1-i]purin-2-one (145),
5-Amino-3-[2-(2,4-difluoro-phenylamino)-ethyl]-1-ethyl-8-furan-2-yl-1,3-dihydro [1,2,4]triazolo[5, 1-i]purin-2-one (146),
5-Amino-1-cyclopropylmethyl-3-[2-(2,4-difluoro-phenylamino)-ethyl]-8-furan-2-yl-1,3-dihydro-[1,2,4]triazolo[5, 1-i]purin-2-one (147),
5-Amino-1-cyclopropylmethyl-3-[2-(2,4-difluoro-phenoxy)-ethyl]-8-furan-2-yl-1,3-dihydro-[],2,4]triazolo[5, 1-i]purin-2-one (148),
5-Amino-1-cyclopropylmethyl-3-{2-[4-(4-fluoro-phenyl)-piperidin-1-yl]-ethyl}-8-furan-2-yl-1,3-dihydro-[1,2,4]triazolo[5, 1-i]purin-2-one (149),
5-Amino-3-{2-[4-(4-fluoro-phenyl)-piperidin-1-yl]-ethyl}-8-furan-2-yl-1-(2,2,2-trifluoro-ethyl)-1,3-dihydro-[1,2,4]triazolo[5,1-i]purin-2-one (150),
5-Amino-8-furan-2-yl-3-{2-[4-(4-methoxy-phenyl)-piperazin-1-yl]-ethyl}-1-(2,2,2-trifluoro-ethyl)-1,3-dihydro-[1,2,4]triazolo[5,1-i]purin-2-one (151),
5-Amino-3-[2-(4-cyclopropylmethyl-piperazin-1-yl)-ethyl]-8-isothiazol-5-yl-1-(2,2,2-trifluoro-ethyl)-1,3-dihydro-[1,2,4]triazolo[5,1-i]purin-2-one (152), (
5-Amino-8-isothiazol-5-yl-3-{2-[4-(4-methoxy-phenyl)-piperazin-1-yl]-ethyl}-2-oxo-2,3-dihydro-[1,2,4]triazolo[5,1-i]purin-1-yl)-acetonitrile (153),
[5-Amino-3-{2-[4-(2,4-difluoro-phenyl)-piperazin-1-yl]-ethyl}-8-(3-fluoro-phenyl)-2-oxo-2,3-dihydro-[1,2,4]triazolo[5, 1-i]purin-1-yl ]-acetonitrile (154),
[5-Amino-8-furan-2-yl-3-(2-{4-[4-(2-methoxy-ethoxy)-phenyl]-piperazin-1-yl} ethyl)-2-oxo-2,3-dihydro-[1,2,4]triazolo[5,1-i]purin-1-yl ]-acetonitrile (155),
5-Amino-3-(2-{4-[4-(2-methoxy-ethoxy)-phenyl]-piperazin-1-yl}-ethyl)-1-methyl-8-phenyl-1,3-dihydro-[1,2,4]triazolo[5, 1-i]purin-2-one (156),
3-[5-Amino-3-(2-{4-[4-(2-methoxy-ethoxy)-phenyl]-piperazin-1-yl}-ethyl)-1-methyl-2-oxo-2,3-dihydro-1H-[1,2,4]triazolo[5,1- i]purin-8-yl ]-benzonitrile (157),
3-[5-Amino-3-(2-{4-[4-(2-methoxy-ethoxy)-phenyl]-piperazin-1-yl}-ethyl)-1 methyl-2-oxo-2,3-dihydro-1H-[1,2,4]triazolo[5,1- i]purin-8-yl ]-benzonitrile (158),
5-Amino-8-furan-2-yl-1-methyl-3-vinyl-1,3-dihydro-[1,2,4]triazolo[5,1-i]purin-2-one (159)
5-Amino-3-[3-(4-fluoro-phenyl)-prop-2-ynyl]-8-furan-2-yl-1-methyl-1,3-dihydro [1,2,4]triazolo[5, 1-i]purin-2-one (160),
5-Amino-8-furan-2-yl-1-methyl-3-[4-(4-methyl-piperazin-1-yl)-but-2-ynyl]-1,3-dihydro-[1,2,4]triazolo[5, 1-i]purin-2-one (161),
5-Amino-8-furan-2-yl-1-isopropyl-3-(2-{4-[4-(2-methoxy-ethoxy)-phenyl] piperazin-1-yl}-ethyl)-1,3-dihydro-[1,2,4]triazolo[5, 1-i]purin-2-one (162),
5-Amino-2-benzyl-7-(2-{4-[4-(2-methoxy-ethoxy)-phenyl]-piperazin-1-yl} ethyl)-9-methyl-7,9-dihydro-2H-[1,2,4]triazolo[3, 4-i]purine-3,8-dione (163),
5-Amino-2-benzyl-9-methyl-7-(2-morpholin-4-yl-ethyl)-7,9-dihydro-2H-[1,2,4]triazolo[3,4-i]purine-3,8-dione (164),
5-Amino-2-(3-chloro-benzyl)-7-[2-(4-isopropyl-piperazin-1-yl)-ethyl]-9-methyl-7,9-dihydro-2H-[1,2,4]triazolo[3, 4-i]purine-3,8-dione (165),
5-Amino-2-cyclopropylmethyl-9-methyl-7-(2-morpholin-4-yl-ethyl)-7,9-dihydro-2H-[1,2,4]triazolo[3,4-i]purine-3,8-dione (166),
5-Amino-2-cyclopropylmethyl-7-(2,4-difluoro-benzyl)-9-methyl-7,9-dihydro-2H [1,2,4]triazolo[3,4-i]purine-3,8-dione (167), 4-Amino-2-furan-2-yl-6-(2-{4-[4-(2-methoxy-ethoxy)-phenyl ]-piperazin-1-yl} ethyl)-6H-8-oxa-1,3,3a,5,6-pentaaza-as-indacen-7-one (168), and
4-Amino-2-furan-2-yl-6-(2-{4-[4-(2-methoxy-ethoxy)-phenyl ]-piperazin-1-yl} ethyl)-8,8-dimethyl-6,8-dihydro-1,3,3a,5,6-pentaaza-as-indacen-7-one (169).

4. A pharmaceutical composition comprising compound of Formula II and its pharmaceutically acceptable salts, analogs, tautomeric forms, stereoisomers, geometrical isomers, polymorphs, hydrates, solvates, metabolites, and prodrugs thereof 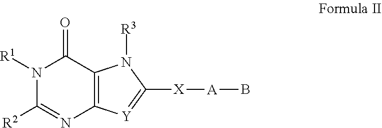
wherein,
Y is selected from N or CR; R is selected from H, hydroxy, alkoxy, alkyl, or aryl; R1 is selected from a group consisting of alkyl, alkenyl and alkynyl, wherein one or more methylene groups are optionally replaced by hetero atoms or groupselected from —O—, —S(O)p-, —N(Ra)—, or —C(O) provided that the heteroatom is not adjacent to N in the ring; p is selected from 0, 1 or 2; wherein alkyl, alkenyl and alkynyl are unsubstituted or substituted independently with alkoxy, acyl, acylamino, acyloxy, amino, monoalkylamino, dialkylamino, aminocarbonyl, alkoxycarbonylamino, azido, cyano, halogen, haloalkyl, hydroxy, hydroxyalkyl, keto, thiocarbonyl, carboxy, alkylcarboxy, carboxyalkyl, —SO3H, aminocarbonylamino, hydroxyamino, alkoxyamino, nitro, —S(O)2NRaRa, —NRaS(O)2Ra, or —S(O)pRa;
R2 is selected from a group consisting of hydrogen, halogen, cyano, nitro, carboxy, acyl, aminocarbonyl, alkyl, alkenyl, alkynyl, hydroxyalkyl, carboxyalkyl, haloalkyl, haloalkyloxy, alkoxy, —NRbRb, —S(O)pRb, cycloalkyl, cycloalkylalkyl, cycloalkyloxy, aryl, arylalkyl, aryloxy, heterocyclyl, heterocyclylalkyl, heterocyclyloxy, heteroaryl, heteroarylalkyl and heteroaryloxy;
wherein alkyl, alkenyl, alkynyl, alkoxy, carboxyalkyl, cycloalkyl, cycloalkylalkyl, cycloalkyloxy, aryl, arylalkyl, aryloxy, heterocyclyl, heterocyclylalkyl, heterocyclyloxy, heteroaryl, heteroarylalkyl, heteroaryloxy and Rb are unsubstituted or substituted independently with alkyl, alkenyl, alkynyl, alkoxy, acyl, acylamino, acyloxy, nitro, ammo, monoalkylamino, dialkylamino, hydroxyamino, alkoxyamino, aminocarbonylamino, azido, cyano, halogen, hydroxy, hydroxyalkyl, keto, thiocarbonyl, carboxy, alkylcarboxy, carboxyalkyl, —SO3H, arylamino, cycloalkylamino, heteroarylamino, heterocyclylamino, aminocarbonyl, alkoxycarbonylamino, cycloalkyl, cycloalkyloxy, cycloalkenyl, aryl, aryloxy, heteroaryl, heteroaryloxy, heterocyclyl, heterocyclyloxy, —S(O)2NRcRc, —NRCS(O)2Rc or —S(O)pRd;
wherein cach substituent is unsubstituted or substituted with 1, 2, or 3 substituents independently selected from alkyl, carboxy, carboxyalkyl, aminocarbonyl, hydroxy, alkoxy, halogen, haloalkyl, haloalkoxy, amino, substituted amino, cyano or —S(O)pRd;
R3 is selected from a group consisting of hydrogen, alkyl, alkenyl, alkynyl, alkoxyalkyl, cycloalkyl, cycloalkylalkyl, aryl, arylalkyl, heteroaryl and heteroarylalkyl; wherein alkyl, alkenyl, alkynyl, alkoxyalkyl, cycloalkyl, cycloalkylalkyl, aryl, arylalkyl, heteroaryl, and heteroarylalkyl are unsubstituted or substituted independently with alkyl, alkenyl, alkynyl, alkoxy, cycloalkyl, cycloalkenyl, acyl, acylamino, acyloxy, amino, monoalkylamino, dialkylamino, arylamino, cycloalkylamino, heteroarylamino, heterocyclylamino, aminocarbonyl, alkoxycarbonylamino, azido, cyano, halogen, hydroxy, hydroxyalkyl, keto, thiocarbonyl, carboxy, alkylcarboxy, carboxyalkyl, carboxyalkyloxy, alkylcarboxyalkyloxy, —SO3H, aryl, aryloxy, cycloalkyloxy, heteroaryl, aminocarbonylamino, heteroaryloxy, heterocyclyl, heterocyclyloxy, hydroxyamino, alkoxyamino, nitro, S(O)2NRcRc, —NRCS(O)2Rc or —S(O)pRd; wherein cach substituent is unsubstituted or substituted with 1, 2, or 3 substituents independently selected from alkyl, carboxy, carboxyalkyl, aminocarbonyl, hydroxyd, alkoxy, halogen, haloalkyl, haloalkoxy, amino, substituted amino, cyano or —S(O)pRd;
X is either an optionally substituted arylene or an optionally substituted heteroarylene;
A is selected from a bond, (C1—C6)alkylene, (C2—C6)alkenylene or (C2—C6)alkynylene group, wherein 1 to 4 methylene groups are optionally replaced by groups independently selected from O, —S(O)p-, —N(Rb)—, or —C(O)—; wherein alkylene, alkenylene, and alkynylene are unsubstituted or substituted independently with alkyl, alkenyl, alkynyl, alkoxy, cycloalkyl, cycloalkenyl, acyl, acylamino, acyloxy, ammo, monoalkylamino, dialkylamino, arylamino, cycloalkylamino, heteroarylamino, heterocyclylamino, aminocarbonyl, alkoxycarbonylamino, azido, cyano, halogen, hydroxy, hydroxyalkyl, keto, thiocarbonyl, carboxy, alkylcarboxy, carboxyalkyl, carboxyalkyloxy, alkylcarboxyalkyloxy—SO3H, aryl, aryloxy, cycloalkyloxy, heteroaryl, aminocarbonylamino, heteroaryloxy, heterocyclyl, heterocyclyloxy, hydroxyamino, alkoxyamino, nitro, S(O)2NRcRc, —NRCS(O)2Rc or —S(O)pRd; wherein each substituent is unsubstituted or substituted with 1, 2, or 3 substituents independently selected from alkyl, carboxy, carboxyalkyl, aminocarbonyl, hydroxy, alkoxy, halogen, CF3, amino, substituted amino, cyano or —S(O)pRd;
B is selected from hydrogen, heterocyclyl, cycloalkyl, aryl or heteroaryl; wherein heterocyclyl, cycloalkyl, aryl and heteroaryl are unsubstituted or substituted independently with alkyl, alkenyl, alkynyl, alkoxy, cycloalkyl, cycloalkylalkyl, cycloalkenyl, acyl, acylamino, acyloxy, ammo, monoalkylamino, dialkylamino, arylamino, cycloalkylamino, heteroarylamino, heterocyclylamino, aminocarbonyl, alkoxycarbonylamino, azido, cyano, halogen, hydroxy, hydroxyalkyl, keto, thiocarbonyl, carboxy, alkylcarboxy, carboxyalkyl, carboxyalkyloxy, alkylcarboxyalkyloxy—SO3H, aryl, arylalkyl, aryloxy, cycloalkyloxy, heteroaryl, heteroarylalkyl, aminocarbonylamino, heteroaryloxy, heterocyclyl, heterocyclylalkyl, heterocyclyloxy, hydroxyamino, alkoxyamino, nitro, —S(O)2NRbRb, —NRbS(O)2Rb or —S(O)pRd; wherein cach substituent is unsubstituted or substituted with 1, 2, or 3 substituents independently selected from alkyl, carboxy, carboxyalkyl, aminocarbonyl, hydroxy, alkoxy, alkoxyalkoxy, alkoxyalkyl, halogen, haloalkyl, haloalkoxy, amino, substituted amino, cyano or —S(O)pRd;
Ra is independently selected from hydrogen or alkyl;
Rb is independently selected from the group consisting of hydrogen, alkyl, acyl, carboxyalkyl, carbonylamino, cycloalkyl, cycloalkylalkyl, aryl, arylalkyl, heteroaryl, heteroarylalkyl, heterocyclyl, and heterocyclylalkyl;
Rc is selected from hydrogen, alkyl, aryl, heteroaryl or heterocyclyl;
Rd is selected from alkyl, cycloalkyl, aryl, heterocyclyl or heteroaryl; and
p is 0, 1 or 2,
for the manufacture of a medicament for the treatment of a condition or disorder ameliorated by inhibition of the A2A/A2B receptor.

5. The pharmaceutical composition as claimed in claim 4, wherein the compound of Formula II is selected from the group consisting of:
8-(4-Benzyloxy-phenyl)-1-propyl-1,7-dihydro-purin-6-one (170),
1-Propyl-8-[1-(3-trifluoromethyl-benzyl)-1 H-pyrazol-4-yl]-1,7-dihydro-purin-6-one (171),
8-(1-Benzyl-1H-pyrazol-4-yl)-1-propyl-1,7-dihydro-purin-6-one (170),
2-Chloro-8-[1-(2,3-difluoro-benzyl)-1H-pyrazol-4-yl]-1-propyl-1,7-dihydro-purin-6-one (172),
2-Chloro-8-[1-(3-fluoro-4-trifluoromethyl-benzyl)-1H-pyrazol-4-yl]-1-propyl-1,7-dihydro-purin-6-one (173),
2-Chloro-1-propyl-8-[1-(4-trifluoromethyl-benzyl)-1H-pyrazol-4-yl]-1,7-dihydro-purin-6-one (174),
8-[1-(3-Fluoro-4-trifluoromethyl-benzyl)-1H-pyrazol-4-yl]-1-propyl-1,7-dihydro-purin-6-one (175),
8-[1-(2,3-Difluoro-benzyl)-1H-pyrazol-4-yl]-1-propyl-1,7-dihydro-purin-6-one (176),
1-Propyl-8-[1-(4-trifluoromethyl-benzyl)-1H-pyrazol-4-yl]-1,7-dihydro-purin-6-one (177),
1-Propyl-8-(1H-pyrazol-4-yl)-1,7-dihydro-purin-6-one (178),
2-Chloro-8-[1-(3-fluoro-benzyl)-1H-pyrazol-4-yl]-1-propyl-1,7-dihydro-purin-6-one (179),
8-[1-(2,4-Difluoro-benzyl)-1H-pyrazol-4-yl]-1-propyl-1,7-dihydro-purin-6-one (180),
2-Chloro-1-propyl-8-[1-(3-trifluoromethyl-benzyl)-1H-pyrazol-4-yl]-1,7-dihydro-purin-6-one (181),
8-{4-[3-(4-Fluoro-phenyl)-prop-2-ynyloxy]-phenyl}-1-propyl-1,7-dihydro-purin-6-one (182),
8-{4-[5-Oxo-1-(4-trifluoromethoxy-phenyl)-pyrrolidin-3-ylmethoxy]-phenyl}-1-propyl-1,7-dihydro-purin-6-one (183), 1-Propyl-8-{4-[3-(3-trifluoromethyl-phenyl)-prop-2-ynyloxy]-phenyl}-1,7-dihydro-purin-6-one (184),
8-{4-[5-Oxo-1-(3-trifluoromethyl-phenyl)-pyrrolidin-3-ylmethoxy]-phenyl}-1-propyl-1,7-dihydro-purin-6-one (185),
2-Chloro-8-[1-(2,4-difluoro-benzyl)-1H-pyrazol-4-yl]-1-propyl-1,7-dihydro-purin-6-one (186),
8-[1-(3-Fluoro-benzyl)-1H-pyrazol-4-yl]-1-propyl-1,7-dihydro-purin-6-one (187),
2-Morpholin-4-yl-1-propyl-8-[1-(4-trifluoromethyl-benzyl)-1H-pyrazol-4-yl]-1,7-dihydro-purin-6-one (188),
N-(4-Cyano-phenyl)-2-[4-(6-oxo-1-propyl-6,7-dihydro-1H-purin-8-yl)-phenoxy]-acetamide (189),
[4-(6-Oxo-1-propyl-6,7-dihydro-1H-purin-8-yl)-phenoxy]-acetic acid (190),
8-(1-Benzyl-1H-pyrazol-4-yl)-2-chloro-1-propyl-1,7-dihydro-purin-6-one (191),
8-(4-{2-Oxo-2-[4-(3-trifluoromethyl-phenyl)-piperazin-1-yl]-ethoxy}-phenyl)-1-propyl-1,7-dihydro-purin-6-one (192),
8-(1-Benzyl-1H-pyrazol-4-yl)-1-propyl-2-(4-trifluoromethyl-benzylamino)-1,7-dihydro-purin-6-one (193),
8-(1-Benzyl-1H-pyrazol-4-yl)-1-propyl-2-(3-trifluoromethyl-benzylamino)-1,7-dihydro-purin-6-one (194),
8-(1-Benzyl-1H-pyrazol-4-yl)-2-[2-(4-methoxy-phenyl)-ethylamino]-1-propyl-1,7-dihydro-purin-6-one (195),
8-(1-Benzyl-1H-pyrazol-4-yl)-2-phenethylamino-1-propyl-1,7-dihydro-purin-6-one (196),
8-(1-Benzyl-1H-pyrazol-4-yl)-2-(4-methyl-piperazin-1-yl)-1-propyl-1,7-dihydro-purin-6-one (197),
8-(1-Benzyl-1H-pyrazol-4-yl)-2-piperidin-1-yl-1-propyl-1,7-dihydro-purin-6-one (198),
8-{1-[1-(2,4-Difluoro-phenyl)-5-oxo-pyrrolidin-3-ylmethyl]-1H-pyrazol-4-yl}-1-propyl-1,7-dihydro-purin-6-one (199),
8-[1-(3-Fluoro-4-trifluoromethyl-benzyl)-1H-pyrazol-4-yl]-2-(2-hydroxy-ethylamino)-1-propyl-1,7-dihydro-purin-6-one (200),
2-Amino-1-propyl-8-[1-(3-trifluoromethyl-benzyl)-1H-pyrazol-4-yl]-1,7-dihydro-purin-6-one (201),
8-(1-Benzyl-1H-pyrazol-4-yl)-2-methylamino-1-propyl-1,7-dihydro-purin-6-one (202),
[8-(1-Benzyl-1H-pyrazol-4-yl)-6-oxo-1-propyl-6,7-dihydro-1H-purin-2-ylamino]-acetic acid ethyl ester (203),
8-(1-Benzyl-1H-pyrazol-4-yl)-2-methoxy-1-propyl-1,7-dihydro-purin-6-one (204),
1,2-Dipropyl-8-[1-(3-trifluoromethyl-benzyl)-1H-pyrazol-4-yl]-1,7-dihydro-purin-6-one (205),
1-Propyl-8-[1-(3-trifluoromethyl-benzyl)-1H-pyrazol-4-yl]-2-(4-trifluoromethyl-phenyl)-1,7-dihydro-purin-6-one (206),
1-Propyl-8-[1-(3-trifluoromethyl-benzyl)-1H-pyrazol-4-yl]-2-(3-trifluoromethyl-phenyl)-1,7-dihydro-purin-6-one (207),
2-(3-Fluoro-phenyl)-1-propyl-8-[1-(3-trifluoromethyl-benzyl)-1H-pyrazol-4-yl]-1,7-dihydro-purin-6-one (208),
2-Dimethylamino-1-propyl-8-[1-(3-trifluoromethyl-benzyl)-1H-pyrazol-4-yl]-1,7-dihydro-purin-6-one (209),
8-[1-(3-Fluoro-4-trifluoromethyl-benzyl)-1H-pyrazol-4-yl]-6-oxo-1-propyl-6,7-dihydro-1H-purine-2-carbonitrile (210),
8-[1-(3-Fluoro-4-trifluoromethyl-benzyl)-1H-pyrazol-4-yl]-6-oxo-1-propyl-6,7-dihydro-1H-purine-2-carboxylic acid (211),
8-(4-Benzyloxy-phenyl)-1-propyl-2-(3-trifluoromethyl-phenyl)-1,7-dihydro-purin-6-one (212),
8-{4-[3-(4-Fluoro-phenyl)-prop-2-ynyloxy]-phenyl}-1-propyl-2-(3-trifluoromethyl-phenyl)-1,7-dihydro-purin-6-one (213),
8-(4-Methoxy-phenyl)-1-propyl-2-(3-trifluoromethyl-phenyl)-1,7-dihydro-purin-6-one (214),
2-Ethyl-1-propyl-8-[1-(3-trifluoromethyl-benzyl)-1H-pyrazol-4-yl]-1,7-dihydro-purin-6-one (215),
2-Benzyl-1-propyl-8-[1-(3-trifluoromethyl-benzyl)-1H-pyrazol-4-yl]-1,7-dihydro-purin-6-one (216),
{6-Oxo-1-propyl-8-[1-(3-trifluoromethyl-benzyl)-1H-pyrazol-4-yl]-6,7-dihydro-1H-purin-2-ylamino}-acetic acid (217),
(S)-1-{6-Oxo-1-propyl-8-[1-(3-trifluoromethyl-benzyl)-1H-pyrazol-4-yl]-6,7-dihydro-1H-purin-2-yl}-pyrrolidine-2-carboxylic acid (218),
1-Propyl-2-pyrrolidin-1-yl-8-[1-(3-trifluoromethyl-benzyl)-1H-pyrazol-4-yl]-1,7-dihydro-purin-6-one (219),
2-Methylamino-1-propyl-8-[1-(3-trifluoromethyl-benzyl)-1H-pyrazol-4-yl]-1,7-dihydro-purin-6-one (220),
2-Cyclobutylamino-1-propyl-8-[1-(3-trifluoromethyl-benzyl)-1H-pyrazol-4-yl]-1,7-dihydro-purin-6-one (221),
2-Chloro-8-[1-(3-fluoro-4-trifluoromethyl-benzyl)-1H-pyrazol-4-yl]-7-methyl-1-propyl-1,7-dihydro-purin-6-one (222), 2-Methoxy-1-propyl-8-[1-(3-trifluoromethyl-benzyl)-1H-pyrazol-4-yl]-1,7-dihydro-purin-6-one (223),
6-Oxo-1-propyl-8-[1-(3-trifluoromethyl-benzyl)-1H-pyrazol-4-yl]-6,7-dihydro-1H-purine-2-carbonitrile (224),
2-Cyclopentyloxy-1-propyl-8-[1-(3-trifluoromethyl-benzyl)-1H-pyrazol-4-yl]-1,7-dihydro-purin-6-one (225),
6-Oxo-1-propyl-8-[1-(3-trifluoromethyl-benzyl)-1H-pyrazol-4-yl]-6,7-dihydro-1H-purine-2-carboxylic acid amide (226),
{6-Oxo-1-propyl-8-[1-(3-trifluoromethyl-benzyl)-1H-pyrazol-4-yl]-6,7-dihydro-1H-purin-2-yloxy }-acetic acid ethyl ester (227),
2-Morpholin-4-yl-1-propyl-8-[1-(3-trifluoromethyl-benzyl)-1H-pyrazol-4-yl]-1,7-dihydro-purin-6-one (228),
{6-Oxo-1-propyl-8-[1-(3-trifluoromethyl-benzyl)-1H-pyrazol-4-yl]-6,7-dihydro-1H-purin-2-yloxy }-acetic acid (229),
8-{1-[3-(4-Fluoro-phenyl)-prop-2-ynyl]-1H-pyrazol-4-yl}-1-propyl-2-pyrrolidin-1-yl-1,7-dihydro-purin-6-one (230), (S)-1-{6-Oxo-1-propyl-8-[1-(3-trifluoromethyl-benzyl)-1H-pyrazol-4-yl]-6,7-dihydro-1H-purin-2-yl}-pyrrolidine-2-carboxylic acid amide (231),
1-{6-Oxo-1-propyl-8-[1-(3-trifluoromethyl-benzyl)-1H-pyrazol-4-yl]-6,7-dihydro-1H-purin-2-y1}-piperidine-3-carboxylic acid (232),
1-{6-Oxo-1-propyl-8-[1-(3-trifluoromethyl-benzyl)-1H-pyrazol-4-yl]-6,7-dihydro-1H-purin-2-y1}-piperidine-4-carboxylic acid (233),
(2R,4R)-4-Hydroxy-1-{6-oxo-1-propyl-8-[1-(3-trifluoromethyl-benzyl)-1H-pyrazol-4-yl]-6,7-dihydro-1H-purin-2-yl}-pyrrolidine-2-carboxylic acid (234),
2-(2,3-Dihydroxy-propylamino)-1-propyl-8-[1-(3-trifluoromethyl-benzyl)-1H-pyrazol-4-yl]-1,7-dihydro-purin-6-one (235),
2-(2-Methoxy-ethylamino)-1-propyl-8-[1-(3-trifluoromethyl-benzyl)-1H-pyrazol-4-yl]-1,7-dihydro-purin-6-one (236),
2-(4-Hydroxy-piperidin-1-yl)-1-propyl-8-[1-(3-trifluoromethyl-benzyl)-1H-pyrazol-4-yl]-1,7-dihydro-purin-6-one (237),
2-(3-Hydroxy-piperidin-1-yl)-1-propyl-8-[1-(3-trifluoromethyl-benzyl)-1H-pyrazol-4-yl]-1,7-dihydro-purin-6-one (238),
2-{6-Oxo-1-propyl-8-[1-(3-trifluoromethyl-benzyl)-1H-pyrazol-4-yl]-6,7-dihydro-1H-purin-2-ylamino }-ethanesulfonic acid (239),
2-(3-Hydroxymethyl-piperidin-1-yl)-1-propyl-8-[1-(3-trifluoromethyl-benzyl)-1H-pyrazol-4-yl]-1,7-dihydro-purin-6-one (240),
(Methyl-{6-oxo-1-propyl-8-[1-(3-trifluoromethyl-benzyl)-1H-pyrazol-4-yl]-6,7-dihydro-1H-purin-2-yl}-amino)-acetic acid (241),
2-(2-Hydroxy-ethylamino)-1-propyl-8-[1-(3-trifluoromethyl-benzyl)-1H-pyrazol-4-yl]-1,7-dihydro-purin-6-one (242),
2-(4-Hydroxymethyl-piperidin-1-yl)-1-propyl-8-[1-(3-trifluoromethyl-benzyl)-1H-pyrazol-4-yl]-1,7-dihydro-purin-6-one (243),
2-(4-Hydroxymethyl-piperidin-1-yl)-1-propyl-8-[1-(3-trifluoromethyl-benzyl)-1H-pyrazol-4-yl]-1,7-dihydro-purin-6-one (244),
(S)-3-Methyl-2-{6-oxo-1-propyl-8-[1-(3-trifluoromethyl-benzyl)-1H-pyrazol-4-yl]-6,7-dihydro-1H-purin-2-ylamino}-butyric acid (245),
2-((S)-2-Methoxymethyl-pyrrolidin-1-yl)-1-propyl-8-[1-(3-trifluoromethyl-benzyl)-1H-pyrazol-4-yl]-1,7-dihydro-purin-6-one (246),
2-((S)-2-Hydroxymethyl-pyrrolidin-1-yl)-1-propyl-8-[1-(3-trifluoromethyl-benzyl)-1H-pyrazol-4-yl]-1,7-dihydro-purin-6-one (247),
2-((R)-3-Hydroxy-pyrrolidin-1-yl)-1-propyl-8-[1-(3-trifluoromethyl-benzyl)-1H-pyrazol-4-yl]-1,7-dihydro-purin-6-one (248),
1-Propyl-2-(tetrahydro-pyran-4-ylamino)-8-[1-(3-trifluoromethyl-benzyl)-1H-pyrazol-4-yl]-1,7-dihydro-purin-6-one (249), 2-Fluoro-1-propyl-8-[1-(3-trifluoromethyl-benzyl)-1H-pyrazol-4-yl]-1,7-dihydro-purin-6-one (250),
1-Propyl-2-(2,2,2-trifluoro-ethoxy)-8-[1-(3-trifluoromethyl-benzyl)-1H-pyrazol-4-yl]-1,7-dihydro-purin-6-one (251),
2-(2-Methoxy-ethoxy)-1-propyl-8-[1-(3-trifluoromethyl-benzyl)-1H-pyrazol-4-yl]-1,7-dihydro-purin-6-one (252),
7-Methyl-1-propyl-8-[1-(3-trifluoromethyl-benzyl)-1H-pyrazol-4-yl]-1,7-dihydro-purin-6-one (253),
2-Chloro-1-propyl-8-[6-(3-trifluoromethyl-benzylamino)-pyridin-3-yl]-1,7-dihydro-purin-6-one (254),
2-Chloro-8-[6-(3-fluoro-benzylamino)-pyridin-3-yl]-1-propyl-1,7-dihydro-purin-6-one (255),
1-Propyl-8-[6-(3-trifluoromethyl-benzylamino)-pyridin-3-yl]-1,7-dihydro-purin-6-one (256),
1-Propyl-8-(1-pyridin-3-ylmethyl-1H-pyrazol-4-yl)-1,7-dihydro-purin-6-one (257),
1-Propyl-8-[1-(6-trifluoromethyl-pyridin-3-ylmethyl)-1H-pyrazol-4-yl]-1,7-dihydro-purin-6-one (258),
2-Cyclopropyl-1-propyl-8-[1-(3-trifluoromethyl-benzyl)-1H-pyrazol-4-yl]-1,7-dihydro-purin-6-one (259),
2-Difluoromethoxy-1-propyl-8-[1-(3-trifluoromethyl-benzyl)-1H-pyrazol-4-yl]-1,7-dihydro-purin-6-one (260),
1-Propyl-2-trifluoromethyl-8-[1-(3-trifluoromethyl-benzyl)-1H-pyrazol-4-yl]-1,7-dihydro-purin-6-one (261),
2-Chloro-8-(1-methyl-1H-pyrazol-4-yl)-1-propyl-1,7-dihydro-purin-6-one (262),
8-(1-Methyl-1H-pyrazol-4-yl)-1-propyl-1,7-dihydro-purin-6-one (263),
2-Isobutylamino-8-(1-methyl-1H-pyrazol-4-yl)-1-propyl-1,7-dihydro-purin-6-one (264),
8-(1-Methyl-1H-pyrazol-4-yl)-1-propyl-2-pyrrolidin-1-yl-1,7-dihydro-purin-6-one (265),
2-[2-(4-Methoxy-phenyl)-ethylamino]-8-(1-methyl-1H-pyrazol-4-yl)-1-propyl-1,7-dihydro-purin-6-one (266),
2-(4-Methyl-piperazin-1-yl)-8-(1-methyl-1H-pyrazol-4-yl)-1-propyl-1,7-dihydro-purin-6-one (267),
2-[4-(4-Fluoro-phenyl)-piperazin-1-yl]-8-(1-methyl-1H-pyrazol-4-yl)-1-propyl-1,7-dihydro-purin-6-one (268),
1-[8-(1-Methyl-1H-pyrazol-4-yl)-6-oxo-1-propyl-6,7-dihydro-1H-purin-2-yl]-pyrrolidine-2-carboxylic acid methyl ester (269),
2-Benzyl-8-(1-methyl-1H-pyrazol-4-yl)-1-propyl-1,7-dihydro-purin-6-one (270),
2-(3-Fluoro-phenyl)-8-(1-methyl-1H-pyrazol-4-yl)-1-propyl-1,7-dihydro-purin-6-one (271),
8-(1-Methyl-1H-pyrazol-4-yl)-1-propyl-2-(4-trifluoromethyl-phenyl)-1,7-dihydro-purin-6-one (272),
8-(1-Methyl-1H-pyrazol-4-yl)-2-phenethylamino-1-propyl-1,7-dihydro-purin-6-one (273),
8-(1-Methyl-1H-pyrazol-4-yl)-1-propyl-2-(4-trifluoromethyl-benzylamino)-1,7-dihydro-purin-6-one (274),
2-Cyclopropylamino-8-(1-methyl-1H-pyrazol-4-yl)-1-propyl-1,7-dihydro-purin-6-one (275),
2-(3-Fluoro-phenoxy)-8-(1-methyl-1H-pyrazol-4-yl)-1-propyl-1,7-dihydro-purin-6-one (276),
2-(4-Methoxy-phenylamino)-8-(1-methyl-1H-pyrazol-4-yl)-1-propyl-1,7-dihydro-purin-6-one (277),
7-Benzyl-2-chloro-8-(1-methyl-1H-pyrazol-4-yl)-1-propyl-1,7-dihydro-purin-6-one (278),
9-Benzyl-2-chloro-8-(1-methyl-1H-pyrazol-4-yl)-1-propyl-1,9-dihydro-purin-6-one (279),
2-Amino-7-benzyl-8-(1-methyl-1H-pyrazol-4-yl)-1-propyl-1,7-dihydro-purin-6-one (280),
2-Chloro-8-furan-2-yl-1-propyl-1,7-dihydro-purin-6-one (281),
2-Amino-8-[1-(4-fluoro-benzyl)-1H-imidazo[1,2-b]pyrazol-7-yl]-1-propyl-1,7-dihydro-purin-6-one (282),
2-Chloro-8-[1-(4-fluoro-benzyl)-1H-imidazo[1,2-b]pyrazol-7-yl]-1-propyl-1,7-dihydro-purin-6-one (283),
2-Amino-8-(1-methyl-1H-pyrazol-4-yl)-1-propyl-1,7-dihydro-purin-6-one (284),
2-Amino-7-methyl-8-(1-methyl-1H-pyrazol-4-yl)-1-propyl-1,7-dihydro-purin-6-one (285),
2-Amino-9-methyl-8-(1-methyl-1H-pyrazol-4-yl)-1-propyl-1,9-dihydro-purin-6-one (286),
7-Methyl-8-(1-methyl-1H-pyrazol-4-yl)-1-propyl-1,7-dihydro-purin-6-one (287),
9-Methyl-8-(1-methyl-1H-pyrazol-4-yl)-1-propyl-1,9-dihydro-purin-6-one (288),
2-Amino-8-furan-2-yl-1-propyl-1,7-dihydro-purin-6-one (289),
2-Chloro-8-furan-2-yl-7-methyl-1-propyl-1,7-dihydro-purin-6-one (290),
8-(1-Methyl-1H-pyrazol-4-yl)-1-propyl-2-(3-trifluoromethyl-benzylamino)-1,7-dihydro-purin-6-one (291),
2-Furan-2-yl-8-(1-methyl-1H-pyrazol-4-yl)-1-propyl-1,7-dihydro-purin-6-one (292),
8-(1-Benzyl-1H-pyrazol-4-yl)-2-furan-2-yl-1-propyl-1,7-dihydro-purin-6-one (293),
2-Chloro-8-(6-chloro-pyridin-3-yl)-1-propyl-1,7-dihydro-purin-6-one (294),
2-Difluoromethyl-1-propyl-8-[1-(3-trifluoromethyl-benzyl)-1H-pyrazol-4-yl]-1,7-dihydro-purin-6-one (295),
2-Fluoromethyl-1-propyl-8-[1-(3-trifluoromethyl-benzyl)-1H-pyrazol-4-yl]-1,7-dihydro-purin-6-one (296),
2-Fluoromethyl-8-{1-[3-(3-methoxy-phenyl)-prop-2-ynyl]-1H-pyrazol-4-yl}-1-propyl-1,7-dihydro-purin-6-one (297),
2-Difluoromethyl-8-{1-[2-oxo-2-(4-m-tolyl-piperazin-1-yl)-ethyl]-1H-pyrazol-4-yl}-1-propyl-1, 7-dihydro-purin-6-one (298),
3-Fluoro-N-methyl-N-[5-(6-oxo-1-propyl-6,7-dihydro-1H-purin-8-yl)-pyridin-2-yl]-benzamide (299),
N-[5-(2-Difluoromethyl-6-oxo-1-propyl-6,7-dihydro-1H-purin-8-yl)-pyridin-2-yl]-3-methoxy-N-methyl-benzamide (300),
N-[5-(2-Difluoromethyl-6-oxo-1-propyl-6,7-dihydro-1H-purin-8-yl)-pyridin-2-yl]-3-methoxy-benzenesulfonamide (301),
2-Fluoromethyl-1-propyl-8-[1-(5-trifluoromethyl-pyridin-3-ylmethyl)-1H-pyrazol-4-yl]-1,7-dihydro-purin-6-one (302),
2-Fluoromethyl-1-propyl-8-[1-(2-trifluoromethyl-pyridin-4-ylmethyl)-1H-pyrazol-4-yl]-1,7-dihydro-purin-6-one (303),
2-Fluoromethyl-8-[3-(3-methoxy-phenoxy)-isoxazol-5-yl]-1-propyl-1,7-dihydro-purin-6-one (304),
2-Difluoromethyl-8-{3-[3-(3-fluoro-phenyl)-prop-2-ynyloxy]-isoxazol-5-yl}-1-propyl-1,7-dihydro-purin-6-one (305),
2-Fluoromethyl-1-(2-hydroxy-ethyl)-8-[3-(3-methoxy-phenoxy)-isoxazol-5-yl]-1,7-dihydro-purin-6-one (306),
2-Difluoromethyl-1-ethyl-8-{3-[3-(3-fluoro-phenyl)-prop-2-ynyloxy]-isoxazol-5-yl}-1,7-dihydro-purin-6-one (307),
2-Difluoromethyl-1-ethyl-8-(1-{2-[4-(3-methoxy-phenyl)-piperazin-1-yl]-2-oxo-ethyl}-1H-pyrazol-4-yl)-1,7-dihydro-purin-6-one (308),
1-Ethyl-8-(1-{2-[4-(3-methoxy-phenyl)-piperazin-1-yl]-2-oxo-ethyl}-1H-pyrazol-4-yl)-6-oxo-6,7-dihydro-1H-purine-2-carbonitrile (309),
N-[5-(2-Cyano-6-oxo-1-propyl-6,7-dihydro-1H-purin-8-yl)-pyridin-2-yl]-3-methoxy-benzenesulfonamide (310),
N-{5-[2-Cyano-1-(2-hydroxy-ethyl)-6-oxo-6,7-dihydro-1H-purin-8-yl]-pyridin-2-yl}-3-methoxy-benzenesulfonamide (311),
2-Difluoromethyl-1-ethyl-8-{4-[3-(3-methoxy-phenyl)-prop-2-ynyloxy]-phenyl}-1,7-dihydro-purin-6-one (312),
2-Difluoromethyl-1-ethyl-8-{4-[1-(3-fluoro-phenyl)-5-oxo-pyrrolidin-3-ylmethoxy]-phenyl}-1,7-dihydro-purin-6-one (313),
2-Difluoromethyl-8-[5-(3-methoxy-phenoxy)-1-methyl-1H-pyrazol-3-yl]-1-propyl-1,7-dihydro-purin-6-one (314),
2-Difluoromethyl-8-{5-[1-(3-methoxy-phenyl)-piperidin-4-yloxy]-1-methyl-1H-pyrazol-3-yl}-1-propyl-1,7-dihydro-purin-6-one (315),
2-Fluoromethyl-8-{3-[1-(3-fluoro-phenyl)-piperidin-4-yloxy]-isoxazol-5-yl}-1-propyl-1,7-dihydro-purin-6-one (316), 1-Ethyl-8-{6-[1-(3-fluoro-phenyl)-5-oxo-pyrrolidin-3-ylmethoxy]-pyridin-3-yl}-6-oxo-6,7-dihydro-1H-purine-2-carbonitrile (317),
1-Ethyl-8-{6-[1-(3-methoxy-phenyl)-pyrrolidin-3-yloxy]-pyridin-3-yl}-6-oxo-6,7-dihydro-1H-purine-2-carbonitrile (318),
3-[4-(2-Difluoromethyl-1-ethyl-6-oxo-6,7-dihydro-1H-purin-8-yl)-pyrazol-1-ylmethyl]-benzoic acid (319),
2-Difluoromethyl-1-ethyl-8-[1-(3-hydroxymethyl-benzyl)-1H-pyrazol-4-yl]-1,7-dihydro-purin-6-one (320),
2-Difluoromethyl-3-ethyl-6-[1-(3-methoxy-benzyl)-1H-pyrazol-4-yl]-3,5-dihydro-pyrrolo[3,2-d]pyrimidin-4-one (321),
N-[5-(2-Cyano-4-oxo-3-propyl-4,5-dihydro-3H-pyrrolo[3,2-d]pyrimidin-6-yl)-pyridin-2-yl]-3-methoxy-benzenesulfonamide (322),
2-Fluoromethyl-6-{3-[1-(3-fluoro-phenyl)-piperidin-4-yloxy]-isoxazol-5-yl}-3-propyl-3,5-dihydro-pyrrolo[3,2-d]pyrimidin-4-one (323),
2-Difluoromethyl-6-{5-[1-(3-methoxy-phenyl)-piperidin-4-yloxy]-1-methyl-1H-pyrazol-3-yl}-3-propyl-3,5-dihydro-pyrrolo[3,2-d]pyrimidin-4-one (324),
3-Ethyl-6-{6-[1-(3-methoxy-phenyl)-pyrrolidin-3-yloxy]-pyridin-3-yl}-4-oxo-4,5-dihydro-3H-pyrrolo[3,2-d]pyrimidine-2-carbonitrile (325),
2-Fluoromethyl-6-{3-[1-(3-fluoro-phenyl)-piperidin-4-yloxy]-isoxazol-5-yl}-7-hydroxy-3-propyl-3,5-dihydro-pyrrolo[3,2-d]pyrimidin-4-one (326),
2-Difluoromethyl-3-ethyl-6-[1-(3-methoxy-benzyl)-1H-pyrazol-4-yl]-7-methyl-3,5-dihydro-pyrrolo[3,2-d]pyrimidin-4-one (327),
2-Difluoromethyl-1-ethyl-8-[1-(3-methoxy-benzyl)-1H-pyrazol-4-yl]-7-methyl-1,7-dihydro-purin-6-one (328),
N-[5-(2-Cyano-7-methyl-6-oxo-1-propyl-6,7-dihydro-1H-purin-8-yl)-pyridin-2-yl]-3-methoxy-benzenesulfonamide (329),
1-(2,2-Difluoro-ethyl)-2-ethyl-8-[1-(3-methoxy-benzyl)-1H-pyrazol-4-yl]-1,7-dihydro-purin-6-one (330),
3-{3-[4-(2-Difluoromethyl-6-oxo-1-propyl-6,7-dihydro-1H-purin-8-yl)-pyrazol-1-yl]-prop-1-ynyl}-benzoic acid (331),
3-(3-{4-[1-(2,2-Difluoro-ethyl)-2-ethyl-6-oxo-6,7-dihydro-1H-purin-8-yl]-pyrazol-1-yl}-prop-1-ynyl)-benzoic acid (332),
3-{3-[4-(6-Oxo-1-propyl-2-trifluoromethyl-6,7-dihydro-1H-purin-8-yl)-pyrazol-1-yl]-prop-1-ynyl}-benzoic acid (333), and
6-Oxo-1-propyl-8-[6-(3-trifluoromethyl-benzyl)-pyridin-3-yl]-6,7-dihydro-1H-purine-2-carbonitrile (334).
